(19) United States
(12) Patent Application Publication
(10) Pub. No.: US 2025/0387339 A1
(43) Pub. Date: Dec. 25, 2025

(54) VAGINAL INSERTED ESTRADIOL PHARMACEUTICAL COMPOSITIONS AND METHODS

(71) Applicant: TherapeuticsMD, Inc., Boca Raton, FL (US)

(72) Inventors: Sebastian Mirkin, Boca Raton, FL (US); Julia M. Amadio, Boca Raton, FL (US); Brian A. Bernick, Boca Raton, FL (US)

(21) Appl. No.: 19/027,335

(22) Filed: Jan. 17, 2025

Related U.S. Application Data
(63) Continuation of application No. 15/975,733, filed on May 9, 2018, now abandoned.
Application No. 15/975,733 is a continuation of application No. 15/372,385, filed on Dec. 7, 2016, now Pat. No. 10,568,891.
Application No. 15/372,385 is a continuation-in-part of application No. 14/521,230, filed on Oct. 22, 2014, now Pat. No. 10,537,581.
Application No. 14/521,230 is a continuation-in-part of application No. PCT/US13/46443, filed on Jun. 18, 2013.
(60) Provisional application No. 61/745,313, filed on Dec. 21, 2012.
(60) Provisional application No. 61/894,411, filed on Oct. 22, 2013.
(60) Provisional application No. 61/932,140, filed on Jan. 27, 2014.
(60) Provisional application No. 62/264,309, filed on Dec. 7, 2015.
(60) Provisional application No. 62/296,552, filed on Feb. 17, 2016.
(60) Provisional application No. 62/324,838, filed on Apr. 19, 2016.
(60) Provisional application No. 62/329,940, filed on Apr. 29, 2016.
(60) Provisional application No. 62/348,820, filed on Jun. 10, 2016.

(51) Int. Cl. A61K 9/48; A61K 9/00; A61K 31/565; A61P 15/02

(52) U.S. Cl. CPC A61K 9/4825; A61K 9/0034; A61K 31/565; A61P 15/02

(57) ABSTRACT

Disclosed herein is, among other things, a soft gel vaginal pharmaceutical composition and dosage form containing solubilized estradiol for the treatment of vulvovaginal atrophy (VVA) and female sexual dysfunction (FSD).

### CROSS-REFERENCES TO RELATED APPLICATIONS

The present application is related to U.S. patent application Ser. No. 15/372,385, filed Dec. 7, 2016, which is a continuation-in-part of U.S. patent application Ser. No. 14/521,230, filed Oct. 22, 2014, and which claims priority to U.S. Provisional Pat. Appl. No. 62/264,309, filed Dec. 7, 2015; U.S. Provisional Pat. Appl. No. 62/296,552, filed Feb. 17, 2016; U.S. Provisional Pat. Appl. No. 62/324,838, filed Apr. 19, 2016; U.S. Provisional Pat. Appl. No. 62/329,940, filed Apr. 29, 2016; and U.S. Provisional Pat. Appl. No. 62/348,820, filed Jun. 10, 2016; which applications are incorporated herein by reference in their entirety.

### FIELD OF THE INVENTION

This application is directed to pharmaceutical compositions, methods, and devices related to hormone replacement therapy.

### BACKGROUND OF THE INVENTION

Postmenopausal women frequently suffer from atrophic vaginitis or vulvar and vaginal atrophy (hereinafter “vulvovaginal atrophy” or “VVA”) with symptoms including, for example, vaginal dryness, vaginal odor, vaginal or vulvar irritation or itching, dysuria (pain, burning, or stinging when urinating), dyspareunia (vaginal pain associated with sexual activity), or vaginal bleeding associated with sexual activity. Other symptoms include soreness; with urinary frequency and urgency; urinary discomfort and incontinence also occurring (“estrogen-deficient urinary state(s)”). One symptom of vaginal atrophy is an increased vaginal pH, which creates an environment more susceptible to infections. The mucosal epithelium of the VVA patients also reported to show signs of severe atrophy and upon cytological examination accompanied by an increased number of the parabasal cells and a reduced number of superficial cells.

Each of these VVA-related states manifest symptoms associated with decreased estrogenization of the vulvovaginal tissue, and can even occur in women treated with oral administration of an estrogen-based pharmaceutical drug product. Although VVA is most common with menopausal women, it can occur at any time in a woman's life cycle. VVA symptoms also interfere with sexual activity and satisfaction. Women with female sexual dysfunction (FSD) are almost 4 times more likely to have VVA than those without FSD.

Estrogen treatment has proven to be very successful in controlling menopausal symptoms, including VVA and FSD. Several studies have shown that the symptoms connected with vaginal atrophy are often relieved by estrogen treatment given either systemically or topically. The existing treatments have numerous problems, for example compliance issues with patients not completing or continuing treatment due to the problems associated with the form of treatment.

Accordingly, there remains a need in the art for treatments for VVA and FSD that overcome these limitations.

### BRIEF SUMMARY OF THE INVENTION

Disclosed herein is, among other things, a new soft gel vaginal pharmaceutical composition and dosage form containing solubilized estradiol for the treatment of VVA. The soft gel vaginal pharmaceutical composition has been designed to mitigate common limitations found with other vaginal forms of estradiol. The soft gel vaginal pharmaceutical composition eases vaginal administration, provides improved safety of insertion, minimizes vaginal discharge following administration, and provides a more effective dosage form having improved efficacy, safety and patient compliance.

According to various aspects and embodiments of this disclosure, a soft gel vaginal pharmaceutical composition as a treatment for post-menopausal women suffering with moderate to severe symptoms of VVA is provided.

In another aspect, methods of treating VVA are provided. In some embodiments, the method comprises vaginally administering to a subject in need thereof estradiol at a dosage of 4 mcg and increasing the dosage of estradiol if the subject does not exhibit an improvement in a clinical response for VVA.

In some embodiments, the estradiol is administered once daily for two weeks, then twice weekly thereafter.

In some embodiments, the estradiol is solubilized.

In some embodiments, a dosage form comprising the estradiol is manually or digitally inserted. In some embodiments, a dosage form comprising the estradiol is manually or digitally inserted about two inches into the vagina. In some embodiments, the dosage form is inserted while standing. In some embodiments, the dosage form is inserted while lying down. It will be appreciated by a person of ordinary skill in the art that an applicator can be used to deliver the estradiol into the vagina.

In some embodiments, the dosage form is a soft gelatin capsule.

In some embodiments, the subject's clinical response is measured as an improvement in one or more clinical parameters, wherein the parameters are selected from the group consisting of: (i) an increase in the percentage of vaginal superficial cells, (ii) a decrease in the percentage of vaginal parabasal cells, (iii) a decrease in vaginal pH, (iv) a decrease in the severity of moderate to severe dyspareunia, (v) a decrease in vaginal dryness, (vi) a decrease in vulvar or vaginal itching or irritation, (vii) an increase of vaginal mucosa, (viii) an assessment of standard pK parameters, (ix) a change in the Female Sexual Function Index (FSFI), (x) visual vaginal assessment, and (xi) the subject's self-reported response.

In some embodiments, the subject's clinical response is measured as an improvement in one or more clinical parameters selected from the group consisting of an increase in the percentage of vaginal superficial cells, a decrease in the percentage of vaginal parabasal cells, a decrease in vaginal pH, and a decrease in the severity of moderate to severe dyspareunia. In some embodiments, the subject's clinical response is measured as a decrease in the severity of moderate to severe dyspareunia.

In some embodiments, the subject's clinical response is measured by visual assessment or the subject's self-reported response (e.g., a subject's self-reported symptoms or self-reported change in one or more symptoms).

In some embodiments, the subject's clinical response is measured about two weeks after the first administration of the estradiol.

In some embodiments, the method comprises increasing the dosage of estradiol to 10 mcg if the subject does not exhibit an improvement in a clinical response. In some embodiments, the method comprises increasing the dosage of estradiol to an amount that is between 4-10 mcg. In some embodiments, the method comprises increasing the dosage of estradiol to 25 mcg if the subject does not exhibit an improvement in a clinical response. In some embodiments, the method comprises increasing the dosage of estradiol to an amount that is between 10-25 mcg.

In some embodiments, the method comprises increasing the dosage of estradiol from 4 mcg to an amount that is between 4-10 mcg, with 10 mcg being a preferred dosage. In some embodiments, the method comprises further increasing the dosage if the subject does not exhibit an improvement in a clinical response to the increased dosage. In some embodiments, the method further comprises increasing the dosage of estradiol to an amount that is between 10-25 mcg.

In some embodiments, the method comprises maintaining the dosage of estradiol if an improvement in a clinical response is achieved. In some embodiments, the maintained dosage of estradiol is administered twice weekly thereafter (e.g., for as long as needed to treat VVA).

In some embodiments, the method comprises decreasing the dosage of estradiol if an improvement in a clinical response is achieved. In some embodiments, the decreased dosage of estradiol is administered twice weekly thereafter (e.g., for as long as needed to treat VVA.

In another aspect, a method of treating VVA comprises vaginally administering to a subject in need thereof estradiol once daily for two weeks, then twice weekly thereafter, wherein the estradiol is administered at a dosage of 4 mcg.

In some embodiments, the estradiol is solubilized.

In some embodiments, a dosage form comprising the estradiol is manually or digitally inserted. In some embodiments, a dosage form comprising the estradiol is manually or digitally inserted about two inches into the vagina. In some embodiments, the dosage form is inserted while standing. In some embodiments, the dosage form is inserted while lying down. It will be appreciated by a person of ordinary skill in the art that an applicator can be used to deliver the estradiol into the vagina.

In some embodiments, the dosage form is a soft gelatin capsule.

In some embodiments, the method of treating VVA further comprises adjusting the dosage, e.g., to the lowest dosage of estradiol that is efficacious in treating VVA. In some embodiments, the dosage is increased to a dosage that is between 4-10 mcg, or to a dosage that is between 4-25 mcg. In some embodiments, the dosage of 4 mcg is maintained. In some embodiments, the dosage is decreased to a dosage that is less than 4 mcg, e.g., to a dosage that is between 2-4 mcg.

In another aspect, a method of treating moderate to severe dyspareunia is provided. In some embodiments, the method comprises vaginally administering to a subject in need thereof estradiol at a dosage of 4 mcg and increasing the dosage of estradiol if the subject does not exhibit an improvement in the moderate to severe dyspareunia.

In some embodiments, the estradiol is administered once daily for two weeks, then twice weekly thereafter.

In some embodiments, the estradiol is solubilized.

In some embodiments, a dosage form comprising the estradiol is manually or digitally inserted. In some embodiments, a dosage form comprising the estradiol is manually or digitally inserted about two inches into the vagina. In some embodiments, the dosage form is inserted while standing. In some embodiments, the dosage form is inserted while lying down. It will be appreciated by a person of ordinary skill in the art that an applicator can be used to deliver the estradiol into the vagina.

In some embodiments, the dosage form is a soft gelatin capsule.

In some embodiments, the subject's clinical response is measured as an improvement in one or more clinical parameters selected from the group consisting of an increase in the percentage of vaginal superficial cells, a decrease in the percentage of vaginal parabasal cells, and a decrease in vaginal pH. In some embodiments, the subject's clinical response is measured about two weeks after the first administration of the estradiol.

In some embodiments, the method comprises increasing the dosage of estradiol to 10 mcg if the subject does not exhibit an improvement in the moderate to severe dyspareunia. In some embodiments, the method comprises increasing the dosage of estradiol to 25 mcg if the subject does not exhibit an improvement in the moderate to severe dyspareunia. In some embodiments, the method comprises decreasing the dosage of estradiol if an improvement in the moderate to severe dyspareunia is achieved.

In another aspect, provided herein is a suppository comprising: a) a therapeutically effective amount of estradiol; and b) a solubilizing agent comprising a medium chain oil.

In some embodiments, the suppository includes about 1 μg to about 25 μg of estradiol. For example, the suppository can include about 1 μg to about 10 μg of estradiol; and about 10 μg to about 25 μg of estradiol.

In some embodiments, the estradiol is solubilized.

In some embodiments, the medium chain oil includes at least one C6-C12 fatty acid or a glycol, monoglyceride, diglyceride, or triglyceride ester thereof.

In some embodiments, the solubilizing agent includes at least one ester selected from the group consisting of: an ester of caproic fatty acid, an ester of caprylic fatty acid, an ester of capric fatty acid, and combinations thereof. For example, the solubilizing agent can include a caprylic/capric triglyceride.

In some embodiments, the suppository further includes a capsule. For example, the capsule can be a soft gelatin capsule.

Also provided herein is a suppository comprising: a) a therapeutically effective amount of estradiol; b) a caprylic/capric triglyceride; c) a non-ionic surfactant comprising PEG-6 palmitostearate and ethylene glycol palmitostearate; and d) a soft gelatin capsule.

In some embodiments, a suppository provided herein includes about 25 μg of estradiol, wherein administration of the suppository to a patient provides, in a plasma sample from the patient: 1) a corrected geometric mean peak plasma concentration (Cₘₐₓ) of estradiol of about 19 pg*hr/mL to about 29 pg*hr/mL; and 2) a corrected geometric mean area under the curve (AUC)₀₋₂₄ of estradiol of about 75 pg*hr/mL to about 112 pg*hr/mL.

In some embodiments, a suppository provided herein includes about 25 μg of estradiol, wherein administration of the suppository to a patient provides, in a plasma sample from the patient: 1) a corrected geometric mean peak plasma concentration (Cₘₐₓ) of estrone of about 9 pg*hr/mL to about 14 pg*hr/mL; and 2) a corrected geometric mean area under the curve (AUC)₀₋₂₄ of estrone of about 43 pg*hr/mL to about 65 pg*hr/mL.

In some embodiments, a suppository provided herein includes about 25 μg of estradiol, wherein administration of the suppository to a patient provides, in a plasma sample from the patient: 1) a corrected geometric mean peak plasma concentration (Cₘₐₓ) of estrone sulfate of about 416 pg*hr/mL to about 613 pg*hr/mL; and 2) a corrected geometric mean area under the curve (AUC)₀₋₂₄ of estrone sulfate of about 3598 pg*hr/mL to about 5291 pg*hr/mL.

In some embodiments, a suppository provided herein includes about 10 μg of estradiol, wherein administration of the suppository to a patient provides, in a plasma sample from the patient: 1) a corrected geometric mean peak plasma concentration (Cₘₐₓ) of estradiol of about 12 pg*hr/mL to about 18 pg*hr/mL; and 2) a corrected geometric mean area under the curve (AUC)₀₋₂₄ of estradiol of about 42 pg*hr/mL to about 63 pg*hr/mL. In some embodiments, the suppository further provides a corrected geometric mean time to peak plasma concentration (Tₘₐₓ) of estradiol of about 1 hrs to about 3 hrs.

In some embodiments, a suppository provided herein includes about 10 μg of estradiol, wherein administration of the suppository to a patient provides, in a plasma sample from the patient: 1) a corrected geometric mean peak plasma concentration (Cₘₐₓ) of estrone of about 4 pg*hr/mL to about 7 pg*hr/mL; and 2) a corrected geometric mean area under the curve (AUC)₀₋₂₄ of estrone of about 20 pg*hr/mL to about 31 pg*hr/mL. In some embodiments, the suppository further provides a corrected geometric mean time to peak plasma concentration (Tₘₐₓ) of estrone of about 4 hrs to about 8 hrs.

In some embodiments, a suppository provided herein includes about 10 μg of estradiol, wherein administration of the suppository to a patient provides, in a plasma sample from the patient: 1) a corrected geometric mean peak plasma concentration (Cₘₐₓ) of estrone sulfate of about 10 pg*hr/mL to about 16 pg*hr/mL; and 2) a corrected geometric mean area under the curve (AUC)₀₋₂₄ of estrone sulfate of about 56 pg*hr/mL to about 84 pg*hr/mL. In some embodiments, the suppository further provides a corrected geometric mean time to peak plasma concentration (Tₘₐₓ) of estrone sulfate of about 4 hrs to about 7 hrs.

In some embodiments, a suppository provided herein includes about 4 μg of estradiol, wherein administration of the suppository to a patient provides, in a plasma sample from the patient: 1) a corrected geometric mean peak plasma concentration (Cₘₐₓ) of estradiol of about 4 pg*hr/mL to about 8 pg*hr/mL; and 2) a corrected geometric mean area under the curve (AUC)₀₋₂₄ of estradiol of about 16 pg*hr/mL to about 26 pg*hr/mL. In some embodiments, the suppository further provides a corrected geometric mean time to peak plasma concentration (Tₘₐₓ) of estradiol of about 0.25 hrs to about 2 hrs.

In some embodiments, a suppository provided herein includes about 4 μg of estradiol, wherein administration of the suppository to a patient provides, in a plasma sample from the patient: 1) a corrected geometric mean peak plasma concentration (Cₘₐₓ) of estrone of about 1 pg*hr/mL to about 3 pg*hr/mL; and 2) a corrected geometric mean area under the curve (AUC)₀₋₂₄ of estrone of about 8 pg*hr/mL to about 13 pg*hr/mL. In some embodiments, the suppository further provides a corrected geometric mean time to peak plasma concentration (Tₘₐₓ) of estrone of about 1 hrs to about 4 hrs.

In some embodiments, a suppository provided herein includes about 4 μg of estradiol, wherein administration of the suppository to a patient provides, in a plasma sample from the patient: 1) a corrected geometric mean peak plasma concentration (Cₘₐₓ) of estrone sulfate of about 4 pg*hr/mL to about 7 pg*hr/mL; and 2) a corrected geometric mean area under the curve (AUC)₀₋₂₄ of estrone sulfate of about 22 pg*hr/mL to about 34 pg*hr/mL. In some embodiments, the suppository further provides a corrected geometric mean time to peak plasma concentration (Tₘₐₓ) of estrone sulfate of about 1 hrs to about 3 hrs.

Also provided herein is a suppository comprising about 1 μg to about 25 μg of estradiol, wherein administration of the suppository to a patient provides a corrected geometric mean peak plasma concentration (Cₘₐₓ) of estradiol that is less than about 30 pg*hr/mL. For example, administration of the suppository to a patient provides a corrected geometric mean peak plasma concentration (Cₘₐₓ) of estradiol that is less than about 18 pg*hr/mL.

In some embodiments, a suppository comprising about 1 μg to about 25 μg of estradiol is provided, wherein administration of the suppository to a patient provides a corrected geometric mean area under the curve (AUC)₀₋₂₄ of estradiol that is less than about 112 pg*hr/mL. For example, administration of the suppository to a patient provides a corrected geometric mean area under the curve (AUC)₀₋₂₄ of estradiol that is less than about 63 pg*hr/mL.

In some embodiments, a suppository comprising about 1 μg to about 25 μg of estradiol is provided, wherein administration of the suppository to a patient provides a corrected geometric mean peak plasma concentration (Cₘₐₓ) of estrone that is less than about 14 pg*hr/mL. For example, administration of the suppository to a patient provides a corrected geometric mean peak plasma concentration (Cₘₐₓ) of estrone that is less than about 7 pg*hr/mL.

In some embodiments, a suppository comprising about 1 μg to about 25 μg of estradiol is provided, wherein administration of the suppository to a patient provides a corrected geometric mean area under the curve (AUC)₀₋₂₄ of estrone that is less than about 65 pg*hr/mL. For example, administration of the suppository to a patient provides a corrected geometric mean area under the curve (AUC)₀₋₂₄ of estrone that is less than about 31 pg*hr/mL.

In some embodiments, a suppository comprising about 1 μg to about 25 μg of estradiol is provided, wherein administration of the suppository to a patient provides a corrected geometric mean peak plasma concentration (Cₘₐₓ) of estrone sulfate that is less than about 613 pg*hr/mL. For example, administration of the suppository to a patient provides a corrected geometric mean peak plasma concentration (Cₘₐₓ) of estrone sulfate that is less than about 16 pg*hr/mL.

In some embodiments, a suppository comprising about 1 μg to about 25 μg of estradiol is provided, wherein administration of the suppository to a patient provides a corrected geometric mean area under the curve (AUC)₀₋₂₄ of estrone sulfate that is less than about 5291 pg*hr/mL. For example, administration of the suppository to a patient provides a corrected geometric mean area under the curve (AUC)₀₋₂₄ of estrone sulfate that is less than about 84 pg*hr/mL.

Further provided herein is a suppository comprising about 1 μg to about 25 μg of estradiol, wherein administration of the suppository to the proximal region of the vagina of a patient provides a therapeutically effective concentration of estradiol over 24 hours in the proximal region of the vagina.

This disclosure also provides a method of treating an estrogen-deficient state, the method comprising administering to a patient in need thereof, a suppository as provided herein. In some embodiments, a method of treating vulvovaginal atrophy is provided, the method comprising administering to a patient in need thereof, a suppository as provided herein.

In some embodiments of the methods provided herein, treatment includes reducing the severity of one or more symptoms selected from the group consisting of: vaginal dryness, dyspareunia, vaginal or vulvar irritation, vaginal or vulvar burning, vaginal or vulvar itching, dysuria, and vaginal bleeding associated with sexual activity.

In some embodiments of the methods provided herein treatment includes reducing the vaginal pH of the patient. For example, treatment includes reducing the vaginal pH of the patient to a pH of less than about 5.0.

In some embodiments of the methods provided herein treatment includes a change in cell composition of the patient. For example, the change in cell composition includes reducing the number of parabasal vaginal cells or increasing the number of superficial vaginal cells. In some embodiments, the number of parabasal vaginal cells in the patient are reduced by at least about 35% (e.g., at least about 50%). In some embodiments, the number of superficial vaginal cells are increased by at least about 5% (e.g., at least about 35%).

Further provided herein is a method for reducing vaginal discharge following administration of a suppository, the method comprising administering to a patient in need thereof, a suppository provided herein, wherein the vaginal discharge following administration of the suppository is compared to the vaginal discharge following administration of a reference drug.

Also provided herein is a method for treating female sexual dysfunction in a female subject in need thereof. The method includes administering to the subject a vaginal suppository as described herein. In some embodiments, the method includes administering to the subject a vaginal suppository comprising: (a) a pharmaceutical composition comprising: a therapeutically effective amount of estradiol; a caprylic/capric triglyceride; a non-ionic surfactant comprising PEG-6 palmitostearate and ethylene glycol palmitostearate; and (b) a soft gelatin capsule; wherein the vaginal suppository includes from about 1 microgram to about 25 micrograms of estradiol; wherein estradiol is the only active hormone in the vaginal suppository. In some embodiments, the vaginal suppository does not include a hydrophilic gel-forming bioadhesive agent in the solubilizing agent. In some embodiments, treating female sexual dysfunction includes increasing the subject's desire, arousal, lubrication, satisfaction, and or/orgasms.

### BRIEF DESCRIPTION OF THE DRAWINGS

The above-mentioned features and objects of the this disclosure will become more apparent with reference to the following description taken in conjunction with the accompanying drawings wherein like reference numerals denote like elements and in which.

FIG. 1 is a flow diagram illustrating a process in accordance with various embodiments of the invention;

FIG. 2 illustrates a suppository in accordance with various embodiments of the invention;

FIG. 3 is a linear plot of mean plasma estradiol—baseline adjusted concentrations versus time (N=34);

FIG. 4 is a semi-logarithmic plot of mean plasma estradiol—baseline adjusted concentrations versus time (N=34);

FIG. 5 is a linear plot of mean plasma estrone—baseline adjusted concentrations versus time (N=33);

FIG. 6 is a semi-logarithmic plot of mean plasma estrone—baseline adjusted concentrations versus time (N=33);

FIG. 7 is a linear plot of mean plasma estrone sulfate—baseline adjusted concentrations versus time (N=24); and

FIG. 8 is a semi-logarithmic plot of mean plasma estrone sulfate—baseline adjusted concentrations versus time (N=24).

FIG. 9 is a study schematic diagram.

FIG. 10 shows the percentage change in superficial cells at 12 weeks compared to placebo.

FIG. 11 shows the percentage change in superficial cells at week 2, week 6, week 8, and week 12 compared to placebo.

FIG. 12 shows percentage change in superficial cells per dose for each of week 2, week 6, week 8, and week 12 compared to placebo.

FIG. 13 shows the percentage change in parabasal cells at 12 weeks compared to placebo.

FIG. 14 shows the percentage change in parabasal cells at week 2, week 6, week 8, and week 12 compared to placebo.

FIG. 15 shows the percentage change in parabasal cells per dose for each of week 2, week 6, week 8, and week 12 compared to placebo

FIG. 16 shows the percentage change in pH at 12 weeks compared to placebo.

FIG. 17 shows the percentage change in pH at week 2, week 6, week 8, and week 12 compared to placebo.

FIG. 18 shows the percentage change in pH per dose for each of week 2, week 6, week 8, and week 12 compared to placebo.

FIG. 19A shows the change in visual assessments from baseline to week 12 in vaginal color in a modified intent to treat (MITT) population.

FIG. 19B shows the change in visual assessments from baseline to week 12 in vaginal epithelial integrity in a modified intent to treat (MITT) population.

FIG. 19C shows the change in visual assessments from baseline to week 12 in vaginal epithelial thickness a modified intent to treat (MITT) population.

FIG. 19D shows the change in visual assessments from baseline to week 12 in vaginal secretions in a modified intent to treat (MITT) population.

FIG. 20A shows the correlation between the total sum of four visual assessments and dyspareunia at week 12 in an intent to treat (ITT) population.

FIG. 20B shows the correlation between the total sum of four visual assessments and vaginal dryness at week 12 in an intent to treat (ITT) population.

FIG. 21 shows baseline adjusted estradiol serum concentration (pg/mL) assessed on Day 1 (squares) and Week 12 (diamonds) for four treatment arms.

FIG. 22 shows baseline adjusted estradiol serum concentration (pg/mL) assessed on Day 14 (squares) and Week 12 (diamonds) for four treatment arms.

FIG. 23 shows estradiol plasma levels measured in subjects following a supine period after administration of the estradiol formulation, compared with plasma levels measured in subjects following an ambulatory period after administration of the estradiol formulation.

FIG. 24 shows mean change from baseline in Total FSFI score at Week 12.

FIG. 25A shows the mean change from baseline to week 12 in the individual FSFI lubrication score.

FIG. 25B shows the mean change from baseline to week 12 in the individual FSFI arousal score.

FIG. 25C shows the mean change from baseline to week 12 in the individual FSFI satisfaction score.

FIG. 25D shows the mean change from baseline to week 12 in the individual FSFI desire score.

FIG. 25E shows the mean change from baseline to week 12 in the individual FSFI orgasm score.

FIG. 26A shows an estradiol softgel capsule held with the larger end between the fingers.

FIG. 26B shows insertion of an estradiol softgel capsule in a reclining position. The softgel is inserted into the lower third of the vagina or about two inches into the vagina with the smaller end up.

FIG. 26C shows insertion of an estradiol softgel capsule in a standing position. The softgel is inserted into the lower third of the vagina or about two inches into the vagina with the smaller end up.

### DETAILED DESCRIPTION OF THE INVENTION

In the following detailed description of embodiments of this disclosure, reference is made to the accompanying drawings in which like references indicate similar elements, and in which is shown by way of illustration specific embodiments in which this disclosure may be practiced. These embodiments are described in sufficient detail to enable those skilled in the art to practice this disclosure, and it is to be understood that other embodiments may be utilized and that other changes may be made without departing from the scope of the this disclosure. The following detailed description is, therefore, not to be taken in a limiting sense, and the scope of this disclosure is defined only by the appended claims. As used in this disclosure, the term “or” shall be understood to be defined as a logical disjunction (i.e., and/or) and shall not indicate an exclusive disjunction unless expressly indicated as such with the terms “either,” “unless,” “alternatively,” and words of similar effect.

### I. DEFINITIONS

As used herein, the term “about,” when used to modify an amount specified in a numeric value or range, indicates that the numeric value as well as reasonable deviations from the value known to the skilled person in the art are within the intended meaning of the recited value.

The term “active pharmaceutical ingredient” (“API”) as used herein, means the active compound(s) used in formulating a drug product.

The term “co-administered” as used herein, means that two or more drug products are administered simultaneously or sequentially on the same or different days.

The term “drug product” as used herein means at least one active pharmaceutical ingredient in combination with at least one excipient and provided in unit dosage form.

The term “area under the curve” (“AUC”) refers to the area under the curve defined by changes in the blood concentration of an active pharmaceutical ingredient (e.g., estradiol or progesterone), or a metabolite of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone or estradiol), or a metabolite of the active pharmaceutical ingredient, over time.

The term “Tₘₐₓ” refers to the time that it takes for the blood concentration an active pharmaceutical ingredient (e.g., estradiol or progesterone), or a metabolite of the active pharmaceutical ingredient, to reach the maximum value.

The term “bioavailability,” which has the meaning defined in 21 C.F.R. § 320.1(a), refers to the rate and extent to which an API or active ingredient or active moiety is absorbed from a drug product and becomes available at the site of action. For example, bioavailability can be measured as the amount of API in the blood (serum or plasma) as a function of time. Pharmacokinetic (PK) parameters such as AUC, Cₘₐₓ, or Tₘₐₓ may be used to measure and assess bioavailability. For drug products that are not intended to be absorbed into the bloodstream, bioavailability may be assessed by measurements intended to reflect the rate and extent to which the API or active ingredient or active moiety becomes available at the site of action.

The term “bioequivalent,” which has the meaning defined in 21 C.F.R. § 320.1(e), refers to the absence of a significant difference in the rate and extent to which the API or active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC, Cₘₐₓ, or optionally Tₘₐₓ is within 80.00% to 125.00%.

The term “bio-identical,” “body-identical,” or “natural” used in conjunction with the hormones disclosed herein, means hormones that match the chemical structure and effect of those that occur naturally or endogenously in the human body. An exemplary natural estrogen is estradiol.

The term “bio-identical hormone” or “body-identical hormone” refers to an active pharmaceutical ingredient that is structurally identical to a hormone naturally or endogenously found in the human body (e.g., estradiol and progesterone).

The term “estradiol” refers to (17β)-estra-1,3,5(10)-triene-3,17-diol. Estradiol is also interchangeably called 17β-estradiol, oestradiol, or E2, and is found endogenously in the human body. As used herein, estradiol refers to the bio-identical or body-identical form of estradiol found in the human body having the structure:

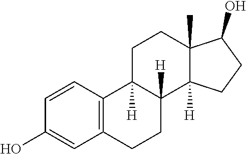

Estradiol is supplied in an anhydrous or hemi-hydrate form. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agent(s) or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as wt %).

The term “progesterone” refers to pregn-4-ene-3,20-dione. Progesterone is also interchangeably called P4 and is found endogenously in the human body. As used herein, progesterone refers to the bio-identical or body-identical form of progesterone found in the human body having the structure:

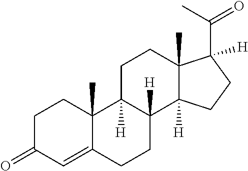

The term “solubilized progesterone” means that the progesterone or a portion thereof is solubilized or dissolved in the solubilizing agent(s) or the formulations disclosed herein. In some embodiments, the progesterone is “partially solubilized” with a portion of the progesterone being solubilized or dissolved in the solubilizing agent and a portion of the progesterone being suspended in the solubilizing agent. Partially solubilized progesterone may include progesterone that is about 1% solubilized, about 5% solubilized, about 10% solubilized, about 15% solubilized, about 20% solubilized, about 30% solubilized, about 40% solubilized, about 50% solubilized, about 60% solubilized, about 70% solubilized, about 80% solubilized, about 85% solubilized, about 90% solubilized or about 95% solubilized. In other embodiments, the progesterone is “fully solubilized” with all or substantially all of the progesterone being solubilized or dissolved in the solubilizing agent. Fully solubilized progesterone may include progesterone that is about 97% solubilized, about 98% solubilized, about 99% solubilized or about 100% solubilized. Solubility can be expressed as a mass fraction (% w/w, which is also referred to as wt %).

The terms “micronized progesterone” and “micronized estradiol,” as used herein, include micronized progesterone and micronized estradiol having an X50 particle size value below about 15 microns or having an X90 particle size value below about 25 microns. The term “X50” means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The term “glyceride” is an ester of glycerol (1,2,3-propanetriol) with acyl radicals of fatty acids and is also known as an acylglycerol. If only one position of the glycerol molecule is esterified with a fatty acid, a “monoglyceride” or “monoacylglycerol” is produced; if two positions are esterified, a “diglyceride” or “diacylglycerol” is produced; and if all three positions of the glycerol are esterified with fatty acids, a “triglyceride” or “triacylglycerol” is produced. A glyceride is “simple” if all esterified positions contain the same fatty acid; whereas a glyceride is “mixed” if the esterified positions contained different fatty acids. The carbons of the glycerol backbone are designated sn-1, sn-2 and sn-3, with sn-2 being in the middle carbon and sn-1 and sn-3 being the end carbons of the glycerol backbone.

The term “solubilizing agent” refers to an agent or combination of agents that solubilize an active pharmaceutical ingredient (e.g., estradiol or progesterone). For example and without limitation, suitable solubilizing agents include medium chain oils and other solvents and co-solvents that solubilize or dissolve an active pharmaceutical ingredient to a desirable extent. Solubilizing agents suitable for use in the formulations disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting formulation. Examples of such excipients include, but are not limited to, surfactants, emulsifiers, thickeners, colorants, flavoring agents, etc. In some embodiments, the solubilizing agent is a medium chain oil and, in some other embodiments, the medium chain oil is combined with a co-solvent(s) or other excipient(s).

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain 6 (C6) to 14 (C14) carbon atoms, 8 (C8) to 12 (C12) carbon atoms, or 8 (C8) to 10 (C10) carbon atoms.

The terms “medium chain fatty acid” and “medium chain fatty acid derivative” are used to describe fatty acids or fatty acid derivatives with aliphatic tails (i.e., carbon chains) having 6 to 14 carbon atoms. Fatty acids consist of an unbranched or branched aliphatic tail attached to a carboxylic acid functional group. Fatty acid derivatives include, for example, fatty acid esters and fatty acid containing molecules, including, without limitation, mono-, di- and triglycerides that include components derived from fatty acids. Fatty acid derivatives also include fatty acid esters of ethylene or propylene glycol. The aliphatic tails can be saturated or unsaturated (i.e., having one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. Examples of medium chain fatty acids include, without limitation, caproic acid, caprylic acid, capric acid, lauric acid, myristic acid, and derivatives thereof.

The term “oil,” as used herein, refers to any pharmaceutically acceptable oil, especially medium chain oils, and specifically excluding peanut oil, that can suspend or solubilize bioidentical progesterone or estradiol, including starting materials or precursors thereof, including micronized progesterone or micronized estradiol as described herein.

The term “medium chain oil” refers to an oil wherein the composition of the fatty acid fraction of the oil is substantially medium chain (i.e., C6 to C14) fatty acids, i.e., the composition profile of fatty acids in the oil is substantially medium chain. As used herein, “substantially” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. As used herein, “predominantly” means that greater than or equal to 50% of the fatty acid fraction of the oil is made up of medium-chain fatty acids, i.e., fatty acids with aliphatic carbon chains having 6 to 14 carbon atoms. Those of skill in the art that will readily appreciate that the terms “alkyl content” or “alkyl distribution” of an oil can be used in place of the term “fatty acid fraction” of an oil in characterizing a given oil or solubilizing agent, and these terms are used interchangeable herein. As such, medium chain oils suitable for use in the formulations disclosed herein include medium chain oils wherein the fatty acid fraction of the oil is substantially medium chain fatty acids, or medium chain oils wherein the alkyl content or alkyl distribution of the oil is substantially medium chain alkyls (C6-C12 alkyls). It will be understood by those of skill in the art that the medium chain oils suitable for use in the formulations disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3×16=48. Conversely, a triglyceride with an ECN=40 may have “mixed” fatty acid chain lengths of 8, 16 and 16; 10, 14 and 16; 8, 14 and 18; etc. Naturally occurring oils are frequently “mixed” with respect to specific fatty acids, but tend not to contain both long chain fatty acids and medium chain fatty acids in the same glycerol backbone. Thus, triglycerides with ECN's of 21-42 typically contain predominantly medium chain fatty acids; while triglycerides with ECN's of greater than 43 typically contain predominantly long chain fatty acids. For example, the ECN of corn oil triglyceride in the USP would be in the range of 51-54. Medium chain diglycerides with ECN's of 12-28 will often contain predominantly medium chain fatty chains, while diglycerides with ECN's of 32 or greater will typically contain predominantly long chain fatty acid tails. Monoglycerides will have an ECN that matches the chain length of the sole fatty acid chain. Thus, monoglyceride ECN's in the range of 6-14 contain mainly medium chain fatty acids, and monoglycerides with ECN's 16 or greater will contain mainly long chain fatty acids.

The average ECN of a medium chain triglyceride oil is typically 21-42. For example, as listed in the US Pharmacopeia (USP), medium chain triglycerides have the following composition as the exemplary oil set forth in the table below:

| Fatty-acid Tail Length | % of oil | Exemplary Oil |
|---|---|---|
| 6 | ≤2.0 | 2.0 |
| 8 | 50.0-80.0 | 70.0 |
| 10 | 20.0-50.0 | 25.0 |
| 12 | ≤3.0 | 2.0 |
| 14 | ≤1.0 | 1.0 |

and would have an average ECN of 3*[(6*0.02)+(8*0.70)+(10*0.25)+(12*0.02)+(14*0.01)]=25.8. The ECN of the exemplary medium chain triglycerides oil can also be expressed as a range (per the ranges set forth in the USP) of 24.9-27.0. For oils that have mixed mono-, di-, and triglycerides, or single and double fatty acid glycols, the ECN of the entire oil can be determined by calculating the ECN of each individual component (e.g., C8 monoglycerides, C8 diglycerides, C10 monoglycerides, and C10 monoglycerides) and taking the sum of the relative percentage of the component multiplied by the ECN normalized to a monoglyceride for each component. For example, the oil having C8 and C10 mono- and diglycerides shown in the table below has an ECN of 8.3, and is thus a medium chain oil.

| | | ECN as % of oil | ECN as % of oil |
|---|---|---|---|
| Fatty-acid | % of | (chain length) × | normalized to |
| Chain Length | oil | (% in oil) | monoglyceride |
| C8 monoglyceride | 47 | 8 × 0.47 = 3.76 | 3.76 |
| C10 monoglyceride | 8 | 10 × 0.08 = 0.8 | 0.8 |
| C8 diglyceride | 38 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN | | | 8.3 |
| (normalized to | | | |
| monoglycerides) | | | |

Expressed differently, ECN can be calculated as each chain length in the composition multiplied by its relative percentage in the oil: (8*0.85)+(10*0.15)=8.3.

The term “excipients,” as used herein, refers to non-API ingredients such as solubilizing agents, anti-oxidants, oils, lubricants, and others used in formulating pharmaceutical products.

The term “patient” or “subject” refers to an individual to whom the pharmaceutical composition is administered.

The term “pharmaceutical composition” refers to a pharmaceutical composition comprising at least a solubilizing agent and estradiol. As used herein, pharmaceutical compositions are delivered, for example via suppository (i.e., vaginal suppository), or absorbed vaginally.

The term “progestin” means any natural or man-made substance that has pharmacological properties similar to progesterone.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

The terms “atrophic vaginitis,” “vulvovaginal atrophy,” “vaginal atrophy,” and “VVA” are used herein interchangeably. The molecular morphology of VVA is well known in the medical field.

As used herein, “sexual dysfunction” refers to a condition having one or more symptoms of difficulty during any one or more stages. The dysfunction can prevent an individual from enjoying sexual activity. Non-limiting examples of symptoms of sexual dysfunction include: reduced sexual desire, reduced sexual pleasure, reduced sexual arousal and excitement, aversion to and avoidance of genital sexual contact, inability to attain or maintain arousal, and persistent or recurrent delay of, or absence of orgasm. Sexual dysfunction may be lifelong (no effective performance ever) or acquired (after a period of normal function); generalized or limited to certain situations or certain partners; and total or partial.

As used herein, “sexual desire” refers to the frequency of wanting to engage in sexual activity and/or the frequency of engaging in sexual activity as perceived by the individual. Sexual desire can be expressed, for example, in one or more cognitive activities, including the frequency of sexual thoughts, the extent of enjoyment of movies, books, music, etc. having sexual content and/or the extent of enjoyment or pleasure of thinking and fantasizing about sex as perceived by the individual.

As used herein, “sexual arousal” refers to the frequency of becoming sexually aroused, how readily sexual arousal occurs and/or if arousal is maintained, as perceived by the individual. Psychologically, arousal can include factors such as increased desire for sexual activity and excitement related to sexual activity. Physiologically, arousal can include increased blood flow to the genitals, causing clitoral engorgement, as well as vaginal lubrication.

As used herein, “lubrication” refers to wetness in and around the vagina before, during, or after sexual activity. Increasing lubrication can include increasing the frequency of lubrication; decreasing the difficulty of becoming lubricated; and/or decreasing the difficulty in maintaining lubrication.

As used herein, “satisfaction” refers to one or more positive emotions (e.g., contentment, fulfillment, gratification, and the like) related to a sexual activity or sexual relationship. Satisfaction can include, for example, satisfaction with occurrence of sexual arousal or orgasm, satisfaction with the amount of closeness with a partner, and satisfaction with overall sex life.

As used herein, “orgasm” refers to the highest point of sexual excitement characterized by a subjective experience of intense pleasure marked normally by vaginal contractions in females. Increasing orgasm can include increasing the frequency, duration, and/or intensity of orgasms in a subject. Increasing orgasm can also include decreasing the difficulty of reaching orgasm.

### II. INTRODUCTION

Provided herein are pharmaceutical compositions comprising solubilized estradiol designed to be absorbed vaginally. The pharmaceutical compositions disclosed herein are designed to be absorbed and have their therapeutic effect locally, e.g., in vaginal or surrounding tissue. Further disclosed herein are data demonstrating efficacy of the pharmaceutical compositions disclosed, as well as methods relating to the pharmaceutical compositions. Generally, the pharmaceutical compositions disclosed herein are useful in VVA, dyspareunia, and other indications caused by decrease or lack of estrogen. In some embodiments, the pharmaceutical compositions disclosed herein are useful for improving one or more clinical parameters, such as increasing the percentage of vaginal superficial cells, decreasing the percentage of vaginal parabasal cells, decreasing vaginal pH, decreasing the severity of moderate to severe dyspareunia, decreasing vaginal dryness, decreasing vulvar or vaginal itching or irritation, or increasing vaginal mucosa.

Additional aspects and embodiments of this disclosure include: providing increased patient ease of use while potentially minimizing certain side effects from inappropriate insertion, minimizing incidence of vulvovaginal mycotic infection compared to incidence of vulvovaginal mycotic infection due to usage of other vaginally applied estradiol products; and, improved side effect profile (e.g., pruritus) compared to, for example, VAGIFEM® (estradiol vaginal tablets, Novo Nordisk; Princeton, NJ).

### III. PHARMACEUTICAL COMPOSITIONS

### Functionality

According to embodiments, the pharmaceutical compositions disclosed herein are alcohol-free or substantially alcohol-free. The pharmaceutical compositions offer provide for improved patient compliance because of improvements over the prior offering. According to embodiments, the pharmaceutical compositions disclosed herein are encapsulated in soft gelatin capsules, which improve comfort during use. According to embodiments, the terms “soft gelatin capsule,” “capsule,” “softgel,” “insert”, and “suppository” are used interchangeably. According to embodiments, the pharmaceutical compositions are substantially liquid, which are more readily absorbed in the vaginal tissue, and also are dispersed over a larger surface area of the vaginal tissue.

### Estradiol

According to embodiments, the pharmaceutical compositions disclosed herein are for vaginal insertion in a single or multiple unit dosage form. According to embodiments, the estradiol in the pharmaceutical compositions is at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% solubilized. According to embodiments and where the estradiol is not 100% solubilized, the remaining estradiol is present in a micronized (crystalline) form that is absorbable by the body and retains biological functionality, either in its micronized form or in another form which the micronized form is converted to after administration.

According to embodiments, all or some of the estradiol is solubilized in a solubilizing agent during manufacturing process. According to embodiments, all or some of the estradiol is solubilized following administration (e.g., the micronized portion where the estradiol is not 100% solubilized is solubilized in a body fluid after administration). According to embodiments, because the estradiol is solubilized, the solubilizing agents taught herein, with or without additional excipients other than the solubilizing agents, are liquid or semi-solid. To the extent the estradiol is not fully solubilized at the time of administration/insertion, the estradiol should be substantially solubilized at a body temperature (average of 37° C.) and, generally, at the pH of the vagina (ranges from 3.8 to 4.5 in healthy patients; or 4.6 to 6.5 in VVA patients).

According to embodiments, the estradiol can be added to the pharmaceutical compositions disclosed herein as estradiol, estradiol hemihydrate, or other grade estradiol forms used in pharmaceutical compositions or formulations.

According to embodiments, estradiol dosage strengths vary. Estradiol (or estradiol hemihydrate, for example, to the extent the water content of the estradiol hemihydrate is accounted for) dosage strength of is from at least about 1 microgram (μg or μg) to at least about 50 μg. Specific dosage embodiments contain at least about: 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, or 50 μg estradiol. According to embodiments, the pharmaceutical compositions contain at least about 2.5 μg, 4 μg, 6.25 μg, 7.5 μg, 12.5 μg, or 18.75 μg of estradiol. According to embodiments, the pharmaceutical compositions contain from about 1 μg to about 10 μg, from about 3 μg to about 7 μg, from about 4 μg to about 25 μg, from about 7.5 μg to 12.5 μg, from about 10 μg to about 25 μg, about 1 μg, about 2.5 μg, from about 23.5 μg to 27.5 μg, from about 7.5 μg to 22.5 μg, or from 10 μg to 25 μg of estradiol. The lowest clinically effective dose of estradiol is used for treatment of VVA and other indications set forth herein. In some embodiments, the estradiol dosage is about 4 μg. In one embodiment, the estradiol dosage is about 10 μg. In another embodiment, the estradiol dosage is about 25 μg.

### Solvent System

According to embodiments, the solvent system that solubilizes the estradiol are medium chain fatty acid based solvents, together with other excipients. According to embodiments, the solvent system includes non-toxic, pharmaceutically acceptable solvents, co-solvents, surfactants, and other excipients suitable for vaginal delivery or absorption.

According to embodiments, oils having medium chain fatty acids as a majority component are used as solubilizing agents to solubilize estradiol. According to embodiments, the solubilizing agents comprise medium chain fatty acid esters (e.g., esters of glycerol, ethylene glycol, or propylene glycol) or mixtures thereof. According to embodiments, the medium chain fatty acids comprise chain lengths from C6 to C14. According to embodiments the medium chain fatty acids comprise chain lengths from C6 to C12. According to embodiments the medium chain fatty acids substantially comprise chain lengths from C8-C10. ECN's for medium chain oils will be in the range of 21-42 for triglycerides, 12-28 for diglycerides, and 6-14 for monoglycerides.

According to embodiments, the medium chain fatty acids are saturated. According to embodiments, the medium chain fatty acids are predominantly saturated, i.e., greater than about 60% or greater than about 75% saturated.

According to embodiments, estradiol is soluble in the solubilizing agent at room temperature, although it may be desirable to warm certain solubilizing agents during manufacture to improve viscosity. According to embodiments, the solubilizing agent is liquid at between room temperature and about 50° C., at or below 50° C., at or below 40° C., or at or below 30° C.

According to embodiments, the solubility of estradiol in the medium chain oil, medium chain fatty acid, or solubilizing agent (or oil/surfactant) is at least about 0.01 wt %, 0.02 wt %, 0.05 wt %, 0.06 wt %, 0.08 wt %, 0.1 wt %, 0.2 wt %, 0.3 wt %, 0.4 wt %, 0.5 wt %, 0.6 wt %, 0.7 wt %, 0.8 wt %, 0.9 wt %, 1.0 wt %, or higher.

According to embodiments, medium chain solubilizing agents include, for example and without limitation saturated medium chain fatty acids: caproic acid (C6), enanthic acid (C7), caprylic acid (C8), pelargonic acid (C9), capric acid (C10), undecylic acid (C11), lauric acid (C12), tridecylic acid (C13), or myristic acid (C14). According to embodiments, the solubilizing agent includes oils made of these free medium chain fatty acids, oils of medium chain fatty acid esters of glycerin, propylene glycol, or ethylene glycol, or combinations thereof. These examples comprise predominantly saturated medium chain fatty acids (i.e., greater than 50% of the fatty acids are medium chain saturated fatty acids). According to embodiments, predominantly C6 to C12 saturated fatty acids are contemplated. According to embodiments, the solubilizing agent is selected from at least one of a solvent or co-solvent.

According to embodiments, glycerin based solubilizing agents include: mono-, di-, or triglycerides and combinations and derivatives thereof. Exemplary glycerin based solubilizing agents include MIGLYOLs®, which are caprylic/capric triglycerides (SASOL Germany GMBH, Hamburg). MIGLYOLs includes MIGLYOL 810 (caprylic/capric triglyceride), MIGLYOL 812 (caprylic/capric triglyceride), MIGLYOL 816 (caprylic/capric triglyceride), and MIGLYOL 829 (caprylic/capric/succinic triglyceride). Other caprylic/capric triglyceride solubilizing agents are likewise contemplated, including, for example: caproic/caprylic/capric/lauric triglycerides; caprylic/capric/linoleic triglycerides; caprylic/capric/succinic triglycerides. According to embodiments, CAPMUL MCM, medium chain mono- and di-glycerides, is the solubilizing agent. Other and triglycerides of fractionated vegetable fatty acids, and combinations or derivatives thereof can be the solubilizing agent, according to embodiments. For example, the solubilizing agent can be 1,2,3-propanetriol (glycerol, glycerin, glycerine) esters of saturated coconut and palm kernel oil and derivatives thereof.

Ethylene and propylene glycols (which include polyethylene and polypropylene glycols) solubilizing agents include: glyceryl mono- and di-caprylates; propylene glycol monocaprylate (e.g., CAPMUL® PG-8 (the CAPMUL brands are owned by ABITEC, Columbus, Ohio)); propylene glycol monocaprate (e.g., CAPMUL PG-10); propylene glycol mono- and dicaprylates; propylene glycol mono- and dicaprate; diethylene glycol mono ester (e.g., TRANSCUTOL®, 2-(2-ethoxyethoxy)ethanol, GATTEFOSSÉ SAS); and diethylene glycol monoethyl ether. Other combinations of mono- and di-esters of propylene glycol or ethylene glycol are expressly contemplated are the solubilizing agent.

According to embodiments, the solubilizing agent includes combinations of mono- and di-propylene and ethylene glycols and mono-, di-, and triglyceride combinations. According to embodiments, polyethylene glycol glyceride (GELUCIRE®, GATTEFOSSÉ SAS, Saint-Priest, France) can be used herein as the solubilizing agent or as a surfactant. For example, GELUCIRE 44/14 (PEG-32 glyceryl laurate EP), a medium chain fatty acid esters of polyethylene glycol, is a polyethylene glycol glyceride composed of mono-, di- and triglycerides and mono- and diesters of polyethylene glycol.

According to embodiments, commercially available fatty acid glycerol and glycol ester solubilizing agents are often prepared from natural oils and therefore may comprise components in addition to the fatty acid esters that predominantly comprise and characterize the solubilizing agent. Such other components may be, e.g., other fatty acid mono-, di-, and triglycerides; fatty acid mono- and diester ethylene or propylene glycols, free glycerols or glycols, or free fatty acids, for example. In some embodiments, when an oil/solubilizing agent is described herein as a saturated C₈ fatty acid mono- or diester of glycerol, the predominant component of the oil, i.e., >50 wt % (e.g., >75 wt %, >85 wt % or >90 wt %) is caprylic monoglycerides and caprylic diglycerides. For example, the Technical Data Sheet by ABITEC for CAPMUL MCM C8 describes CAPMUL MCM C8 as being composed of mono and diglycerides of medium chain fatty acids (mainly caprylic) and describes the alkyl content as ≤1% C6, ≥95% C8, ≤5% C10, and ≤1.5% C12 and higher.

For example, MIGLYOL 812 is a solubilizing agent that is generally described as a C8-C10 triglyceride because the fatty acid composition is at least about 80% triglyceride esters of caprylic acid (C8) and capric acid (C10). However, it also includes small amounts of other fatty acids, e.g., less than about 5% of caproic acid (C6), lauric acid (C12), and myristic acid (C14). The product information sheet for various MIGLYOLs illustrate the various fatty acid components as follows:

| Tests | 810 | 812 | 818 | 829 | 840 |
|---|---|---|---|---|---|
| Caproic acid | max. 2.0 | max. 2.0 | max. 2 | max. 2 | max. 2 |
| (C6:0) | | | | | |
| Capiylic acid | 65.0-80.0 | 50.0-65.0 | 45-65 | 45-55 | 65-80 |
| (C8:0) | | | | | |
| Capric acid | 20.0-35.0 | 30.0-45.0 | 30-45 | 30-40 | 20-35 |
| (C10:0) | | | | | |
| Lauric acid | max. 2 | max. 2 | max. 3 | max. 3 | max. 2 |
| (C12:0) | | | | | |
| Myristic acid | max. 1.0 | max. 1.0 | max. 1 | max. 1 | max. 1 |
| (C14:0) | | | | | |
| Linoleic acid | — | — | 2-5 | — | — |
| (C18:2) | | | | | |
| Succinic acid | — | — | — | 15-20 | — |
| ECN | 25.5-26.4 | 26.1-27 | 26.52-28.56 | 26-27.6 | 25.5-26.4 |

According to embodiments, anionic or non-ionic surfactants may be used in pharmaceutical compositions containing solubilized estradiol. Ratios of solubilizing agent(s) to surfactant(s) vary depending upon the respective solubilizing agent(s) and the respective surfactant(s) and the desired physical characteristics of the resultant pharmaceutical composition. For example and without limitation, CAPMUL MCM and a non-ionic surfactant may be used at ratios including 65:35, 70:30, 75:25, 80:20, 85:15 and 90:10. Other non-limiting examples include: CAPMUL MCM and GELUCIRE 39/01 used in ratios including, for example and without limitation, 6:4, 7:3, and 8:2; CAPMUL MCM and GELUCIRE 43/01 used in ratios including, for example and without limitation, 7:3, and 8:2; CAPMUL MCM and GELUCIRE 50/13 used in ratios including, for example and without limitation, 7:3, and 8:2, and 9:1.

### Other Excipients

According to embodiments, the pharmaceutical composition further includes a surfactant. The surfactant can be a nonionic surfactant, cationic surfactant, anionic surfactant, or mixtures thereof. Suitable surfactants include, for example, water-insoluble surfactants having a hydrophilic-lipophilic balance (HLB) value less than 12 and water-soluble surfactants having a HLB value greater than 12. Surfactants that have a high HLB and hydrophilicity, aid the formation of oil-water droplets. The surfactants are amphiphilic in nature and are capable of dissolving or solubilizing relatively high amounts of hydrophobic drug compounds.

Non-limiting examples, include, Tween, Dimethylacetamide (DMA), Dimethyl sulfoxide (DMSO), Ethanol, Glycerin, N-methyl-2-pyrrolidone (NMP), PEG 300, PEG 400, Poloxamer 407, Propylene glycol, Phospholipids, Hydrogenated soy phosphatidylcholine (HSPC), Distearoylphosphatidylglycerol (DSPG), L-α-dimyristoylphosphatidylcholine (DMPC), L-α-dimyristoylphosphatidylglycerol (DMPG), Polyoxyl 35 castor oil (CREMOPHOR EL, CREMOPHOR ELP), Polyoxyl 40 hydrogenated castor oil (Cremophor RH 40), Polyoxyl 60 hydrogenated castor oil (CREMOPHOR RH 60), Polysorbate 20 (TWEEN 20), Polysorbate 80 (TWEEN 80), d-α-tocopheryl polyethylene glycol 1000 succinate (TPGS), Solutol HS-15, Sorbitan monooleate (SPAN 20), PEG 300 caprylic/capric glycerides (SOFTIGEN 767), PEG 400 caprylic/capric glycerides (LABRASOL), PEG 300 oleic glycerides (LABRAFIL M-1944CS), Polyoxyl 35 Castor oil (ETOCAS 35), Glyceryl Caprylate (Mono- and Diglycerides) (IMWITOR), PEG 300 linoleic glycerides (LABRAFIL M-2125CS), Polyoxyl 8 stearate (PEG 400 monosterate), Polyoxyl 40 stearate (PEG 1750 monosterate), and combinations thereof. Additionally, suitable surfactants include, for example, polyoxyethylene derivative of sorbitan monolaurate such as polysorbate, caprylocaproyl macrogol glycerides, polyglycolyzed glycerides, and the like.

According to embodiments, the non-ionic surfactant is selected from one or more of glycerol and polyethylene glycol esters of long chain fatty acids, for example, lauroyl macrogol-32 glycerides or lauroyl polyoxyl-32 glycerides, commercially available as GELUCIRE, including, for example, GELUCIRE 39/01 (glycerol esters of saturated C12-C18 fatty acids), GELUCIRE 43/01 (hard fat NF/JPE) and GELUCIRE 50/13 (stearoyl macrogol-32 glycerides EP, stearoyl polyoxyl-32 glycerides NF, stearoyl polyoxylglycerides (USA FDA IIG)). These surfactants may be used at concentrations greater than about 0.01%, and typically in various amounts of about 0.01%-10.0%, 10.1%-20%, and 20.1%-30%. In some embodiments, surfactants may be used at concentrations of about 1% to about 10% (e.g., about 1% to about 5%, about 2% to about 4%, about 3% to about 8%).

According to embodiments, non-ionic surfactants include, for example and without limitation: one or more of oleic acid, linoleic acid, palmitic acid, and stearic acid. According to embodiments, non-ionic surfactants comprise polyethylene sorbitol esters, including polysorbate 80, which is commercially available under the trademark TWEEN® 80 (polysorbate 80) (Sigma Aldrich, St. Louis, MO). Polysorbate 80 includes approximately 60%-70% oleic acid with the remainder comprising primarily linoleic acids, palmitic acids, and stearic acids. Polysorbate 80 may be used in amounts ranging from about 5 to 50%, and according to embodiments, about 30% of the pharmaceutical composition total mass.

According to embodiments, the non-ionic surfactant includes PEG-6 palmitostearate and ethylene glycol palmitostearate, which are available commercially as TEFOSE® 63 (GATTEFOSSÉ SAS, Saint-Priest, France), which can be used with, for example, CAPMUL MCM having ratios of MCM to TEFOSE 63 of, for example, 8:2 or 9:1. According to embodiments, other solubilizing agents/non-ionic surfactants combinations include, for example, MIGLYOL 812:GELUCIRE 50/13 or MIGLYOL 812:TEFOSE 63.

According to embodiments, the surfactant can be an anionic surfactant, for example: ammonium lauryl sulfate, dioctyl sodium sulfosuccinate, perfluoro-octane sulfonic acid, potassium lauryl sulfate, or sodium stearate. Cationic surfactants are also contemplated.

According to embodiments, non-ionic or anionic surfactants can be used alone with at least one solubilizing agent or can be used in combination with other surfactants. Accordingly, such surfactants, or any other excipient as set forth herein, may be used to solubilize estradiol. The combination of solubilizing agent, surfactant, and other excipients should be designed whereby the estradiol is absorbed into the vaginal tissue. According to embodiments, the pharmaceutical composition will result in minimal vaginal discharge.

According to embodiments, the pharmaceutical composition further includes at least one thickening agent. Generally, a thickening agent is added when the viscosity of the pharmaceutical composition results less than desirable absorption. According to embodiments, the surfactant(s) disclosed herein may also provide thickening of the pharmaceutical composition that, upon release, will aid the estradiol in being absorbed by the vaginal mucosa while minimizing vaginal discharge. Examples of thickening agents include: hard fats; propylene glycol; a mixture of hard fat EP/NF/JPE, glyceryl ricinoleate, ethoxylated fatty alcohols (ceteth-20, steareth-20) EP/NF (available as OVUCIRE® 3460, GATTEFOSSÉ, Saint-Priest, France); a mixture of hard fat EP/NF/JPE, glycerol monooleate (type 40) EP/NF (OVUCIRE WL 3264; a mixture of hard fat EP/NF/JPE, glyceryl monooleate (type 40) EP/NF (OVUCIRE WL 2944); a non-ionic surfactant comprising PEG-6 stearate, ethylene glycol palmitostearate, and PEG-32 stearate; TEFOSE 63 or a similar product; and a mixture of various hard fats (WITEPSOL®, Sasol Germany GmbH, Hamburg, Germany). Other thickening agents such as the alginates, certain gums such as xanthan gums, agar-agar, iota carrageenans, kappa carrageenans, etc. Several other compounds can act as thickening agents like gelatin, and polymers like HPMC, PVC, and CMC. According to embodiments, the viscosity of pharmaceutical compositions in accordance with various embodiments may comprise from about 50 cps to about 1000 cps at 25° C. A person of ordinary skill in the art will readily understand and select from suitable thickening agents.

According to embodiments, the thickening agent is a non-ionic surfactant. For example, polyethylene glycol saturated or unsaturated fatty acid ester or diester is the non-ionic surfactant thickening agent. In embodiments, the non-ionic surfactant includes a polyethylene glycol long chain (C16-C20) fatty acid ester and further includes an ethylene glycol long chain fatty acid ester, such as PEG-fatty acid esters or diesters of saturated or unsaturated C16-C18 fatty acids, e.g., oleic, lauric, palmitic, and stearic acids. In embodiments, the non-ionic surfactant includes a polyethylene glycol long chain saturated fatty acid ester and further includes an ethylene glycol long chain saturated fatty acid ester, such as PEG- and ethylene glycol-fatty acid esters of saturated C16-C18 fatty acids, e.g., palmitic and stearic acids. Such non-ionic surfactant can comprise PEG-6 stearate, ethylene glycol palmitostearate, and PEG-32 stearate, such as but not limited to TEFOSE 63.

According to embodiments, TEFOSE 63 is used to provide additional viscosity and/or spreadability in the vagina so as to retard flow of the composition out of the vagina. While the pharmaceutical composition remains liquid, the viscosity of such a pharmaceutical composition causes the liquid to remain in the API absorption area whereby the pharmaceutical composition is substantially absorbed by the tissue. Surprisingly, the addition of an excipient to increase the viscosity and/or spreadability of the pharmaceutical compositions herein allows the administration of a pharmaceutical composition that is liquid at body temperature but does not excessively discharge from the vagina when the patient is standing, which allows the patients to be ambulatory after administration of the pharmaceutical compositions.

According to embodiments, the non-ionic surfactant used as a thickening agent is not hydrophilic and has good emulsion properties. An illustrative example of such surfactant is TEFOSE 63, which has a hydrophilic-lipophilic balance (HLB) value of about 9-10.

According to embodiments, the pharmaceutical composition further includes one or more mucoadherent agents to improve vaginal absorption of the estradiol by, for example, increasing the viscosity of of the pharmaceutical composition whereby flow out of the vagina is retarded. According to other embodiments, alone or in addition to changes in viscosity, the mucoadhesive agent causes the pharmaceutical composition to adhere to the vaginal tissue chemically or mechanically. For example, a mucoadherent agent can be present to aid the pharmaceutical composition with adherence to the mucosa upon activation with water. According to embodiments, polycarbophil is the mucoadherent agent. According to embodiments, other mucoadherent agents include, for example and without limitation: poly (ethylene oxide) polymers having a molecular weight of from about 100,000 to about 900,000; chitosans; carbopols including polymers of acrylic acid crosslinked with allyl sucrose or allyl pentaerythritol; polymers of acrylic acid and C10-C30 alkyl acrylate crosslinked with allyl pentaerythritol; carbomer homopolymer or copolymer that contains a block copolymer of polyethylene glycol and a long chain alkyl acid ester; and the like. According to embodiments, various hydrophilic polymers and hydrogels may be used as the mucoadherent agent. According to certain embodiments, the polymers or hydrogels can swell in response to contact with vaginal tissue or secretions, enhancing moisturizing and mucoadherent effects. The selection and amount of hydrophilic polymer may be based on the selection and amount of solubilizing agent. In some embodiments, the pharmaceutical composition includes a hydrophilic polymer but optionally excludes a gelling agent. In embodiments having a hydrogel, from about 5% to about 10% of the total mass may comprise the hydrophilic polymer. In further embodiments, hydrogels may be employed. A hydrogel may comprise chitosan, which swell in response to contact with water. In various embodiments, a cream pharmaceutical composition may comprise PEG-90M. In some embodiments, a mucoadherent agent is present in the pharmaceutical formulation, in the soft gel capsule, or both.

According to embodiments, the pharmaceutical compositions include one or more thermoreversible gels, typically of the hydrophilic nature including for example and without limitation, hydrophilic sucrose and other saccharide-based monomers (U.S. Pat. No. 6,018,033, which is incorporated by reference).

According to embodiments, the pharmaceutical composition further includes a lubricant. In some embodiments, a lubricant can be present to aid in formulation of a dosage form. For example, a lubricant may be added to ensure that capsules or tablets do not stick to one another during processing or upon storage. Any suitable lubricant may be used. For example, lecithin, which is a mixture of phospholipids, is the lubricant.

According to embodiments, the pharmaceutical composition further includes an antioxidant. Any suitable anti-oxidant may be used. For example, butylated hydroxytoluene, butylated hydroxyanisole, and Vitamin E TPGS.

According to embodiments, the pharmaceutical composition includes about 20% to about 80% solubilizing agent by weight, about 0.1% to about 5% lubricant by weight, and about 0.01% to about 0.1% antioxidant by weight.

The choice of excipient will depend on factors such as, for example, the effect of the excipient on solubility and stability. Additional excipients used in various embodiments may include colorants and preservatives. Examples of colorants include FD&C colors (e.g., blue No. 1 and Red No. 40), D&C colors (e.g., Yellow No. 10), and opacifiers (e.g., Titanium dioxide). According to embodiments, colorants, comprise about 0.1% to about 2% of the pharmaceutical composition by weight. According to embodiments, preservatives in the pharmaceutical composition comprise methyl and propyl paraben, in a ratio of about 10:1, and at a proportion of about 0.005% and 0.05% by weight.

Generally, the solubilizing agents, excipients, other additives used in the pharmaceutical compositions described herein, are non-toxic, pharmaceutically acceptable, compatible with each other, and maintain stability of the pharmaceutical composition and the various components with respect to each other. Additionally, the combination of various components that comprise the pharmaceutical compositions will maintain will result in the desired therapeutic effect when administered to a subject.

### Solubility of Estradiol

According to embodiments, solubilizing agents comprising mixtures of medium chain fatty acid glycerides, e.g., C₆-C12, C₈-C12, or C₈-C10 fatty acid mono- and diglycerides or mono-, di-, and triglycerides dissolve estradiol. As illustrated in the Examples, good results were obtained with solubilizing agents that are predominantly a mixture of C8-C10 saturated fatty acid mono- and diglycerides, or medium chain triglycerides (e.g., MIGLYOL 810 or 812). Longer chain glycerides appear to be not as well suited for dissolution of estradiol.

A solubilizing agent comprising propylene glycol monocaprylate (e.g., CAPRYOL) and 2-(2-Ethoxyethoxy)ethanol (e.g., TRANSCUTOL) solubilized estradiol well.

### IV. MANUFACTURE OF THE PHARMACEUTICAL COMPOSITION

According to embodiments, the pharmaceutical composition is prepared via blending estradiol with a pharmaceutically acceptable solubilizing agent, including for example and without limitation, at least one medium chain fatty acid such as medium chain fatty acids consisting of at least one mono-, di-, or triglyceride, or derivatives thereof, or combinations thereof. According to embodiments, the pharmaceutical composition also includes at least one glycol or derivatives thereof or combinations thereof or combinations of at least one glyceride and glycol. The glycol(s) may be used as solubilizing agents or to adjust viscosity and, thus, may be considered thickening agents, as discussed further herein. Optionally added are other excipients including, for example and without limitation, anti-oxidants, lubricants, and the like. According to embodiments, the pharmaceutical composition includes sufficient solubilizing agent to fully solubilize the estradiol. It is expressly understood, however, the other volumes of solubilizing agent can be used depending on the level of estradiol solubilization desired. Persons of ordinary skill in the art will know and understand how to determine the volume of solubilizing agent and other excipients depending on the desired percent of estradiol to be solubilized in the pharmaceutical composition.

In illustrative embodiments, GELUCIRE 44/14 (lauroyl macrogol-32 glycerides EP, lauroyl polyoxyl-32 glycerides NF, lauroyl polyoxylglycerides (USA FDA IIG)) is heated to about 65° C. and CAPMUL MCM is heated to about 40° C. to facilitate mixing of the oil and non-ionic surfactant, although such heating is not necessary to dissolve the estradiol.

Specific Examples disclosed herein provide additional principles and embodiments illustrating the manufactures of the pharmaceutical compositions disclosed herein.

### V. DELIVERY VEHICLE

Generally, the pharmaceutical compositions described herein delivered intravaginally inside of a delivery vehicle, for example a capsule. According to embodiments, the capsules are soft capsules made of materials well known in the pharmaceutical arts, for example, gelatin. However, according to embodiments, the delivery vehicle is integral with the pharmaceutical composition (i.e., the pharmaceutical composition is the delivery vehicle). In such embodiments the pharmaceutical compositions is a gel, cream, ointment, tablet, or other preparation that is directly applied and absorbed vaginally.

According to embodiments, the capsules do not contain one or more of the following: a hydrophilic gel-forming bioadhesive agent, a lipophilic agent, a gelling agent for the lipophilic agent, and/or a hydrodispersible agent. According to embodiments, the capsules do not contain a hydrophilic gel-forming bioadhesive agent selected from: carboxyvinylic acid, hydroxypropylcellulose, carboxymethylcellulose, gelatin, xanthan gum, guar gum, aluminum silicate, and mixtures thereof. According to embodiments, the capsules do not contain a lipophilic agent selected from: a liquid triglyceride, a solid triglyceride (with a melting point of about 35° C.), carnauba wax, cocoa butter, and mixtures thereof. According to embodiments, the capsules do not contain a hydrophobic colloidal silica gelling agent. According to embodiments, the capsules do not contain a hydrodispersible agent selected from: polyoxyethylene glycol, polyoxyethylene glycol 7-glyceryl-cocoate, and mixtures thereof. In some embodiments, the estradiol is formulated as a liquid composition consisting of a therapeutically effective amount of estradiol; a caprylic/capric triglyceride; and a non-ionic surfactant comprising PEG-6 palmitostearate and ethylene glycol palmitostearate. In such embodiments, a hydrophilic gel-forming bioadhesive agent in the liquid composition. In some such embodiments, the liquid composition is contained with a gelatin capsule as described herein. In some such embodiments, the capsule comprises gelatin and optionally one or more further components selected from the group consisting of gelatin, hydrolyzed gelatin, sorbitol-sorbitan solution, water, glycerin, titanium dioxide, FD&C Red #40, ethanol, ethyl acetate, propylene glycol, polyvinyl acetate phthalate, isopropyl alcohol, polyethylene glycol, and ammonium hydroxide.

According to embodiments, the delivery vehicle is designed for ease of insertion. According to embodiments, the delivery vehicle is sized whereby it can be comfortably inserted into the vagina. According to embodiments, the delivery vehicle is prepared in a variety of geometries. For example, the delivery vehicle is shaped as a tear drop, a cone with frustoconical end, a cylinder, a cylinder with larger “cap” portion, or other shapes suitable for and that ease insertion into the vagina. According to embodiments, the delivery vehicle is used in connection with an applicator. According to other embodiments, the delivery vehicle is inserted digitally.

According to embodiments, a method for the treatment of VVA, including dyspareunia, vaginal dryness, and estrogen-deficient urinary states (including urinary tract infections), is provided wherein a composition for the treatment of VVA is digitally inserted approximately two inches into the vagina or in the third of the vagina closest to the opening of the vagina and results in at least one of an improvement in a clinical response for VVA (e.g., a decrease in the severity of moderate to severe dyspareunia); improved compliance compared to other products for the treatment of VVA; improved user experience compared to other products for the treatment of VVA; and statistically significantly improved symptoms of VVA, compared to placebo or baseline within one of two, four, six, eight, ten, or twelve or more weeks after initiation of administration. According to embodiments, a method for the treatment of VVA, including dyspareunia, vaginal dryness, and estrogen-deficient urinary states (including urinary tract infections), is provided wherein a delivery vehicle containing a composition for the treatment of VVA and a tear drop shape as disclosed herein is inserted approximately two inches into the vagina or in the third of the vagina closest to the opening of the vagina and results in at least one of an improvement in a clinical response for VVA (e.g., a decrease in the severity of moderate to severe dyspareunia); improved compliance compared to other products for the treatment of VVA; improved user experience compared to other products for the treatment of VVA; and statistically significantly improved symptoms of VVA, compared to placebo or baseline within one of two, four, six, eight, ten, or twelve or more weeks after initiation of administration.

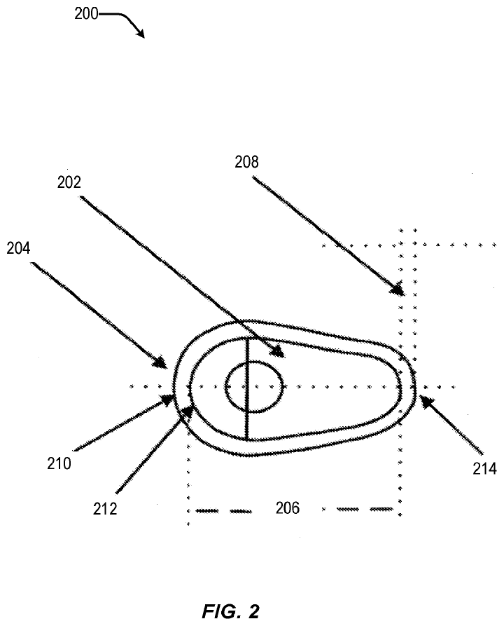

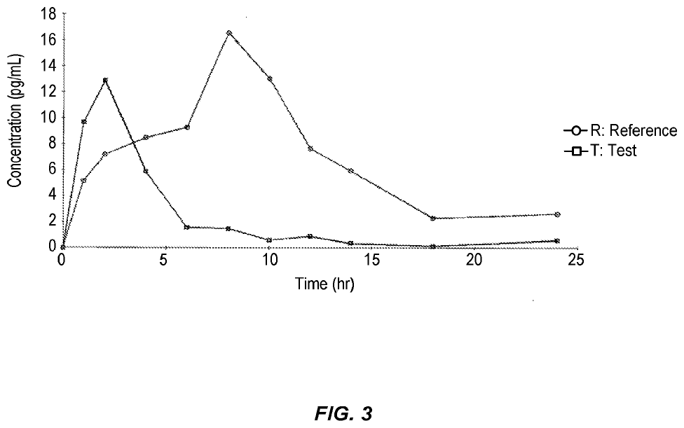

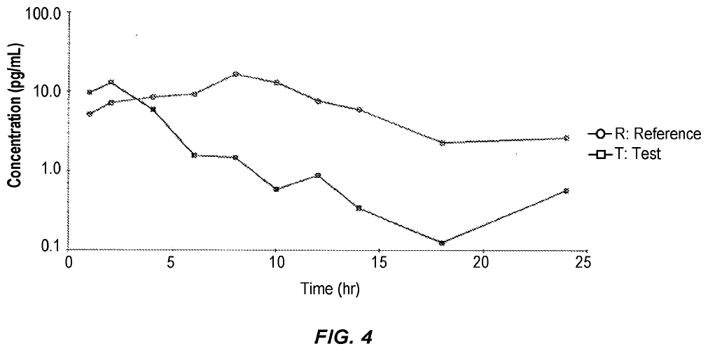

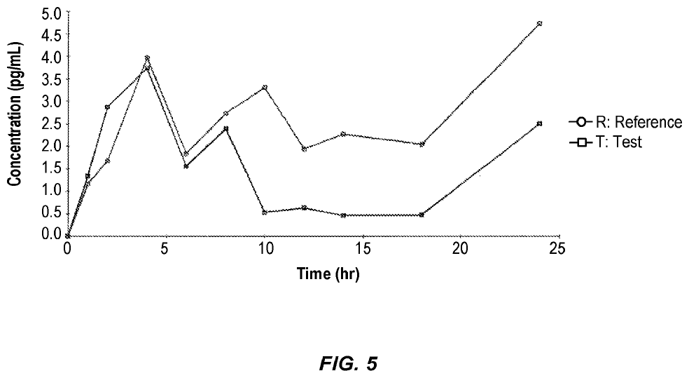

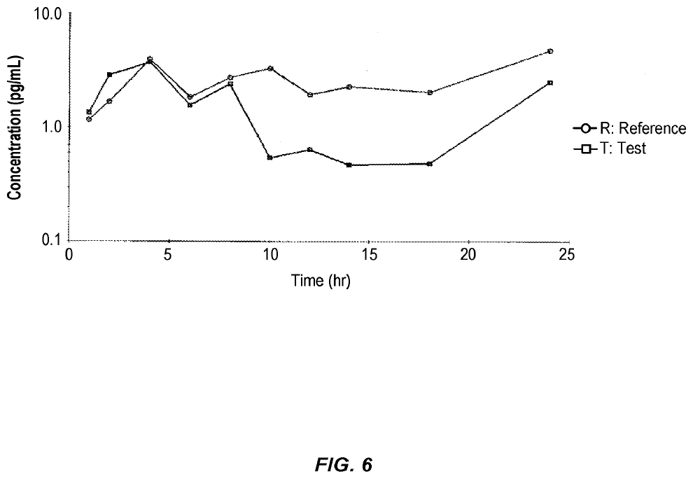

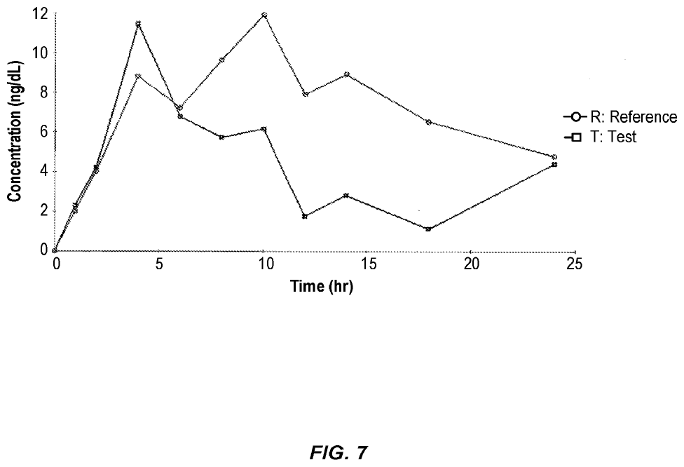

With reference to FIG. 2, delivery vehicle 200 includes pharmaceutical composition 202 and capsule 204. Width 208 represents the thickness of capsule 204, for example about 0.108 inches. The distance from one end of delivery vehicle 200 to another is represented by distance 206, for example about 0.690 inches. The size of delivery vehicle 200 may also be described by the arc swept by a radius of a given length. For example, arc 210, which is defined by the exterior of gelatin 204, is an arc swept by a radius of about 0.189 inches. Arc 212, which is defined by the interior of capsule 204, is an arc swept by a radius of about 0.0938 inches. Arc 214, which is defined by the exterior of gelatin 204 opposite arc 210, is an arc swept by a radius of about 0.108 inches. Suitable capsules of other dimensions may be provided. According to embodiments, capsule 204 has dimensions the same as or similar to the ratios as provided above relative to each other. In some embodiment, the gelatin capsule further comprises one or more components selected from the group consisting of hydrolyzed gelatin, sorbitol-sorbitan solution, water, glycerin, titanium dioxide, FD&C Red #40, ethanol, ethyl acetate, propylene glycol, polyvinyl acetate phthalate, isopropyl alcohol, polyethylene glycol, and ammonium hydroxide.

According to embodiments, the delivery vehicle is designed to remaining in the vagina until the pharmaceutical compositions are released. According to embodiments, delivery vehicle dissolves intravaginally and is absorbed into the vaginal tissue with the pharmaceutical composition, which minimizes vaginal discharge. In such embodiments, delivery mechanism is made from constituents that are non-toxic, for example, gelatin.

### Design Factors for Vaginally Inserted Pharmaceutical Compositions

According to embodiments, the pharmaceutical composition is designed to maximize favorable characteristics that lead to patient compliance (patients that discontinue treatment prior to completion of the prescribed course of therapy), without sacrificing efficacy. Favorable characteristics include, for example, lack of or reduction of irritation relative to other hormone replacement pessaries, lack of or reduction in vaginal discharge of the pharmaceutical composition and delivery vehicle relative to other hormone replacement pessaries, lack of or reduction of pharmaceutical composition or delivery vehicle residue inside the vagina, ease of administration compared to other hormone replacement pessaries, or improved efficacy of drug product relative to otherwise similar pharmaceutical compositions.

According to embodiments, the pharmaceutical composition is non-irritating or minimizes irritation. Patient irritation includes pain, pruritus (itching), soreness, excessive discharge, swelling, or other similar conditions. Patient irritation results in poor compliance. Non-irritating or reduced irritation pharmaceutical compositions are measured relative to competing hormone pessaries, including tablets, creams, or other intravaginal estrogen delivery forms.

According to embodiments, the pharmaceutical compositions does not result in systemic exposure (e.g., blood circulation of estradiol), which improves safety. According to other embodiments, the pharmaceutical compositions disclosed herein result in significantly reduced systemic exposure (e.g., blood circulation of estradiol) when compared to other vaginally administered drugs on the market for the treatment of VVA.

In certain embodiments, the administration of the pharmaceutical composition provides a mean concentration (Cₐᵥₑ) value below 20.6 pg/mL on Day 1 of the treatment, and/or a Cₐᵥₑ value below 19.4 pg/mL on Day 14 of the treatment, and/or a Cₐᵥₑ value below 11.5 pg/mL on Day 83 of the treatment. In certain embodiments, the administration of the pharmaceutical composition provides a mean concentration (Cₐᵥₑ) value below 10 pg/mL on Day 1 of the treatment, and/or a Cₐᵥₑ value below 7.3 pg/mL on Day 14 of the treatment, and/or a Cₐᵥₑ value below 5.5 pg/mL on Day 83 of the treatment.

According to embodiments, the pharmaceutical composition does not leave residue inside the vagina. Rather, the pharmaceutical composition and delivery vehicle are substantially absorbed or dispersed without resulting in unabsorbed residue or unpleasant sensations of non-absorbed or non-dispersed drug product. Measurement of lack of residue is relative to other vaginally inserted products or can be measured objectively with inspection of the vaginal tissues. For example, certain other vaginally inserted products contain starch which can result in greater discharge from the vagina following administration than. In some embodiments, the pharmaceutical compositions provided herein provide a lower amount, duration, or frequency of discharge following administration compared to other vaginally inserted products (e.g., compressed tablets).

According to embodiments, the pharmaceutical composition improves vaginal discharge compared to other pessaries, including pessaries that deliver hormones. Ideally, vaginal discharge is eliminated, minimized, or improved compared to competing products.

According to embodiments, the pharmaceutical compositions disclosed herein are inserted digitally. According to embodiments, the pharmaceutical compositions are digitally inserted approximately two inches into the vagina without a need for an applicator. According to embodiments, the pharmaceutical compositions are designed to be also inserted with an applicator, if desired. According to some embodiments, because the site of VVA is in the proximal region of the vagina (towards the vaginal opening), the pharmaceutical compositions disclosed herein are designed to be inserted in the proximal portion of the vagina.

Through extensive experimentation, various medium chain fatty acid esters of glycerol and propylene glycol demonstrated one or more favorable characteristics for development as a human drug product. According to embodiments, the solubilizing agent was selected from at least one of a solvent or co-solvent. Suitable solvents and co-solvents include any mono-, di- or triglyceride and glycols, and combinations thereof.

According to embodiments, the pharmaceutical composition is delivered via a gelatin capsule delivery vehicle. According to these embodiments, the pharmaceutical composition is a liquid pharmaceutical composition. According to embodiments, the delivery vehicle is a soft capsule, for example a soft gelatin capsule. Thus, the pharmaceutical composition of such embodiments is encapsulated in the soft gelatin capsule or other soft capsule.

According to embodiments, the pharmaceutical composition includes estradiol that is at least about 80% solubilized in a solubilizing agent comprising one or more C6 to C14 medium chain fatty acid mono-, di-, or triglycerides and, optionally, a thickening agent. According to embodiments, the pharmaceutical composition includes estradiol that is at least about 80% solubilized one or more C6 to C12 medium chain fatty acid mono-, di-, or triglycerides, e.g., one or more C6 to C14 triglycerides, e.g., one or more C6 to C12 triglycerides, such as one or more C8-C10 triglycerides. These embodiments specifically contemplate the estradiol being at least 80% solubilized. These embodiments specifically contemplate the estradiol being at least 90% solubilized. These embodiments specifically contemplate the estradiol being at least 95% solubilized. These embodiments specifically contemplate the estradiol being fully solubilized.

As noted above, liquid pharmaceutical compositions are liquid at room temperature or at body temperature. For example, in some embodiments, a pharmaceutical composition provided herein is a liquid formulation contained within a soft gel capsule. Gels, hard fats, or other solid forms that are not liquid at room or body temperature are less desirable in embodiments of the pharmaceutical composition that are liquid.

The thickening agent serves to increase viscosity, e.g., up to about 10,000 cP (10,000 mPa-s), typically to no more than about 5000 cP, and more typically to between about 50 and 1000 cP. In embodiments, the non-ionic surfactant, e.g., GELUCIRE or TEFOSE, may be solid at room temperature and require melting to effectively mix with the solubilizing agent. However, in these embodiments, the resultant pharmaceutical composition remains liquid, albeit with greater viscosity, not solid.

According to embodiments, the pharmaceutical composition includes estradiol, the medium chain solubilizing agent, and the thickening agent as the ingredients delivered via a soft capsule delivery vehicle. Other ingredients, e.g., colorants, antioxidants, preservatives, or other ingredients may be included as well. However, the addition of other ingredients should be in amounts that do not materially change the solubility of the estradiol, the pharmacokinetics of the pharmaceutical composition, or efficacy of the pharmaceutical composition. Other factors that should be considered when adjusting the ingredients of the pharmaceutical composition include the irritation, vaginal discharge, intravaginal residue, and other relevant factors, for example those that would lead to reduced patient compliance. Other contemplated ingredients include: oils or fatty acid esters, lecithin, mucoadherent agents, gelling agents, dispersing agents, or the like.

### VI. METHODS

According to embodiments, the pharmaceutical compositions disclosed herein can be used for the treatment of VVA, including the treatment of at least one VVA symptom including: vaginal dryness, vaginal or vulvar irritation or itching, dysuria, dyspareunia, and vaginal bleeding associated with sexual activity, among others. According to embodiments the methods of treatment are generally applicable to females.

According to embodiments, the pharmaceutical compositions disclosed herein can be used for the treatment of estrogen-deficient urinary states. According to embodiments, the pharmaceutical compositions disclosed herein can be used for the treatment of dyspareunia, or vaginal bleeding associated with sexual activity.

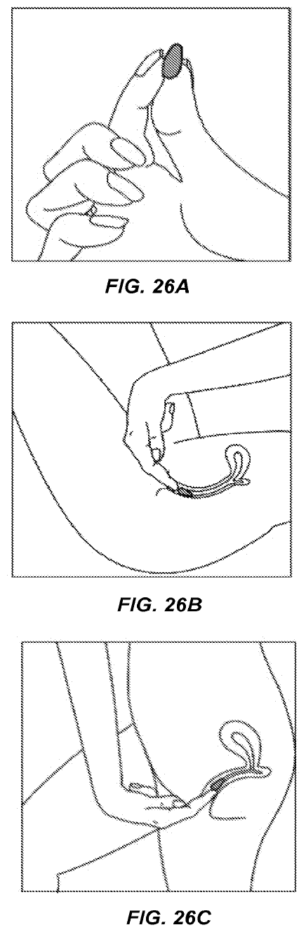

According to embodiments, treatment of the VVA, estrogen-deficient urinary states, and dyspareunia and vaginal bleeding associated with sexual activity occurs by administering the pharmaceutical compositions intravaginally. According to embodiments where the delivery vehicle is a capsule, the patient obtains the capsule and inserts the capsule into the vagina, where the capsule dissolves and the pharmaceutical composition is released into the vagina where it is absorbed into the vaginal tissue. In some embodiments, the pharmaceutical composition is completely absorbed into the vaginal tissue. In some embodiments, the pharmaceutical composition is substantially absorbed into the vaginal tissue (e.g., at least about 80% by weight, at least about 85% by weight, at least about 90% by weight, at least about 95% by weight, at least about 97% by weight, at least about 98% by weight, or at least about 99% by weight of the composition is absorbed). According to embodiments, the capsule is inserted about two inches into the vagina, however the depth of insertion is generally any depth that allows for adsorption of substantially all of the pharmaceutical composition. According to embodiments, the capsule can also be applied using an applicator that deposits the capsule at an appropriate vaginal depth as disclosed herein. According to embodiments, the capsule is inserted into the lower third of the vagina (i.e., the third closest to the vaginal opening) or about two inches into the vagina. According to embodiments, the softgel capsule can be held with the larger end between the fingers as shown in FIG. 26A.

The subject will select a position that is most comfortable (e.g., a reclining position as shown in FIG. 26B or a standing position as shown in FIG. 26C), and the subject will insert the softgel into the lower third of the vagina or about two inches into the vagina with the smaller end up. The softgel capsule will dissolve rapidly. The softgel can be inserted at any time of day and normal activities can be immediately resumed. According to embodiments, the same time of day for all insertions of of the softgel is used.

According to embodiments where the pharmaceutical composition is a cream, gel, ointment, or other similar preparation, the pharmaceutical composition is applied digitally, as is well known and understood in the art.

Upon release of the pharmaceutical composition in the vagina, estradiol is locally absorbed. For example, following administration of the suppository to the proximal region of the vagina of a patient provides a therapeutically effective concentration of estradiol over 24 hours in the proximal region of the vagina.

According to embodiments, the timing of administration of the pharmaceutical composition of this disclosure may be conducted by any safe means as prescribed by an attending physician. According to embodiments, a patient will administer the pharmaceutical composition (e.g., a capsule) intravaginally each day for 14 days, then twice weekly thereafter. In some such embodiments, the doses administered during the twice weekly dosing period are administered approximately 3-4 days apart. Typically, doses administered during the twice weekly dosing period do not exceed more than twice in a seven day period.

According to embodiments, a patient vaginally administers the estradiol (e.g., the pharmaceutical composition comprising estradiol, e.g., a capsule) once daily for two weeks, then twice weekly thereafter. In some embodiments, the estradiol is administered at a dosage of 4 mcg. In some embodiments, the estradiol is administered at a starting dosage of 4 mcg, and dosage may be adjusted based on the patient's clinical response. In some embodiments, the estradiol is administered at a starting dosage of 4 mcg, and the dosage is increased if the patient does not exhibit an improvement in a clinical response. In some embodiments, the estradiol is administered at a starting dosage of 4 mcg, and the dosage is increased to 4.5 mcg, 5 mcg, 6 mcg, 7 mcg, 8 mcg, 9 mcg, 10 mcg, 11 mcg, 12 mcg, 13 mcg, 14 mcg, 15 mcg, 16 mcg, 17 mcg, 18 mcg, 19 mcg, 20 mcg, 21 mcg, 22 mcg, 22 mcg, 24 mcg, or 25 mcg if the patient does not exhibit an improvement in a clinical response. In some embodiments, the estradiol is administered at a starting dosage of 4 mcg, and the dosage is increased to 10 mcg if the patient does not exhibit an improvement in a clinical response. In some embodiments, the dosage may be further adjusted (e.g., decreased or further increased) based on the patient's clinical response.

In some embodiments, the estradiol is administered at a starting dosage of 4 mcg, and the dosage is maintained or decreased if the patient exhibits an improvement in a clinical response. In some embodiments, the estradiol is administered at a starting dosage of 4 mcg, and the 4 mcg dosage is maintained if the patient exhibits an improvement in a clinical response. In some embodiments, the estradiol is administered at a starting dosage of 4 mcg, and the dosage is decreased to 3.5 mcg, 3 mcg, 2.5 mcg, or 2 mcg if the patient exhibits an improvement in a clinical response. In some embodiments, the starting dosage or the decreased dosage is maintained for a defined period of time or indefinitely. In some embodiments, the starting dosage or decreased dosage is maintained for as long as the dosage is efficacious (e.g., in treating VVA).

In some embodiments, the estradiol is administered at a starting dosage of 10 mcg, and the dosage is maintained or decreased if the patient exhibits an improvement in a clinical response. In some embodiments, the estradiol is administered at a starting dosage of 10 mcg, and the 10 mcg dosage is maintained if the patient exhibits an improvement in a clinical response. In some embodiments, the estradiol is administered at a starting dosage of 10 mcg, and the dosage is decreased to 9 mcg, 8 mcg, 7 mcg, 6 mcg, 5 mcg, 4 mcg, 3 mcg, or 2.5 mcg if the patient exhibits an improvement in a clinical response. It will be appreciated by a person of ordinary skill in the art that it can be desirable to administer the lowest possible efficacious dosage of estradiol (e.g., the lowest possible dosage of estradiol that is efficacious in treating VVA). In a preferred embodiment, the dosage is decreased from 10 mcg to 4 mcg if the patient exhibits an improvement in a clinical response. In some embodiments, the starting dosage or the decreased dosage is maintained for a defined period of time or indefinitely. In some embodiments, the starting dosage or decreased dosage is maintained for as long as the dosage is efficacious (e.g., in treating VVA).

In some embodiments, the estradiol is administered at a starting dosage of 25 mcg, and the dosage is maintained or decreased if the patient exhibits an improvement in a clinical response. In some embodiments, the estradiol is administered at a starting dosage of 25 mcg, and the 25 mcg dosage is maintained if the patient exhibits an improvement in a clinical response. In some embodiments, the estradiol is administered at a starting dosage of 25 mcg, and the dosage is decreased to 20 mcg, 18 mcg, 16 mcg, 15 mcg, 14 mcg, 12 mcg, or 10 mcg if the patient exhibits an improvement in a clinical response. It will be appreciated by a person of ordinary skill in the art that it can be desirable to administer the lowest possible efficacious dosage of estradiol (e.g., the lowest possible dosage of estradiol that is efficacious in treating VVA). In a preferred embodiment, the dosage is decreased from 25 mcg to 10 mcg if the patient exhibits an improvement in a clinical response. In another preferred embodiment, the decreased dosage (e.g., 10 mcg) is further decreased after a period of time if the patient receiving the decreased dosage exhibits an improvement in a clinical response. In some embodiments, the dosage is decreased to less than 10 mcg, e.g., to 9 mcg, 8 mcg, 7 mcg, 6 mcg, 5 mcg, 4 mcg, 3 mcg, or 2.5 mcg if the patient exhibits an improvement in a clinical response. In a preferred embodiment, the dosage is decreased from a starting dosage of 25 mcg to 10 mcg, and then from a decreased dosage of 10 mcg to 4 mcg. In some embodiments, the starting dosage or the decreased dosage is maintained for a defined period of time or indefinitely. In some embodiments, the starting dosage or decreased dosage is maintained for as long as the dosage is efficacious (e.g., in treating VVA).

According to embodiments, the pharmaceutical compositions are vaginally administered with co-administration of an orally administered estrogen-based (or progestin-based or progestin- and estrogen-based) pharmaceutical drug product, or patch, cream, gel, spray, transdermal delivery system or other parenterally-administered estrogen-based pharmaceutical drug product, each of which can include natural, bio-similar, or synthetic or other derived estrogens or progestins. According to embodiments, modulation of circulating estrogen levels provided via the administration of the pharmaceutical compositions disclosed herein, if any, are not intended to be additive to any co-administered estrogen product and its associated circulating blood levels. According to other embodiments, co-administrated estrogen products are intended to have an additive effect as would be determined by the patient physician.

According to embodiments, a method for estrogenizing vaginal tissue is provided. The method includes administration of a (i.e., a suppository) or dosage as described herein Estrogenized vaginal tissue is typically characterized by one or more of the following properties: the presence clear secretions on vaginal walls; rogation and elasticity of the vaginal walls; intact vaginal epithelium; and pink tissue color. In contrast, de-estrogenized vaginal is characterized by decreased or absent secretions; smooth tissue with fewer or no rugae; bleeding of the vaginal surface; development of petechiae (i.e., pinpoint, round spots on the skin due to bleeding, appearing red, brown, or purple); and pale or transparent tissues. Accordingly, estrogenizing vaginal tissue according to the method disclosed herein can include, increasing the level of vaginal secretions in a subject; increasing the number of vaginal rugae in the subject; and/or decreasing bleeding or petechiae in the subject. According to embodiments, a method for estrogenizing vaginal tissue is provided, the method including administering a suppository so as to provide an estradiol Cₘₐₓ or AUC as described herein. According to embodiments, a method for estrogenizing vaginal tissue is provided, the method including administering a suppository so as to provide an estrone Cₘₐₓ or AUC as described herein.

According to embodiments, a method for estrogenizing the labia majora and labia minora (collectively “labia”) is provided as described herein. Generally, the pharmaceutical composition is inserted digitally into the vagina approximately two inches or inserted into the third of the vagina closest to the vaginal opening as shown in FIGS. 26A, 26B, and 26C. The gelatin capsule containing the pharmaceutical composition dissolves, ruptures, or otherwise releases the pharmaceutical composition into the vagina, whereby the lower third of the vagina and labia are both reestrogenized. According to some embodiments, the pharmaceutical composition is a liquid that partially flows to the labia and directly reestrogenizes the labia.

According to embodiments, a method for estrogenizing the vulva is provided as described herein. Generally, the pharmaceutical composition is inserted digitally into the vagina approximately two inches or inserted into the third of the vagina closest to the vaginal opening as shown in FIGS. 26A, 26B, and 26C. The gelatin capsule containing the pharmaceutical composition dissolves, ruptures, or otherwise releases the pharmaceutical composition into the vagina, whereby the lower third of the vagina and vulva are both reestrogenized. According to some embodiments, the pharmaceutical composition is a liquid that partially flows to the vulval tissue and directly reestrogenizes the vulva.

According to embodiments, a method for treating vaginal dryness is provided. The method includes administration of a soft gel vaginal estradiol formulation (i.e., a suppository) or dosage as described herein. Treating vaginal dryness according to the method disclosed herein can include, decreasing the severity of vaginal dryness by 1%, 5%, 10%, 15%, 20%, 25%, 30%, 45% 50%, 55%, 60%, 65%, 70%, 75%, 80%, 85%, 90%, 95% or 99%. The decrease in severity can be obtained following 2 weeks of treatment, or 6 weeks of treatment, or 8 weeks of treatment, or 12 weeks of treatment. In some embodiments, vaginal dryness is assessed using a severity scale, ranging from 0 to 4 points wherein 0 indicates no dryness, 1 indicates mild dryness, 2 indicates moderate dryness, and 3 indicates severe dryness.

In some embodiments, the method for treating vaginal dryness includes reducing the dryness severity score from 3, prior to treatment of a subject, to 2, after 2 weeks of treatment of the subject. In some embodiments, the method for treating vaginal dryness includes reducing the dryness severity score from 2, prior to treatment of a subject, to 1, after 2 weeks of treatment of the subject. In some embodiments, the method for treating vaginal dryness includes reducing the dryness severity score from 1, prior to treatment of subject, to 0, after 2 weeks of treatment of the subject.

In some embodiments, the method for treating vaginal dryness includes reducing the dryness severity score from 3, prior to treatment of a subject, to 2, after 6 weeks of treatment of the subject. In some embodiments, the method for treating vaginal dryness includes reducing the dryness severity score from 2, prior to treatment of a subject, to 1, after 6 weeks of treatment of the subject. In some embodiments, the method for treating vaginal dryness includes reducing the dryness severity score from 1, prior to treatment of subject, to 0, after 6 weeks of treatment of the subject.

In some embodiments, the method for treating vaginal dryness includes reducing the dryness severity score from 3, prior to treatment of a subject, to 2, after 8 weeks of treatment of the subject. In some embodiments, the method for treating vaginal dryness includes reducing the dryness severity score from 2, prior to treatment of a subject, to 1, after 8 weeks of treatment of the subject. In some embodiments, the method for treating vaginal dryness includes reducing the dryness severity score from 1, prior to treatment of subject, to 0, after 8 weeks of treatment of the subject.

In some embodiments, the method for treating vaginal dryness includes reducing the dryness severity score from 3, prior to treatment of a subject, to 2, after 12 weeks of treatment of the subject. In some embodiments, the method for treating vaginal dryness includes reducing the dryness severity score from 2, prior to treatment of a subject, to 1, after 12 weeks of treatment of the subject. In some embodiments, the method for treating vaginal dryness includes reducing the dryness severity score from 1, prior to treatment of subject, to 0, after 12 weeks of treatment of the subject.

In some embodiments, the method for treating vaginal dryness includes decreasing the severity of dryness after two weeks of treatment, wherein the severity is assessed on a scale of 0-3 points, and the average decrease ranges from a 0.5-point decrease to a 1.25-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after six weeks of treatment, wherein the severity is assessed on a scale of 0-3 points, and the average decrease ranges from a 0.75-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after eight weeks of treatment, wherein the severity is assessed on a scale of 0-3 points, and the average decrease ranges from a 0.9-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes decreasing the severity of dryness after twelve weeks of treatment, wherein the severity is assessed on a scale of 0-3 points, and the average decrease ranges from a 0.9-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vaginal dryness includes administering a suppository so as to provide an estradiol Cₘₐₓ or AUC as described herein. According to embodiments, a method for treating vaginal dryness is provided, the method including administering a suppository so as to provide an estrone Cₘₐₓ or AUC as described herein.

According to embodiments, a method for treating vulvar and/or vaginal itching or irritation is provided. The method includes administration of a soft gel vaginal estradiol formulation (i.e., a suppository) or dosage as described herein. Treating vulvar and/or vaginal itching or irritation according to the method disclosed herein can include, decreasing the severity of vulvar and/or vaginal itching or irritation by 1%, 5%, 10%, 15%, 20%, 25%, 30%, 45%, 50%, 55%, 60%, 65%, 70%, 75%, 80%, 85%, 90%, 95%, or 99%. The decrease in severity can be obtained following 2 weeks of treatment, or 6 weeks of treatment, or 8 weeks of treatment, or 12 weeks of treatment. In some embodiments, vulvar and/or vaginal itching or irritation is assessed using a severity scale, ranging from 0 to 4 points wherein 0 indicates no itching or irritation, 1 indicates mild itching or irritation, 2 indicates moderate itching or irritation, and 3 indicates severe itching or irritation.

In some embodiments, the method for treating vulvar and/or vaginal itching or irritation includes reducing the itching/irritation severity score from 3, prior to treatment of a subject, to 2, after 2 weeks of treatment of the subject. In some embodiments, the method for treating vulvar and/or vaginal itching or irritation includes reducing the itching/irritation severity score from 2, prior to treatment of a subject, to 1, after 2 weeks of treatment of the subject. In some embodiments, the method for treating vulvar and/or vaginal itching or irritation includes reducing the itching/irritation severity score from 1, prior to treatment of subject, to 0, after 2 weeks of treatment of the subject.

In some embodiments, the method for treating vulvar and/or vaginal itching or irritation includes reducing the itching/irritation severity score from 3, prior to treatment of a subject, to 2, after 6 weeks of treatment of the subject. In some embodiments, the method for treating vulvar and/or vaginal itching or irritation includes reducing the itching/irritation severity score from 2, prior to treatment of a subject, to 1, after 6 weeks of treatment of the subject. In some embodiments, the method for treating vulvar and/or vaginal itching or irritation includes reducing the itching/irritation severity score from 1, prior to treatment of subject, to 0, after 6 weeks of treatment of the subject.

In some embodiments, the method for treating vulvar and/or vaginal itching or irritation includes reducing the itching/irritation severity score from 3, prior to treatment of a subject, to 2, after 8 weeks of treatment of the subject. In some embodiments, the method for treating vulvar and/or vaginal itching or irritation includes reducing the itching/irritation severity score from 2, prior to treatment of a subject, to 1, after 8 weeks of treatment of the subject. In some embodiments, the method for treating vulvar and/or vaginal itching or irritation includes reducing the itching/irritation severity score from 1, prior to treatment of subject, to 0, after 8 weeks of treatment of the subject.

In some embodiments, the method for treating vulvar and/or vaginal itching or irritation includes reducing the itching/irritation severity score from 3, prior to treatment of a subject, to 2, after 12 weeks of treatment of the subject. In some embodiments, the method for treating vulvar and/or vaginal itching or irritation includes reducing the itching/irritation severity score from 2, prior to treatment of a subject, to 1, after 12 weeks of treatment of the subject. In some embodiments, the method for treating vulvar and/or vaginal itching or irritation includes reducing the itching/irritation severity score from 1, prior to treatment of subject, to 0, after 12 weeks of treatment of the subject.

In some embodiments, the method for treating vulvar and/or vaginal itching or irritation includes decreasing the severity of itching/irritation after two weeks of treatment, wherein the severity is assessed on a scale of 0-3 points, and the average decrease ranges from a 0.3-point decrease to a 0.6-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after six weeks of treatment, wherein the severity is assessed on a scale of 0-3 points, and the average decrease ranges from a 0.5-point decrease to a 0.7-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after eight weeks of treatment, wherein the severity is assessed on a scale of 0-3 points, and the average decrease ranges from a 0.5-point decrease to a 0.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes decreasing the severity of itching/irritation after twelve weeks of treatment, wherein the severity is assessed on a scale of 0-3 points, and the average decrease ranges from a 0.5-point decrease to a 1.0-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating vulvar and/or vaginal itching or irritation includes administering a suppository so as to provide an estradiol Cₘₐₓ or AUC as described herein. According to embodiments, a method for treating vulvar and/or vaginal itching or irritation is provided, the method including administering a suppository so as to provide an estrone Cₘₐₓ or AUC as described herein.

According to embodiments, a method for treating dyspareunia is provided. The method includes administration of a suppository or dosage as described herein. Treating dyspareunia according to the method disclosed herein can include, decreasing the severity of dyspareunia by 1%, 5%, 10%, 15%, 20%, 25%, 30%, 45%, 50%, 55%, 60%, 65%, 70%, 75%, 80%, 85%, 90%, 95%, or 99%. The decrease in severity can be obtained following 2 weeks of treatment, or 6 weeks of treatment, or 8 weeks of treatment, or 12 weeks of treatment. In some embodiments, dyspareunia is assessed using a severity scale, ranging from 0 to 4 points wherein 0 indicates no pain associated with sexual activity (with vaginal penetration), 1 indicates mild pain associated with sexual activity (with vaginal penetration), 2 indicates moderate pain associated with sexual activity (with vaginal penetration), and 3 indicates severe pain associated with sexual activity (with vaginal penetration).

In some embodiments, the method for treating dyspareunia includes reducing the dyspareunia severity score from 3, prior to treatment of a subject, to 2, after 2 weeks of treatment of the subject. In some embodiments, the method for treating dyspareunia includes reducing the dyspareunia severity score from 2, prior to treatment of a subject, to 1, after 2 weeks of treatment of the subject. In some embodiments, the method for treating dyspareunia includes reducing the dyspareunia severity score from 1, prior to treatment of subject, to 0, after 2 weeks of treatment of the subject.

In some embodiments, the method for treating dyspareunia includes reducing the dyspareunia severity score from 3, prior to treatment of a subject, to 2, after 6 weeks of treatment of the subject. In some embodiments, the method for treating dyspareunia includes reducing the dyspareunia severity score from 2, prior to treatment of a subject, to 1, after 6 weeks of treatment of the subject. In some embodiments, the method for treating dyspareunia includes reducing the dyspareunia severity score from 1, prior to treatment of subject, to 0, after 6 weeks of treatment of the subject.

In some embodiments, the method for treating dyspareunia includes reducing the dyspareunia severity score from 3, prior to treatment of a subject, to 2, after 8 weeks of treatment of the subject. In some embodiments, the method for treating dyspareunia includes reducing the dyspareunia severity score from 2, prior to treatment of a subject, to 1, after 8 weeks of treatment of the subject. In some embodiments, the method for treating dyspareunia includes reducing the dyspareunia severity score from 1, prior to treatment of subject, to 0, after 8 weeks of treatment of the subject.

In some embodiments, the method for treating dyspareunia includes reducing the dyspareunia severity score from 3, prior to treatment of a subject, to 2, after 12 weeks of treatment of the subject. In some embodiments, the method for treating dyspareunia includes reducing the dyspareunia severity score from 2, prior to treatment of a subject, to 1, after 12 weeks of treatment of the subject. In some embodiments, the method for treating dyspareunia includes reducing the dyspareunia severity score from 1, prior to treatment of subject, to 0, after 12 weeks of treatment of the subject.

In some embodiments, the method for treating dyspareunia includes decreasing the severity of dyspareunia after two weeks of treatment, wherein the severity is assessed on a scale of 0-3 points, and the average decrease ranges from a 0.9-point decrease to a 1.1-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after six weeks of treatment, wherein the severity is assessed on a scale of 0-3 points, and the average decrease ranges from a 1.3-point decrease to a 1.5-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after eight weeks of treatment, wherein the severity is assessed on a scale of 0-3 points, and the average decrease ranges from a 1.5-point decrease to a 1.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes decreasing the severity of dyspareunia after twelve weeks of treatment, wherein the severity is assessed on a scale of 0-3 points, and the average decrease ranges from a 1.5-point decrease to a 1.8-point decrease. The average decrease can be determined by observing any suitable number of subjects. In some embodiments, the number of subjects is at least 100. In some embodiments, the number of subjects is at least 500. In some embodiments, the number of subjects ranges from 700 to 800. In some embodiments, the number of subjects ranges from 740 to 750. In some embodiments, the vaginal estradiol formulation contains 4 μg of estradiol. In some embodiments, the vaginal estradiol formulation contains 10 μg of estradiol. In some embodiments, the vaginal estradiol formulation contains 25 μg of estradiol.

In some embodiments, the method for treating dyspareunia includes administering a suppository so as to provide an estradiol Cₘₐₓ or AUC as described herein. According to embodiments, a method for treating dyspareunia is provided, the method including administering a suppository so as to provide an estrone Cₘₐₓ or AUC as described herein.

According to embodiments, a method for treating urinary tract infections is provided. As used herein the term “urinary tract infection” refers to an infection of the kidneys, ureters, bladder and urethra by a microorganism such as Escherichia coli, Staphylococcus saprophyticus, Klebsiella sp., Enterobacter sp., or Proteus sp. The method for treating urinary tract infections generally includes administering a soft gel vaginal estradiol formulation (i.e., a suppository) as described herein. According to certain embodiments, the method further includes decreasing urethral discomfort, frequency or urination, hematuria, dysuria, and/or stress incontinence. According to certain embodiments, a method for treating urinary tract infections is provided, the method including administering a suppository as described herein and decreasing vaginal pH from above 4.5 to between 3.5 and 4.5 (inclusive). The method can be particularly effective for treating urinary tract infections in elderly subjects (e.g., subjects older than 65 years, or older than 75 years, or older than 85 years). According to embodiments, a method for treating urinary tract infections is provided, the method including administering a suppository so as to provide an estradiol Cₘₐₓ or AUC as described herein. According to embodiments, a method for treating urinary tract infections is provided, the method including administering a suppository so as to provide an estrone Cₘₐₓ or AUC as described herein. According to embodiments, a method for treating sexual dysfunction is provided. As used herein with respect to female subjects, the term “sexual dysfunction” generally refers to pain or discomfort during sexual intercourse, diminished vaginal lubrication, delayed vaginal engorgement, increased time for arousal, diminished ability to reach orgasm, diminished clitoral sensation, diminished sexual desire, and/or diminished arousal. According to embodiments, a method for treating sexual dysfunction is provided, the method including administering a suppository so as to provide an estradiol Cₘₐₓ or AUC as described herein. According to embodiments, a method for treating sexual dysfunction is provided, the method including administering a suppository so as to provide an estrone Cₘₐₓ or AUC as described herein.

Sexual function and dysfunction can be assessed using the Female Sexual Function Index (FSFI) (see, Rosen R, Brown C, Heiman J, et al. “The Female Sexual Function Index (FSFI): A Multidimensional Self-Report Instrument for the Assessment of Female Sexual Function.” Journal of Sex & Marital Therapy 2000. 26: p. 191-208). The FSFI is useful for assessing various domains of sexual functioning (e.g. sexual desire, arousal, orgasm, satisfaction and pain). Accordingly, the method for treating sexual dysfunction as provided herein can include administering a vaginal soft gel formulation to a subject and increasing a subject's full-scale FSFI score, FSFI-desire score, FSFI-arousal score, FSFI-lubrication score and/or FSFI-orgasm score.

Female Sexual Function Index (FSFI)

| Question | Answer Options |
|---|---|
| Q1: Over the past 4 weeks, | 5 = Almost always or always |
| how often did you feel | 4 = Most times (more than half the time) |
| sexual desire or interest? | 3 = Sometimes (about half the time) |
| | 2 = A few times (less than half the time) |
| | 1 = Almost never or never |
| Q2: Over the past 4 weeks, | 5 = Very high |
| how would you rate your | 4 = High |
| level (degree) of sexual | 3 = Moderate |
| desire or interest? | 2 = Low |
| | 1 = Very low or none at all |
| Q3. Over the past 4 weeks, | 0 = No sexual activity |
| how often did you feel | 5 = Almost always or always |
| sexually aroused (“turned | 4 = Most times (more than half the time) |
| on”) during sexual activity | 3 = Sometimes (about half the time) |
| or intercourse? | 2 = A few times (less than half the time) |
| | 1 = Almost never or never |
| Q4. Over the past 4 weeks, | 0 = No sexual activity |
| how would you rate your | 5 = Very high |
| level of sexual arousal (“turn | 4 = High |
| on”) during sexual activity | 3 = Moderate |
| or intercourse? | 2 = Low |
| | 1 = Very low or none at all |
| Q5. Over the past 4 weeks, | 0 = No sexual activity |
| how confident were you | 5 = Very high confidence |
| about becoming sexually | 4 = High confidence |
| aroused during sexual | 3 = Moderate confidence |
| activity or intercourse? | 2 = Low confidence |
| | 1 = Very low or no confidence |
| Q6. Over the past 4 weeks, | 0 = No sexual activity |
| how often have you been | 5 = Almost always or always |
| satisfied with your arousal | 4 = Most times (more than half the time) |
| (excitement) during sexual | 3 = Sometimes (about half the time) |
| activity or intercourse? | 2 = A few times (less than half the time) |
| Response Options | 1 = Almost never or never |
| Q7: Over the past 4 weeks, | 0 = No sexual activity |
| how often did you become | 5 = Almost always or always |
| lubricated (“wet”) during | 4 = Most times (more than half the time) |
| sexual activity or | 3 = Sometimes (about half the time) |
| intercourse? | 2 = A few times (less than half the time) |
| | 1 = Almost never or never |
| Q8. Over the past 4 weeks, | 0 = No sexual activity |
| how difficult was it to | 1 = Extremely difficult or impossible |
| become lubricated (“wet”) | 2 = Very difficult |
| during sexual activity or | 3 = Difficult |
| intercourse? | 4 = Slightly difficult |
| | 5 = Not difficult |
| Q9: Over the past 4 weeks, | 0 = No sexual activity |
| how often did you maintain | 5 = Almost always or always |
| your lubrication (“wetness”) | 4 = Most times (more than half the time) |
| until completion of sexual | 3 = Sometimes (about half the time) |
| activity or intercourse? | 2 = A few times (less than half the time) |
| | 1 = Almost never or never |
| Q10: Over the past 4 weeks, | 0 = No sexual activity |
| how difficult was it to | 1 = Extremely difficult or impossible |
| maintain your lubrication | 2 = Very difficult |
| (“wetness”) until completion | 3 = Difficult |
| of sexual activity or | 4 = Slightly difficult |
| intercourse? | 5 = Not difficult |
| Q11. Over the past 4 weeks, | 0 = No sexual activity |
| when you had sexual | 5 = Almost always or always |
| stimulation or intercourse, | 4 = Most times (more than half the time) |
| how often did you reach | 3 = Sometimes (about half the time) |
| orgasm (climax)? | 2 = A few times (less than half the time) |
| | 1 = Almost never or never |
| Q12: Over the past 4 weeks, | 0 = No sexual activity |
| when you had sexual | 1 = Extremely difficult or impossible |
| stimulation or intercourse, | 2 = Very difficult |
| how difficult was it for you | 3 = Difficult |
| to reach orgasm (climax)? | 4 = Slightly difficult |
| | 5 = Not difficult |
| Q13: Over the past 4 weeks, | 0 = No sexual activity |
| how satisfied were you with | 5 = Very satisfied 4 |
| your ability to reach orgasm | 4 = Moderately satisfied |
| (climax) during sexual | 3 = About equally satisfied and dissatisfied |
| activity or intercourse? | 2 = Moderately dissatisfied |
| | 1 = Very dissatisfied |
| Q14: Over the past 4 weeks, | 0 = No sexual activity |
| how satisfied have you been | 5 = Very satisfied |
| with the amount of | 4 = Moderately satisfied |
| emotional closeness during | 3 = About equally satisfied and dissatisfied |
| sexual activity between you | 2 = Moderately dissatisfied |
| and your partner? | 1 = Very dissatisfied |
| Q15: Over the past 4 weeks, | 5 = Very satisfied |
| how satisfied have you been | 4 = Moderately satisfied |
| with your sexual | 3 = About equally satisfied and dissatisfied |
| relationship with your | 2 = Moderately dissatisfied |
| partner? | 1 = Very dissatisfied |
| Q16: Over the past 4 weeks, | 5 = Very satisfied |
| how satisfied have you been | 4 = Moderately satisfied |
| with your overall sexual | 3 = About equally satisfied and dissatisfied |
| life? | 2 = Moderately dissatisfied |
| | 1 = Very dissatisfied |
| Q17: Over the past 4 weeks, | 0 = Did not attempt intercourse |
| how often did you | 1 = Almost always or always |
| experience discomfort or | 2 = Most times (more than half the time) |
| pain during vaginal | 3 = Sometimes (about half the time) |
| penetration? | 4 = A few times (less than half the time) |
| | 5 = Almost never or never |
| Q18: Over the past 4 weeks, | 0 = Did not attempt intercourse |
| how often did you | 1 = Almost always or always |
| experience discomfort or | 2 = Most times (more than half the time) |
| pain following vaginal | 3 = Sometimes (about half the time) |
| penetration? | 4 = A few times (less than half the time) |
| | 5 = Almost never or never |
| Q19. Over the past 4 weeks, | 0 = Did not attempt intercourse |
| how would you rate your | 1 = Very high |
| level (degree) of discomfort | 2 = High |
| or pain during or following | 3 = Moderate |
| vaginal penetration? | 4 = Low |
| | 5 = Very low or none at all |

FSFI Scoring System

| Domain | Questions | Score Range | Factor | Minimum | Maximum |
|---|---|---|---|---|---|
| Desire | 1, 2 | 1-5 | 0.6 | 1.2 | 6.0 |
| Arousal | 3, 4, 5, 6 | 0-5 | 0.3 | 0 | 6.0 |
| Lubrication | 7, 8, 9, 10 | 0-5 | 0.3 | 0 | 6.0 |
| Orgasm | 11, 12, 13 | 0-5 | 0.4 | 0 | 6.0 |
| Satisfaction | 14, 15, 16 | 0 (or 1)-5 | 0.4 | 0.8 | 6.0 |
| Pain | 17, 18, 19 | 0-5 | 0.4 | 0 | 6.0 |

| | | |
|---|---|---|
| Full Scale Score Range: | 2.0 | 36.0 |

In some embodiments, the method for treating sexual dysfunction includes administering estradiol to the subject and increasing the FSFI-desire score by at least about 20%, or at least about 25%, or at least about 30% as compared to baseline.

In some embodiments, the method for treating sexual dysfunction includes administering estradiol to the subject and increasing the FSFI-arousal score by at least about 30%, or at least about 40%, or at least about 50% as compared to baseline.

In some embodiments, the method for treating sexual dysfunction includes administering estradiol to the subject and increasing the FSFI-lubrication score by at least about 85%, or at least about 95%, or at least about 115% as compared to baseline.

In some embodiments, the method for treating sexual dysfunction includes administering estradiol to the subject and increasing the FSFI-orgasm score by at least about 40%, or at least about 60% as compared to baseline.

In some embodiments, the method for treating sexual dysfunction includes administering estradiol to the subject and increasing the total FSFI score by at least about 50%, or at least about 55%, or at least about 70% as compared to baseline.

Examples of other metrics for assessment of sexual function include, but are not limited to, Changes in Sexual Function Questionnaire (“CSFQ”; Clayton et al., Psychopharmacol Bull. 33(4):731-45 (1997) and Clayton et al., Psychopharmacol. Bull. 33(4):747-53 (1997)); the Derogatis Interview for Sexual Functioning—Self-Report (“DISF-SR”; Derogatis, J Sex Marital Ther. 23:291-304 (1997)); the Golombok-Rust Inventory of Sexual Satisfaction (“GRISS”; Rust et al., Arch. Sex Behav. 15:157-165 (1986)); the Sexual Function Questionnaire (“SFQ”; Quirk et al., J Womens Health GendBasedMed. 11:277-289 (2002)); and the Arizona Sexual Experience Scale (“ASEX”; McGahuey et al., J Sex Marital Ther. 26:25-40 (2000)), the entire disclosures of which are incorporated herein by reference. For assessment using a questionnaire, a measure of sexual dysfunction function is increased when the score in the appropriate domain, subscale or subtest is indicative of sexual dysfunction, as established for that questionnaire. For instance, a female's sexual interest is considered reduced, when assessed using the CSFQ, if the subscale for sexual interest score is less than or equal to 9. Conversely, sexual dysfunction is considered improved when the score in the appropriate domain, subscale or subtest is indicative of higher (e.g., normal or desired) sexual function. For a clinician's assessment, sexual dysfunction may be assessed in comparison to a previous point in time for the patient and/or in comparison to a patient's peers with respect to age, gender, sexual experience, and health, or may also be determined via a validated questionnaire administered by the clinician.

According to embodiments, the efficacy and safety of the pharmaceutical compositions described herein in the treatment of the symptoms of VVA may be determined. According to embodiments, the size, effect, cytology, histology, and variability of the VVA may be determined using various endpoints to determine efficacy and safety of the pharmaceutical compositions described herein or as otherwise accepted in the art, at present or as further developed. One source of endpoints is with the US Food and Drug Administration's (FDA) published guidelines for treatment of VVA with estradiol.

According to embodiments, a method of treating VVA, including dyspareunia, vaginal dryness, and estrogen-deficient urinary states (including urinary tract infections), is provided that allows a subject to be ambulatory immediately or within minutes after a gelatin capsule containing the pharmaceutical compositions disclosed herein are administered. According to embodiments, a gelatin capsule containing a pharmaceutical composition as disclosed herein is administered by digitally inserting the gelatin capsule containing the pharmaceutical composition into the vagina approximately two inches or inserting into the third of the vagina closest to the vaginal opening as shown in FIGS. 26A, 26B, and 26C. According to embodiments, the gelatin capsule adheres to the vaginal tissue and dissolves, ruptures, or otherwise disintegrates soon after being inserted into the vagina thereby releasing the pharmaceutical composition. The pharmaceutical composition spreads onto the vaginal tissue and is rapidly absorbed. According to embodiments, the gelatin capsule is also fully absorbed by the vaginal tissue. According to some embodiments, a viscosity enhancer such as TEFOSE 63 provides increased viscosity to ensure the pharmaceutical composition stays within the desired absorption area, thereby estrogenizing the vagina, labia, and/or vulva. The combination of high viscosity, bioadhesion, and rapid absorption prevents the need for subjects to remain supine after administration to allow the tissue to absorb the estradiol, thereby allowing subjects to be ambulatory immediately or almost immediately after administration.

According to embodiments, a method for treating VVA, including dyspareunia, vaginal dryness, and estrogen-deficient urinary states (including urinary tract infections), without causing non-natural discharge (e.g., discharge of a pharmaceutical composition or a component thereof) is provided. According to the method, a soft gelatin capsule is administered containing a liquid pharmaceutical composition that is able to be fully absorbed by the vaginal tissue. According to embodiments, the pharmaceutical composition itself is fully absorbed by the vaginal tissue. According to embodiments, the pharmaceutical composition and gelatin capsule are administered in a volume and size, respectively, that allows a subject's vaginal tissue to fully absorb the pharmaceutical composition. According to embodiments, such absorption will occur contemporaneously with the subject being ambulatory. According to the method, the gelatin capsule and liquid pharmaceutical composition are fully absorbed by the vaginal tissue, wherein the only discharge that occurs after estrogenizing the vagina is natural discharge that a woman would have experienced prior to menopause. “Natural” vaginal discharge refers to a small amount of fluid that flows out of the vagina each day, carrying out old cells that have lined the vagina. Natural discharge is usually clear or milky. Non-natural discharge can refer to discharge that is higher in volume than natural discharge, different in color than natural discharge, or different in consistency than natural discharge. Non-natural discharge can also refer to the discharge (e.g., leaking) of a pharmaceutical composition from the vagina.

According to embodiments, a method of treating VVA, including dyspareunia, vaginal dryness, and estrogen-deficient urinary states (including urinary tract infections), using a liquid pharmaceutical composition is provided. According to the method, a soft gelatin capsule containing a liquid composition for treating VVA is provided to a subject. The subject inserts the soft gelatin capsule containing the liquid composition for treating VVA into their vagina either digitally or with an applicator, wherein the soft gelatin capsule dissolves, ruptures, or disintegrates and the liquid composition is released into the vagina. According to embodiments, the liquid composition for treating VVA is a pharmaceutical composition disclosed herein. According to embodiments, the subject inserts the gelatin capsule about two inches into the vagina, or in the third of the vagina closest to the vaginal opening. According to embodiments, the subject is ambulatory immediately after or soon after administration.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within two weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within four weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the four week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within eight weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the eight week point of measurement, the estradiol is not detected systemically when measured using standard pharmaceutical pharmacokinetic parameters, such as AUC and Cₘₐₓ.

According to embodiments, a method is disclosed herein for treating VVA, including dyspareunia, vaginal dryness, and estrogen-deficient urinary states (including urinary tract infections), comprising improving the symptoms of VVA, compared to placebo or baseline, within ten weeks by vaginally administering a composition for the treatment of VVA. One of skill in the art will understand that the improvements can be assessed statistically as described herein, and that any improvement can be a statistically significant improvement. According to embodiments, the composition for the treatment of VVA is a liquid pharmaceutical composition as disclosed herein. According to embodiments, the composition for the treatment of VVA is a liquid containing from 1 μg to 25 μg of estradiol. According to embodiments, the method of administration is a method disclosed herein, including the insertion method shown in FIGS. 26A, 26B, and 26C. According to embodiments, at the two week point of measurement and/or the ten week point of measurement, the estradiol is not detected systemically when measured using standard pharmaceutical pharmacokinetic parameters, such as AUC and Cₘₐₓ.

According to embodiments, a method for treating VVA, including dyspareunia, vaginal dryness, and estrogen-deficient urinary states (including urinary tract infections), comprising administering a composition containing estradiol for the treatment of VVA is provided, wherein the method improves the symptoms of VVA, compared with baseline or placebo, in at least one of two weeks, four weeks, six weeks, eight weeks, or twelve weeks, wherein the estradiol is not detected systemically using standard pharmaceutical pharmacokinetic parameters, such as AUC and Cₘₐₓ. One of skill in the art will understand that the improvements can be assessed statistically as described herein, and that any improvement can be a statistically significant improvement. According to embodiments, the composition containing estradiol is a liquid composition as disclosed herein. According to embodiments, the composition contains 1 μg to 25 μg of estradiol.

According to embodiments, a method for reestrogenizing the vagina, labia, or vulva is provided, wherein the method comprises administering a composition containing estradiol for the treatment of VVA, wherein the composition is a liquid containing estradiol or a synthetic estrogen, and wherein the liquid spreads over a surface area of the vagina, labia, or vulva which is larger than the area covered by a solid composition. For example, the liquid can spread over a surface area ranging from about 50 cm² to about 120 cm² (e.g., from about 50 cm² to about 60 cm²; or from about 60 cm² to about 70 cm²; or from about 70 cm² to about 80 cm²; or from about 80 cm² to about 90 cm²; or from about 90 cm² to about 100 cm²; or from about 100 cm² to about 110 cm²; or from about 110 cm² to about 120 cm²; or from about 65 cm² to about 110 cm²). According to embodiments, the subject inserts a liquid composition into her vagina in a capsule, such as a hard or soft gelatin capsule, that then dissolves, ruptures, disintegrates, or otherwise releases the liquid in the vagina. According to embodiments, the liquid contains at least one of a bio-adhesive or viscosity enhancer to prevent the liquid from discharging from the vagina before the estradiol or synthetic estrogen can be absorbed into the vaginal tissue in a dose sufficient to effect reestrongenization of the vagina. According to embodiments, the vagina will be statistically significantly reestrogenized within two weeks of administration compared to baseline or placebo levels. According to embodiments, the vagina will be statistically significantly reestrogenized within four weeks of administration compared to baseline or placebo levels.

According to embodiments, the vagina will be statistically significantly reestrogenized within six weeks of administration compared to baseline or placebo levels. According to embodiments, the vagina will be statistically significantly reestrogenized within eight weeks of administration compared to baseline or placebo levels. According to embodiments, the vagina will be statistically significantly reestrogenized within ten weeks of administration compared to baseline or placebo levels. According to embodiments, the vagina will be statistically significantly reestrogenized within twelve or more weeks of administration compared to baseline or placebo levels.

### VII. MEASUREMENT OF EFFICACY

According to embodiments, administration of the pharmaceutical compositions described herein resulted in treatment of the VVA, as well as improvement of one or more of the associated symptoms. Patients with VVA experience shrinking of the vaginal canal in both length and diameter and the vaginal canal has fewer glycogen-rich vaginal cells to maintain moisture and suppleness. In addition, the vaginal wall can become thin, pale, dry, or sometimes inflamed (atrophic vaginitis). These changes can manifest as a variety of symptoms collectively referred to as VVA. Such symptoms include, without limitations, an increase in vaginal pH; reduction of vaginal epithelial integrity, vaginal secretions, or epithelial surface thickness; pruritus; vaginal dryness; dyspareunia (pain or bleeding during sexual intercourse); urinary tract infections; or a change in vaginal color.

It will be recognized by a person of ordinary skill in the art that efficacy of treating VVA can be measured according to any of a number of parameters. According to embodiments, efficacy is measured as an improvement in one or more clinical parameters, wherein the parameters are selected from the group consisting of: (i) an increase in the percentage of vaginal superficial cells, (ii) a decrease in the percentage of vaginal parabasal cells, (iii) a decrease in vaginal pH, (iv) a decrease in the severity of moderate to severe dyspareunia, (v) a decrease in vaginal dryness, (vi) a decrease in vulvar or vaginal itching or irritation, (vii) an increase of vaginal mucosa, (viii) an assessment of standard pK parameters, (ix) a change in the Female Sexual Function Index (FSFI), (x) visual vaginal assessment, and (xi) the subject's self-reported response (e.g., a subject's self-reported symptoms or self-reported change in one or more symptoms). In some embodiments, the subject's clinical response is measured as an improvement in one or more clinical parameters selected from the group consisting of an increase in the percentage of vaginal superficial cells, a decrease in the percentage of vaginal parabasal cells, a decrease in vaginal pH, and a decrease in the severity of moderate to severe dyspareunia. In some embodiments, efficacy of treatment is measured as a decrease in the severity of moderate to severe dyspareunia. In some embodiments, efficacy of treatment is measured by visual assessment or by a subject's self-reported response. In some embodiments, efficacy of treatment is measured by a subject's self-reported response, such as the subject's reported symptoms or changes in symptoms of vulvar or vaginal atrophy including vaginal pain associated with sexual activity, vaginal dryness, vulvar or vaginal itching or irritation.

According to embodiments, efficacy can be measured at one or more timepoints following the first administration of the estradiol composition (e.g., softgel capsule), e.g., at about one, two, three, four, five, six, seven, eight, nine, ten weeks or longer after the first administration of the estradiol composition. In some embodiments, efficacy can be measured periodically at multiple timepoints, e.g., about every week, about every two weeks, about every three weeks, about every four weeks, about every five weeks, about every six weeks, about every seven weeks, about every eight weeks, about every nine weeks, or about every ten weeks.

According to embodiments, efficacy is measured as a reduction of vulvar and vaginal atrophy in a patient back to premenopausal conditions. According to embodiments, the change is measured as a reduction in the severity of one or more atrophic effects measured at baseline (screening, Day 1) and compared to a measurement taken at Day 15 (end of treatment). Severity of the atrophic effect may be measured using a scale of 0 to 3 where, for example, none=0, mild=1, moderate=2, or severe=3. Such scoring is implemented to evaluate the pre-treatment condition of patients; to determine the appropriate course of a treatment regime; such as dosage, dosing frequency, and duration, among others; and post-treatment outcomes.

One of the symptoms of VVA is increased vaginal pH. In further aspects of this disclosure, treatment with the pharmaceutical compositions described herein resulted in a decrease in vaginal pH. A decrease in vaginal pH is measured as a decrease from the vaginal pH at baseline (screening) to the vaginal pH at Day 15, according to embodiments. In some embodiments, a pH of 5 or greater may be associated with VVA. In some embodiments, pH is measured using a pH indicator strip placed against the vaginal wall. In some embodiments, a change in vaginal pH is a change in a patient's vaginal pH to a pH of less than about pH 5.0. In some embodiments, a subject's vaginal pH may be less than about pH 4.9, pH 4.8, pH 4.7, pH 4.6, pH 4.5, pH 4.4, pH 4.3, pH 4.2, pH 4.1, pH 4.0, pH 3.9, pH 3.8, pH 3.7, pH 3.6, or pH 3.5.

According to embodiments, treatment with the pharmaceutical compositions described herein resulted in improvements in the vaginal Maturation Index. The Maturation Index is measured as a change in cell composition. According to embodiments and as related to VVA, a change in cell composition is measured as the change in percent of composition or amount of parabasal vaginal cells, intermediate cells, and superficial vaginal cells, such as a change in the composition or amount of parabasal vaginal cells compared with or, relative to, a change in superficial vaginal cells. A subject having VVA symptoms often has an increased number of parabasal cells and a reduced number of superficial cells (e.g., less than about 5%) compared with women who do not suffer from VVA. Conversely, a subject having decreasing VVA symptoms, or as otherwise responding to treatment, may demonstrate an improvement in the Maturation Index, specifically a decrease in the amount of parabasal cells or an increase in the amount of superficial cells compared to baseline (screening). In embodiments, a decrease in parabasal cells is measured as a reduction in the percent of parabasal cells; the percent reduction may be at least about an 85%, 80%, 75%, 70%, 65%, 60%, 55%, 50%, 45%, 40%, 35%, 30%, 25%, 20%, 15% or 10% reduction in the number of parabasal cells. In embodiments, a percent reduction may be at least about a 54% reduction in the number of parabasal cells. In embodiments, an increase in superficial cells is measured as an increase in the percent of superficial cells; the percent increase in superficial cells may be at least about 5%, 10%, 15%, 20%, 25%, 30%, 35%, 40%, 45%, or 50% increase in the number of superficial cells. In further embodiments, a percent increase may be at least about a 35% increase in the number of superficial cells.

In some embodiments, an improvement in the Maturation Index is assessed as a change over time. For example, as a change in cell composition measured at a baseline (screening) at Day 1 compared to the cell composition measured at Day 15. The change in cell composition may also be assessed as a change in the amount of parabasal cells over time, optionally in addition to measuring changes in parabasal cells and superficial cells as described above. Such cells may be obtained from the vaginal mucosal epithelium through routine gynecological examination and examined by means of a vaginal smear.

In various further aspects of this disclosure, treatment with the pharmaceutical compositions described herein resulted in any of: an increase in superficial cells; a decrease in parabasal cells; and an increase in intermediate cells.

In further aspects of this disclosure, samples may be collected to determine hormone levels, in particular, estradiol levels. In some embodiments, blood samples may be taken from a subject and the level of estradiol measured (pg/mL). In some embodiments, estradiol levels may be measured at 0 hours (for example, at time of first treatment), at 1 hour (for example, post first treatment), at 3 hours, and at 6 hours. In some embodiments, samples may be taken at day 8 (for example, post first treatment) and at day 15 (for example, one day post the last treatment on day 14). In some embodiments, descriptive statistics of plasma estradiol concentrations at each sampling time and observed Cₘₐₓ and Tₘₐₓ values may be measured and the AUC calculated.

In some embodiments, a suppository can comprise about 25 μg of estradiol. In such cases, administration of the suppository to a patient can provide, in a plasma sample from the patient, parameters including one or more parameters selected from: 1) a corrected geometric mean peak plasma concentration (Cₘₐₓ) of estradiol of about 19 pg*hr/mL to about 29 pg*hr/mL (e.g., 19.55 pg*hr/mL to about 28.75 pg*hr/mL); or 2) a corrected geometric mean area under the curve (AUC)₀₋₂₄ of estradiol of about 75 pg*hr/mL to about 112 pg*hr/mL (e.g., 75.82 pg*hr/mL to about 111.50). In some embodiments, administration of the suppository to a patient provides, in a plasma sample from the patient, one or more parameters selected from: 1) a corrected geometric mean peak plasma concentration (Cₘₐₓ) of estrone of about 9 pg*hr/mL to about 14 pg*hr/mL (e.g., 9.17 pg*hr/mL to about 13.49 pg*hr/mL); and 2) a corrected geometric mean area under the curve (AUC)₀₋₂₄ of estrone of about 43 pg*hr/mL to about 65 pg*hr/mL (e.g., 43.56 pg*hr/mL to about 64.06 pg*hr/mL). In some embodiments, administration of the suppository to a patient provides, in a plasma sample from the patient, provides one or more parameters selected from: 1) a corrected geometric mean peak plasma concentration (Cₘₐₓ) of estrone sulfate of about 416 pg*hr/mL to about 613 pg*hr/mL (e.g., 416.53 pg*hr/mL to about 612.55 pg*hr/mL); and 2) a corrected geometric mean area under the curve (AUC)₀₋₂₄ of estrone sulfate of about 3598 pg*hr/mL to about 5291 pg*hr/mL (e.g., 3598.04 pg*hr/mL to about 5291.24 pg*hr/mL).

In some embodiments, a suppository includes about 25 μg of estradiol. In some such embodiments, administration of the suppository to a patient can provide, in a plasma sample from the patient, parameters including one or more parameters selected from: 1) a corrected geometric mean peak plasma concentration (Cₘₐₓ) of estradiol ranging from about 20.9 pg/mL to about 32.8 pg/mL (e.g., 20.96 pg/mL to about 32.75 pg/mL); 2) a corrected geometric mean area under the curve (AUC)₀₋₂₄ of estradiol ranging from about 104.3 pg*hr/mL to about 163.1 pg*hr/mL (e.g., 104.32 pg*hr/mL to about 163.0 pg*hr/mL); and 3) an average concentration (C_{avg}) of estradiol ranging from about 4.3 pg/mL to about 6.8 pg/mL (e.g., 4.32 pg/mL to about 6.75 pg/mL), as assessed at day 1.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol of about 26.2 pg/mL; 2) a corrected geometric mean area under the curve (AUC)₀₋₂₄ of estradiol of about 130 pg*hr/mL; and 3) an average concentration (C_{avg}) of estradiol of about 5.4 pg/mL, as assessed at day 1.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol ranging from about 9.5 pg/mL to about 15.1 pg/mL (e.g., 9.60 pg*hr/mL to about 15.00 pg/mL); 2) a corrected geometric mean area under the curve (AUC)₀₋₂₄ of estradiol ranging from about 67.6 pg*hr/mL to about 105.8 pg*hr/mL (e.g., 67.68 pg*hr/mL to about 105.75 pg*hr/mL); and 3) an average concentration (C_{avg}) of estradiol ranging from about 2.7 pg/mL to about 4.4 pg/mL (e.g., 2.80 pg/mL to about 4.38 pg/mL) of estradiol as assessed at day 14.

In some embodiments, administration of a suppository comprising about 25 μg of estradiol to a patient can provide, in a plasma sample from the patient, parameters including one or more parameters selected from: 1) a corrected geometric mean peak plasma concentration (Cₘₐₓ) of estradiol of about 12.0 pg/mL; 2) a corrected geometric mean area under the curve (AUC)₀₋₂₄ of estradiol of about 84.6 pg*hr/mL; and 3) an average concentration (C_{avg}) of estradiol of about 3.5 pg/mL, as assessed at day 14.

In some embodiments, administration of a suppository comprising about 25 μg of estradiol to a patient provides, in a plasma sample from the patient, one or more parameters selected from: 1) a corrected geometric mean peak plasma concentration (Cₘₐₓ) of estrone conjugates ranging from about 158.8 pg/mL to about 248.3 pg/mL (e.g., 158.88 hr/mL to about 248.25 pg*hr/mL); and 2) a corrected geometric mean area under the curve (AUC)₀₋₂₄ of estrone conjugates ranging from about 1963.1 pg*hr/mL to about 3067.6 pg*hr/mL (e.g., 1963.20 pg*hr/mL to about 3067.50 pg*hr/mL) as assessed at day 1.

In some embodiments, administration of a suppository comprising about 25 μg of estradiol to a patient provides, in a plasma sample from the patient, one or more parameters selected from: 1) a corrected geometric mean peak plasma concentration (Cₘₐₓ) of estrone conjugates of about 198.6 pg/mL; and 2) a corrected geometric mean area under the curve (AUC)₀₋₂₄ of estrone conjugates of about 2454 pg*hr/mL as assessed at day 1.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estradiol ranging from about 173.5 pg*hr/mL to about 271.3 pg*hr/mL (e.g., from 173.60 pg*hr/mL to about 271.25 pg*hr/mL; or about 217 pg*hr/mL), as assessed at day 1; 2) a corrected arithmetic mean peak plasma concentration (C_{avg[0-24]}) of estradiol ranging from about 7.2 pg/mL to about 11.4 pg/mL (e.g., from 7.25 pg/mL to about 11.33 pg/mL; or about 9.06 pg/mL), as assessed at day 1; 3) an unadjusted arithmetic mean area under the curve (AUC)₀₋₂₄ of estradiol ranging from about 137.5 pg*hr/mL to about 215.1 pg*hr/mL (e.g., from 137.60 pg*hr/mL to about 215.00 pg*hr/mL; or about 172 pg*hr/mL), as assessed at day 14; and 4) a corrected arithmetic mean peak plasma concentration (C_{avg[0-24]}) of estradiol ranging from about 5.7 pg/mL to about 9.0 pg/mL (e.g., from 5.72 pg/mL to about 8.94 pg/mL; or about 7.15 pg/mL), as assessed at day 14.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estrone ranging from about 335.1 pg*hr/mL to about 523.8 pg*hr/mL (e.g., from 335.20 pg*hr/mL to about 523.75 pg*hr/mL; or about 419 pg*hr/mL), as assessed at day 1; 2) a corrected arithmetic mean peak plasma concentration (C_{avg[0-24]}) of estrone ranging from about 13.9 pg/mL to about 21.9 pg/mL (e.g., from 14.00 pg/mL to about 21.88 pg/mL; or about 17.5 pg/mL), as assessed at day 1; 3) an unadjusted arithmetic mean area under the curve (AUC)₀₋₂₄ of estrone ranging from about 343.1 pg*hr/mL to about 536.2 pg*hr/mL (e.g., from 343.20 pg*hr/mL to about 536.25 pg*hr/mL; or about 429 pg*hr/mL), as assessed at day 14; and 4) a corrected arithmetic mean peak plasma concentration (C_{avg[0-24]}) of estrone ranging from about 14.3 pg/mL to about 22.4 pg/mL (e.g., from 14.32 pg/mL to about 22.38 pg/mL; or about 17.9 pg/mL), as assessed at day 14.

In some embodiments, administration of a suppository comprising about 25 μg of estradiol to a patient provides, in a plasma sample from the patient, one or more parameters selected from: 1) an unadjusted arithmetic mean area under the curve (AUC)₀₋₂₄ of estrone conjugates ranging from about 7,300.7 pg*hr/mL to about 11,407.6 pg*hr/mL (e.g., from 7,300.80 pg*hr/mL to about 11,407.50 pg*hr/mL; or about 9,126 pg*hr/mL), as assessed at day 1; 2) a corrected arithmetic mean peak plasma concentration (C_{avg[0-24]}) of estrone conjugates ranging from about 303.9 pg/mL to about 475.1 pg/mL (e.g., from 304.00 pg/mL to about 475.00 pg/mL; or about 380 pg/mL), as assessed at day 1; 3) an unadjusted arithmetic mean area under the curve (AUC)₀₋₂₄ of estrone conjugates ranging from about 7,943.9 pg*hr/mL to about 12,412.6 pg*hr/mL (e.g., from 7,944.00 pg*hr/mL to about 12,412.50 pg*hr/mL; or about 9,930 pg*hr/mL), as assessed at day 14; and 4) a corrected arithmetic mean peak plasma concentration (C_{avg[0-24]}) of estrone conjugates ranging from about 331.1 pg/mL to about 517.4 pg/mL (e.g., from 331.20 pg/mL to about 517.50 pg/mL; or about 414 pg/mL), as assessed at day 14.

In some embodiments, a suppository can comprise about 10 μg of estradiol. In such cases, administration of the suppository to a patient can provide, in a plasma sample from the patient, one or more parameters selected from: 1) a corrected geometric mean peak plasma concentration (Cₘₐₓ) of estradiol of about 12 pg*hr/mL to about 18 pg*hr/mL (e.g., 12.22 pg*hr/mL to about 17.98 pg*hr/mL); 2) a corrected geometric mean area under the curve (AUC)₀₋₂₄ of estradiol of about 42 pg*hr/mL to about 63 pg*hr/mL (e.g., 42.18 pg*hr/mL to about 62.02 pg*hr/mL); and 3) a corrected geometric mean time to peak plasma concentration (Tₘₐₓ) of estradiol of about 1 hrs to about 3 hrs (e.g., 1.49 hrs to about 2.19 hrs). In some embodiments, administration of the suppository to a patient provides, in a plasma sample from the patient, one or more parameters selected from: 1) a corrected geometric mean peak plasma concentration (Cₘₐₓ) of estrone of about 4 pg*hr/mL to about 7 pg*hr/mL (e.g., 4.38 pg*hr/mL to about 6.44 pg*hr/mL); 2) a corrected geometric mean area under the curve (AUC)₀₋₂₄ of estrone of about 20 pg*hr/mL to about 31 pg*hr/mL (e.g., 20.60 pg*hr/mL to about 30.30 pg*hr/mL); and 3) a corrected geometric mean time to peak plasma concentration (Tₘₐₓ) of estrone of about 4 hrs to about 8 hrs (e.g., 4.99 hrs to about 7.34 hrs). In some embodiments, administration of the suppository to a patient provides, in a plasma sample from the patient, one or more parameters selected from: 1) a corrected geometric mean peak plasma concentration (Cₘₐₓ) of estrone sulfate of about 10 pg*hr/mL to about 16 pg*hr/mL (e.g., 10.34 pg*hr/mL to about 15.20 pg*hr/mL); 2) a corrected geometric mean area under the curve (AUC)₀₋₂₄ of estrone sulfate of about 56 pg*hr/mL to about 84 pg*hr/mL (e.g., 56.61 pg*hr/mL to about 83.25 pg*hr/mL); and 3) a corrected geometric mean time to peak plasma concentration (Tₘₐₓ) of estrone sulfate of about 4 hrs to about 7 hrs (e.g., 4.67 hrs to about 6.86 hrs).

In some embodiments, a suppository includes about 10 μg of estradiol. In some such embodiments, administration of the suppository to a patient can provide, in a plasma sample from the patient, a corrected geometric mean peak plasma concentration (Cₘₐₓ) of estradiol ranging from about 4.7 pg/mL to about 7.6 pg/mL (e.g., 4.80 pg*hr/mL to about 7.50 pg*hr/mL), as assessed at day 1. In some embodiments, administration of a suppository comprising about 10 μg of estradiol to a patient can provide, in a plasma sample from the patient, a corrected geometric mean peak plasma concentration (Cₘₐₓ) of estradiol ranging from about 2.3 pg*hr/mL to about 3.8 pg*hr/mL (e.g., 2.40 pg*hr/mL to about 3.75 pg*hr/mL) of estradiol as assessed at day 14.

In some embodiments, administration of a suppository comprising about 10 μg of estradiol to a patient provides, in a plasma sample from the patient, a corrected geometric mean peak plasma concentration (Cₘₐₓ) of estradiol of about 6.0 pg/mL, as assessed at day 1. In some embodiments, administration of a suppository comprising about 10 μg of estradiol to a patient can provide, in a plasma sample from the patient, a corrected geometric mean peak plasma concentration (Cₘₐₓ) of estradiol of about 3.0 pg/mL, as assessed at day 14.

In some embodiments, administration of a suppository comprising about 10 μg of estradiol to a patient provides, in a plasma sample from the patient, a corrected geometric mean area under the curve (AUC)₀₋₂₄ of estradiol ranging from about 17.5 pg/mL to about 27.4 pg/mL (e.g., 17.52 pg*hr/mL to about 27.37 pg*hr/mL), as assessed at day 1. In some embodiments, administration of a suppository comprising about 10 μg of estradiol to a patient can provide, in a plasma sample from the patient, a corrected geometric mean area under the curve (AUC)₀₋₂₄ of estradiol ranging from about 10.9 pg*hr/mL to about 17.2 pg*hr/mL (e.g., 10.96 pg*hr/mL to about 17.13 pg*hr/mL) of estradiol as assessed at day 14.

In some embodiments, administration of a suppository comprising about 10 μg of estradiol to a patient provides, in a plasma sample from the patient, a corrected geometric mean area under the curve (AUC)₀₋₂₄ of estradiol of about 21.9 pg*hr/mL, as assessed at day 1. In some embodiments, administration of a suppository comprising about 10 μg of estradiol to a patient can provide, in a plasma sample from the patient, a corrected geometric mean area under the curve (AUC)₀₋₂₄ of estradiol of about 13.7 pg*hr/mL, as assessed at day 14.

In some embodiments, administration of a suppository comprising about 10 μg of estradiol to a patient provides, in a plasma sample from the patient, an average concentration (C_{avg}) of estradiol ranging from about 0.6 pg/mL to about 1.1 pg/mL (e.g., 0.64 pg/mL to about 1.0 pg/mL), as assessed at day 1. In some embodiments, administration of a suppository comprising about 10 μg of estradiol to a patient can provide, in a plasma sample from the patient, an average concentration (C_{avg}) of estradiol ranging from about 0.1 pg/mL to about 0.3 pg/mL (e.g., 0.16 pg/mL to about 0.25 pg/mL) of estradiol as assessed at day 14.

In some embodiments, administration of a suppository comprising about 10 μg of estradiol to a patient provides, in a plasma sample from the patient, an average concentration (C_{avg}) of estradiol of about 0.8 pg/mL, as assessed at day 1. In some embodiments, administration of a suppository comprising about 10 μg of estradiol to a patient can provide, in a plasma sample from the patient, an average concentration (C_{avg}) of estradiol of about 0.2 pg/mL, as assessed at day 14.

In some embodiments, administration of a suppository comprising about 10 μg of estradiol to a patient provides, in a plasma sample from the patient, one or more parameters selected from: 1) a corrected geometric mean peak plasma concentration (Cₘₐₓ) of estrone conjugates ranging from about 72.1 pg/mL to about 112.8 pg/mL (e.g., 72.16 pg/mL to about 112.75 pg/mL); and 2) an average concentration (C_{avg}) of estrone conjugates ranging from about 6.3 pg/mL to about 10.1 pg/mL (e.g., 6.40 pg/mL to about 10.00 pg/mL) as assessed at day 1.

In some embodiments, administration of a suppository comprising about 10 μg of estradiol to a patient provides, in a plasma sample from the patient, one or more parameters selected from: 1) a corrected geometric mean peak plasma concentration (Cₘₐₓ) of estrone conjugates of about 90.2 pg/mL; and 2) an average concentration (C_{avg}) of estrone conjugates of about 8.0 pg/mL, as assessed at day 1.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estradiol ranging from about 110.3 pg*hr/mL to about 172.6 pg*hr/mL (e.g., from 110.40 pg*hr/mL to about 172.50 pg*hr/mL; or about 138 pg*hr/mL), as assessed at day 1; 2) a corrected arithmetic mean peak plasma concentration (C_{avg[0-24]}) of estradiol ranging from about 4.6 pg/mL to about 7.8 pg/mL (e.g., from 4.61 pg/mL to about 7.20 pg/mL; or about 5.76 pg/mL), as assessed at day 1; 3) an unadjusted arithmetic mean area under the curve (AUC)₀₋₂₄ of estradiol ranging from about 87.9 pg*hr/mL to about 137.4 pg*hr/mL (e.g., from 88.00 pg*hr/mL to about 137.50 pg*hr/mL; or about 110 pg*hr/mL), as assessed at day 14; and 4) a corrected arithmetic mean peak plasma concentration (C_{avg[0-24]}) of estradiol ranging from about 3.6 pg/mL to about 5.8 pg/mL (e.g., from 3.67 pg/mL to about 5.74 pg/mL; or about 4.59 pg/mL), as assessed at day 14.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estrone ranging from about 370.3 pg*hr/mL to about 578.8 pg*hr/mL (e.g., from 370.40 pg*hr/mL to about 578.75 pg*hr/mL; or about 463 pg*hr/mL), as assessed at day 1; 2) a corrected arithmetic mean peak plasma concentration (C_{avg[0-24]}) of estrone ranging from about 15.4 pg/mL to about 24.2 pg/mL (e.g., from 15.44 pg/mL to about 24.13 pg/mL; or about 19.3 pg/mL), as assessed at day 1; 3) an unadjusted arithmetic mean area under the curve (AUC)₀₋₂₄ of estrone ranging from about 371.1 pg*hr/mL to about 580.1 pg*hr/mL (e.g., from 371.20 pg*hr/mL to about 580.00 pg*hr/mL; or about 464 pg*hr/mL), as assessed at day 14; and 4) a corrected arithmetic mean peak plasma concentration (C_{avg[0-24]}) of estrone ranging from about 15.4 pg/mL to about 24.2 pg/mL (e.g., from 15.44 pg/mL to about 24.13 pg/mL; or about 19.3 pg/mL), as assessed at day 14.

In some embodiments, administration of a suppository comprising about 10 μg of estradiol to a patient provides, in a plasma sample from the patient, one or more parameters selected from: 1) an unadjusted arithmetic mean area under the curve (AUC)₀₋₂₄ of estrone conjugates ranging from about 4,745.5 pg*hr/mL to about 7,414.9 pg*hr/mL (e.g., from 4,745.60 pg*hr/mL to about 7,415.00 pg*hr/mL; or about 5,932 pg*hr/mL), as assessed at day 1; 2) a corrected arithmetic mean peak plasma concentration (C_{avg[0-24]}) of estrone conjugates ranging from about 197.5 pg/mL to about 308.8 pg/mL (e.g., from 197.60 pg/mL to about 308.75 pg/mL; or about 247 pg/mL), as assessed at day 1; 3) an unadjusted arithmetic mean area under the curve (AUC)₀₋₂₄ of estrone conjugates ranging from about 7,182.3 pg*hr/mL to about 11,222.6 pg*hr/mL (e.g., from 7,182.40 pg*hr/mL to about 11,222.50 pg*hr/mL; or about 8,978 pg*hr/mL), as assessed at day 14; and 4) a corrected arithmetic mean peak plasma concentration (C_{avg[0-24]}) of estrone conjugates ranging from about 299.1 pg/mL to about 467.6 pg/mL (e.g., from 299.20 pg/mL to about 467.50 pg/mL; or about 374 pg/mL), as assessed at day 14.

In some embodiments, a suppository can comprise about 4 μg of estradiol. In such cases, administration of the suppository to a patient can provide, in a plasma sample from the patient, one or more parameters selected from: 1) a corrected geometric mean peak plasma concentration (Cₘₐₓ) of estradiol of about 4 pg*hr/mL to about 8 pg*hr/mL; 2) a corrected geometric mean area under the curve (AUC)₀₋₂₄ of estradiol of about 16 pg*hr/mL to about 26 pg*hr/mL; and 3) a corrected geometric mean time to peak plasma concentration (Tₘₐₓ) of estradiol of about 0.25 hrs to about 2 hrs. In some embodiments, administration of the suppository to a patient provides, in a plasma sample from the patient, one or more parameters selected from: 1) a corrected geometric mean peak plasma concentration (Cₘₐₓ) of estrone of about 1 pg*hr/mL to about 3 pg*hr/mL; 2) a corrected geometric mean area under the curve (AUC)₀₋₂₄ of estrone of about 8 pg*hr/mL to about 13 pg*hr/mL; and 3) a corrected geometric mean time to peak plasma concentration (Tₘₐₓ) of estrone of about 1 hrs to about 4 hrs. In some embodiments, administration of the suppository to a patient provides, in a plasma sample from the patient, one or more parameters selected from: 1) a corrected geometric mean peak plasma concentration (Cₘₐₓ) of estrone sulfate of about 4 pg*hr/mL to about 7 pg*hr/mL; 2) a corrected geometric mean area under the curve (AUC)₀₋₂₄ of estrone sulfate of about 22 pg*hr/mL to about 34 pg*hr/mL; and 3) a corrected geometric mean time to peak plasma concentration (Tₘₐₓ) of estrone sulfate of about 1 hrs to about 3 hrs.

In some embodiments, a suppository includes about 4 μg of estradiol. In some such embodiments, administration of the suppository to a patient can provide, in a plasma sample from the patient, one or more parameters selected from: 1) a corrected geometric mean peak plasma concentration (Cₘₐₓ) of estradiol ranging from about 2.0 pg/mL to about 3.3 pg/mL (e.g., 2.08 pg*hr/mL to about 3.25 pg*hr/mL); and 2) a corrected geometric mean area under the curve (AUC)₀₋₂₄ of estradiol ranging from about 9.5 pg*hr/mL to about 15.1 pg*hr/mL (e.g.; 9.60 pg*hr/mL to about 15.0 pg*hr/mL), as assessed at day 1. In some embodiments, administration of a suppository comprising about 4 μg of estradiol to a patient can provide, in a plasma sample from the patient, one or more parameters selected from: 1) a corrected geometric mean peak plasma concentration (Cₘₐₓ) of estradiol ranging from about 1.0 pg*hr/mL to about 1.7 pg*hr/mL (e.g., 1.04 pg*hr/mL to about 1.63 pg*hr/mL) of estradiol, and 2) a corrected geometric mean area under the curve (AUC)₀₋₂₄ of estradiol ranging from about 5.7 pg*hr/mL to about 9.1 pg*hr/mL (e.g., 5.76 pg*hr/mL to about 9.0 pg*hr/mL).

In some embodiments, administration of a suppository comprising about 4 μg of estradiol to a patient provides, in a plasma sample from the patient, one or more parameters selected from: 1) a corrected geometric mean peak plasma concentration (Cₘₐₓ) of estradiol of about 2.6 pg/mL; and 2) a corrected geometric mean area under the curve (AUC)₀₋₂₄ of estradiol of about 12 pg*hr/mL, as assessed at day 1. In some embodiments, administration of a suppository comprising about 10 μg of estradiol to a patient can provide, in a plasma sample from the patient, one or more parameters selected from: 1) a corrected geometric mean peak plasma concentration (Cₘₐₓ) of estradiol of about 1.3 pg/mL; 2) a corrected geometric mean area under the curve (AUC)₀₋₂₄ of estradiol of about 7.2 pg*hr/mL, as assessed at day 14.

In some embodiments, administration of a suppository comprising about 4 μg of estradiol to a patient provides, in a plasma sample from the patient, a corrected geometric mean peak plasma concentration (Cₘₐₓ) of estrone conjugates ranging from about 0.3 pg/mL to about 0.5 pg/mL (e.g., 0.32 pg/mL to about 0.5 pg/mL) as assessed at day 1.

In some embodiments, administration of a suppository comprising about 4 μg of estradiol to a patient provides, in a plasma sample from the patient, a corrected geometric mean peak plasma concentration (Cₘₐₓ) of estrone conjugates of about 0.4 pg/mL as assessed at day 1.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estradiol ranging from about 73.3 pg*hr/mL to about 114.7 pg*hr/mL (e.g., from 73.36 pg*hr/mL to about 114.63 pg*hr/mL; or about 91.7 pg*hr/mL), as assessed at day 1; 2) a corrected arithmetic mean peak plasma concentration (C_{avg[0-24]}) of estradiol ranging from about 3.1 pg/mL to about 4.8 pg/mL (e.g., from 3.14 pg/mL to about 4.90 pg/mL; or about 3.92 pg/mL), as assessed at day 1; 3) an unadjusted arithmetic mean area under the curve (AUC)₀₋₂₄ of estradiol ranging from about 69.7 pg*hr/mL to about 108.9 pg*hr/mL (e.g., from 69.76 pg*hr/mL to about 109.00 pg*hr/mL; or about 87.2 pg*hr/mL), as assessed at day 14; and 4) a corrected arithmetic mean peak plasma concentration (C_{avg[0-24]}) of estradiol ranging from about 2.8 pg/mL to about 4.6 pg/mL (e.g., from 2.90 pg/mL to about 4.54 pg/mL; or about 3.63 pg/mL), as assessed at day 14.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estrone ranging from about 231.9 pg*hr/mL to about 362.4 pg*hr/mL (e.g., from 232.00 pg*hr/mL to about 362.50 pg*hr/mL; or about 290 pg*hr/mL), as assessed at day 1; 2) a corrected arithmetic mean peak plasma concentration (C_{avg[0-24]}) of estrone ranging from about 10.3 pg/mL to about 16.3 pg/mL (e.g., from 10.40 pg/mL to about 16.25 pg/mL; or about 13 pg/mL), as assessed at day 1; 3) an unadjusted arithmetic mean area under the curve (AUC)₀₋₂₄ of estrone ranging from about 261.5 pg*hr/mL to about 408.8 pg*hr/mL (e.g., from 261.60 pg*hr/mL to about 408.75 pg*hr/mL; or about 327 pg*hr/mL), as assessed at day 14; and 4) a corrected arithmetic mean peak plasma concentration (C_{avg[0-24]}) of estrone ranging from about 10.8 pg/mL to about 17.1 pg/mL (e.g., from 10.88 pg/mL to about 17.00 pg/mL; or about 13.6 pg/mL), as assessed at day 14.

In some embodiments, administration of a suppository comprising about 4 μg of estradiol to a patient provides, in a plasma sample from the patient, one or more parameters selected from: 1) an unadjusted arithmetic mean area under the curve (AUC)₀₋₂₄ of estrone conjugates ranging from about 4,062.3 pg*hr/mL to about 6,347.6 pg*hr/mL (e.g., from 4,062.40 pg*hr/mL to about 6,347.50 pg*hr/mL; or about 5,078 pg*hr/mL), as assessed at day 1; 2) a corrected arithmetic mean peak plasma concentration (C_{avg[0-24]}) of estrone conjugates ranging from about 172.7 pg/mL to about 270.1 pg/mL (e.g., from 172.80 pg/mL to about 270.00 pg/mL; or about 216 pg/mL), as assessed at day 1; 3) an unadjusted arithmetic mean area under the curve (AUC)₀₋₂₄ of estrone conjugates ranging from about 4,138.3 pg*hr/mL to about 6,466.3 pg*hr/mL (e.g., from 4,138.40 pg*hr/mL to about 6,466.25 pg*hr/mL; or about 5173 pg*hr/mL), as assessed at day 14; and 4) a corrected arithmetic mean peak plasma concentration (C_{avg[0-24]}) of estrone conjugates ranging from about 172.7 pg/mL to about 270.1 pg/mL (e.g., from 172.80 pg/mL to about 270.00 pg/mL; or about 216 pg/mL), as assessed at day 14.

A pharmaceutical composition provided herein can result in substantially local delivery of estradiol. For example, plasma concentrations of estradiol, estrone, and estrone sulfate measured in the plasma of a patient following administration of a pharmaceutical composition as provided herein be statistically similar to those measured following administration of a placebo formulation (i.e., a similar formulation lacking the estradiol). Accordingly, in some embodiments, the plasma concentrations of estradiol, estrone, or estrone sulfate measured following administration of a pharmaceutical composition provided herein may be low compared to RLD formulations.

In some embodiments, a suppository can include about 1 μg to about 25 μg of estradiol. Upon administration the suppository to a patient, a plasma sample from the patient can provide a corrected geometric mean peak plasma concentration (Cₘₐₓ) of estradiol that is less than about 30 pg*hr/mL. For example, administration of the suppository to a patient provides a corrected geometric mean peak plasma concentration (Cₘₐₓ) of estradiol that is less than about 18 pg*hr/mL. In some embodiments, administration of the suppository to a patient provides a corrected geometric mean area under the curve (AUC)₀₋₂₄ of estradiol that is less than about 112 pg*hr/mL. For example, administration of the suppository to a patient provides a corrected geometric mean area under the curve (AUC)₀₋₂₄ of estradiol that is less than about 63 pg*hr/mL.

In some embodiments, administration of the suppository to a patient provides a corrected geometric mean peak plasma concentration (Cₘₐₓ) of estrone that is less than about 14 pg*hr/mL. For example, administration of the suppository to a patient provides a corrected geometric mean peak plasma concentration (Cₘₐₓ) of estrone that is less than about 7 pg*hr/mL. In some embodiments, administration of the suppository to a patient provides a corrected geometric mean area under the curve (AUC)₀₋₂₄ of estrone that is less than about 65 pg*hr/mL. For example, administration of the suppository to a patient provides a corrected geometric mean area under the curve (AUC)₀₋₂₄ of estrone that is less than about 31 pg*hr/mL.

In some embodiments, administration of the suppository to a patient provides a corrected geometric mean peak plasma concentration (Cₘₐₓ) of estrone sulfate that is less than about 613 pg*hr/mL. For example, administration of the suppository to a patient provides a corrected geometric mean peak plasma concentration (Cₘₐₓ) of estrone sulfate that is less than about 16 pg*hr/mL. In some embodiments, administration of the suppository to a patient provides a corrected geometric mean area under the curve (AUC)₀₋₂₄ of estrone sulfate that is less than about 5291 pg*hr/mL. For example, administration of the suppository to a patient provides a corrected geometric mean area under the curve (AUC)₀₋₂₄ of estrone sulfate that is less than about 84 pg*hr/mL.

In further aspects of this disclosure, capsule disintegration may be determined. In some embodiments, delivery vehicle disintegration or absorption (presence or absence of the delivery vehicle after administration) at day 1 of treatment (for example, at 6 hours post first treatment) and at day 15 (for example, one day post the last treatment on day 14).

The pharmaceutical compositions can be formulated as described herein to provide desirable pharmacokinetic parameters in a subject (e.g., a female subject) to whom the composition is administered. In some embodiments, a pharmaceutical composition as described herein produces desirable pharmacokinetic parameters for estradiol in the subject. In some embodiments, a pharmaceutical composition as described herein produces desirable pharmacokinetic parameters for one or more metabolites of estradiol in the subject, for example, estrone or total estrone.

Following the administration of a composition comprising estradiol to a subject, the concentration and metabolism of estradiol can be measured in a sample (e.g., a blood, serum, or plasma sample) from the subject. Estradiol is typically converted reversibly to estrone, and both estradiol and estrone can be converted to the metabolite estriol. In postmenopausal women, a significant proportion of circulating estrogens exist as sulfate conjugates, especially estrone sulfate. Thus, estrone can be measured with respect to “estrone” amounts (excluding conjugates such as estrone sulfate) and “total estrone” amounts (including both free, or unconjugated, estrone and conjugated estrone such as estrone sulfate).

The pharmaceutical compositions of this disclosure can be characterized for one or more pharmacokinetic parameters of estradiol or a metabolite thereof following administration of the composition to a subject or to a population of subjects. These pharmacokinetic parameters include AUC, Cₘₐₓ, C_{avg}, and Tₘₐₓ. AUC is a determination of the area under the curve (AUC) plotting the blood, serum, or plasma concentration of drug along the ordinate (Y-axis) against time along the abscissa (X-axis). AUCs are well understood, frequently used tools in the pharmaceutical arts and have been extensively described. Cₘₐₓ is well understood in the art as an abbreviation for the maximum drug concentration in blood, serum, or plasma of a subject. Tₘₐₓ is well understood in the art as an abbreviation for the time to maximum drug concentration in blood, serum, or plasma of a subject.

In some embodiments, one or more pharmacokinetic parameters, e.g., AUC, Cₘₐₓ, C_{avg}, or Tₘₐₓ, is measured for estradiol. In some embodiments, one or more pharmacokinetic parameters, e.g., AUC, Cₘₐₓ, C_{avg}, or Tₘₐₓ, is measured for estrone. In some embodiments, one or more pharmacokinetic parameters, e.g., AUC, Cₘₐₓ, C_{avg}, or Tₘₐₓ, is measured for total estrone. Any pharmacokinetic parameter can be a “corrected” parameter, wherein the parameter is determined as a change over a baseline level.

Any of a variety of methods can be used for measuring the levels of estradiol, estrone, or total estrone in a sample, including immunoassays, mass spectrometry (MS), high performance liquid chromatography (HPLC) with ultraviolet fluorescent detection, liquid chromatography in conjunction with mass spectrometry (LC-MS), tandem mass spectrometry (MS/MS), and liquid chromatography-tandem mass spectrometry (LC-MS/MS). In some embodiments, the levels of estradiol, estrone, or total estrone are measured using a validated LC-MS/MS method. Methods of measuring hormone levels are well described in the literature.

### Statistical Measurements

According to embodiments, pharmacokinetics of the pharmaceutical composition disclosed herein are measured using statistical analysis. According to embodiments, Analysis of Variance (“ANOVA”) or Analysis of CoVariance (“ANCOVA”) are used to evaluate differences between a patient receiving treatment with a pharmaceutical composition comprising an active pharmaceutical composition (for example, a pharmaceutical composition comprising estradiol) and a patient receiving treatment with a placebo (for example, the same pharmaceutical composition but without estradiol) or a reference drug. A person of ordinary skill in the art will understand how to perform statistical analysis of the data collected.

### VIII. EXAMPLES

The following examples are of pharmaceutical compositions, delivery vehicles, and combinations thereof. Methods of making are also disclosed. Data generated using the pharmaceutical compositions disclosed herein are also disclosed.

### Example 1: Pharmaceutical Composition

In embodiments, estradiol is procured and combined with one or more pharmaceutically acceptable solubilizing agents. The estradiol is purchased as a pharmaceutical grade ingredient, often as micronized estradiol, although other forms can also be used. In embodiments, the pharmaceutical composition includes estradiol in a dosage strength of from about 1 μg to about 50 μg. In embodiments, the pharmaceutical composition includes 10 μg of estradiol. In embodiments, the pharmaceutical composition includes 25 μg of estradiol.

In embodiments, the estradiol is combined with pharmaceutically acceptable solubilizing agents, and, optionally, other excipients, to form a pharmaceutical composition. In embodiments, the solubilizing agent is one or more of CAPMUL MCM, MIGLYOL 812, GELUCIRE 39/01, GELUCIRE 43/01, GELUCIRE 50/13, and TEFOSE 63.

GELUCIRE 39/01 and GELUCIRE 43/01 each have an HLB value of 1. GELUCIRE 50/13 has an HLB value of 13. TEFOSE 63 has an HLB value of between 9 and 10.

Various combinations of pharmaceutically acceptable solubilizing agents were combined with estradiol and examined as shown in Table 1.

TABLE 1
Capmul MCM (“MCM”), Gelucire 39/01 (“39/01”), Gelucire 43/01 (“43/01”),
Gelucire 50/13 (“50/13”), and Tefose (“Tefose 63”)

| | | | Physical | Physical | | | |
|---|---|---|---|---|---|---|---|
| | | | state @ | state @ | | Melting | Dispersion |
| | | | Room | 37° C. after | Viscosity | Time @ | in water |
| # | Vehicle system | Ratio | Temperature | ~30 minutes | (cps) | 37° C. | 37° C. |
| 1 | MCM:39/01 | 8:2 | Solid | Clear liquid | 50 @ 37° C. | Start: 6 min | Small oil |
| | | | | | | Finish: 12 min | drops on top |
| 2 | MCM:39/01 | 7:3 | Solid | Clear liquid | | Start: 9 min | |
| | | | | | | Finish: 19 min | |
| 3 | MCM:39/01 | 6:4 | Solid | Clear liquid | | Start: 20 min | |
| | | | | | | Finish: 32 min | |
| 4 | MCM:43/01 | 8:2 | Solid | Liquid with | | | |
| | | | | solid particles | | | |
| 5 | MCM:43/01 | 7:3 | Solid | Liquid with | | | |
| | | | | solid particles | | | |
| 6 | MCM:50/13 | 9:1 | Liquid/ | Liquid/cloudy | 140 @ 25° C. | Clear after | Uniformly |
| | | | cloudy | | | 20 min | cloudy |
| | | | | | | | dispersion |
| 7 | MCM:50/13 | 8:2 | Liquid/ | Liquid/cloudy | 190 @ 25° C. | | Uniformly |
| | | | cloudy | | | | cloudy |
| | | | | | | | dispersion |
| 8 | MCM:50/13 | 7:3 | Semisolid | Semisolid | | | |
| 9 | MCM:TEFOSE | 9:1 | Semisolid | Liquid/cloudy | 150 @ 25° C. | Start: 1 min | Uniformly |
| | 63 | | | | | Finish: 5 min | cloudy |
| | | | | | | | dispersion |
| 10 | MCM:TEFOSE | 8:2 | Semisolid | Semisolid | 240 @ 25° C. | | Uniformly |
| | 63 | | | | | | cloudy |
| | | | | | | | dispersion |
| 11 | MCM:TEFOSE | 7:3 | Semisolid | Semisolid | 380 @ 25° C. | Semisolid | Uniformly |
| | 63 | | | | | after 30 min | cloudy |
| | | | | | | at 37° C. | dispersion |
| | | | | | | doesn't melt | |
| | | | | | | at 41° C. | |
| 12 | MIGLYOL 812: | 9:1 | Semisolid | Semisolid | 140 @ 25° C. | | 2 phases, oil |
| | 50/13 | | | | | | on top |
| 13 | MIGLYOL 812: | 9:1 | Liquid/ | Liquid/cloudy | 90 @ 25° C. | Start: 1 min | 2 phases, oil |
| | TEFOSE 63 | | cloudy | | | Finish: 5 min | on top |

Pharmaceutical compositions in Table 1 that were liquid or semisolid at room temperature were tested using a Brookfield viscometer (Brookfield Engineering Laboratories, Middleboro, MA) at room temperature. Pharmaceutical compositions appearing in Table 1 that were solid at ambient temperature were tested using a Brookfield viscometer at 37° C.

Pharmaceutical compositions appearing in Table 1 that were solid at room temperature were assessed at 37° C. to determine their melting characteristics. The viscosity of the gels can be important during encapsulation of the formulation. For example, in some cases, it is necessary to warm the formulation prior to filing of the gelatin capsules. In addition, the melting characteristics of the composition can have important implications following administration of the formulation into the body. For example, in some embodiments, the formulation will melt at temperatures below about 37° C. Pharmaceutical Composition 11 (Capmul MCM/Tefose 63), for example, did not melt at 37° C. or 41° C.

A dispersion assessment of the pharmaceutical compositions appearing in Table 1 was performed. The dispersion assessment was performed by transferring 300 mg of each vehicle system in 100 mL of 37° C. water, without agitation, and observing for mixing characteristics. Results varied from formation of oil drops on the top to separation of phases to uniform, but cloudy dispersions. Generally speaking, it is believed that formulations able to readily disperse in aqueous solution will have better dispersion characteristics upon administration. It was surprisingly found, however, as shown below in Examples 7-9, that formulations that did not readily disperse in aqueous solution (e.g., Formulation 13) and instead formed two phases upon introduction to the aqueous solution were found to be the most effective when administered to the human body.

### Example 2: Delivery Vehicle

In embodiments, the pharmaceutical composition is delivered in a gelatin capsule delivery vehicle. The gelatin capsule delivery vehicle includes, for example, gelatin (e.g., Gelatin, NF (150 Bloom, Type B)), hydrolyzed collagen (e.g., GELITA®, GELITA AG, Eberbach, Germany), glycerin, sorbitol special, or other excipients in proportions that are well known and understood by persons of ordinary skill in the art. Sorbitol special may be obtained commercially and may tend to act as a plasticizer and humectant.

A variety of delivery vehicles were developed, as show in Table 2, Gels A through F. In Table 2, each delivery vehicle A through F differs in the proportion of one or more components.

TABLE 2
Gelatin Capsule Delivery Vehicles

| | A | B | C | D | E | F |
|---|---|---|---|---|---|---|
| Ingredient | % w/w | % w/w | % w/w | % w/w | % w/w | % w/w |
| Gelatin, NF (150 Bloom, Type B) | 41.0 | 41.0 | 41.0 | 41.0 | 43.0 | 43.0 |
| Glycerin 99.7%, USP | 6.0 | 6.0 | 6.0 | 6.0 | 18.0 | 18.0 |
| Sorbitol Special, USP | 15.0 | 15.0 | 15.0 | 15.0 | | |
| GELITA ® (hydrolyzed collagen) | 3 | | | | 3.0 | |
| Citric acid | | 0.1 | 0.5 | 1 | | 0.1 |
| Purified Water | 35.0 | 37.9 | 37.5 | 37.0 | 36.0 | 38.9 |
| Total | 100.0 | 100.0 | 100.0 | 100.0 | 100.0 | 100.0 |
| Dissolution gel strips, Avg of 3 | 48 min | 50 min | 75 min | 70 min | | |
| (500 mL DH2O, 50 rpm @ 37° C.) | (42, 45, 58) | (50, 51, 50) | (76, 75, 74) | (70, 71, 70) | | |
| Dissolution gel strips, Avg of 3 | 70 min | | | | 78 min | 82 min |
| (500 mL pH 4 buffer, 50 rpm @ 37° C.) | | | | | | |

Each delivery vehicle A through F was prepared at a temperature range from about 45° C. to about 85° C. Each molten delivery vehicle A through F was cast into a film, dried, and cut into strips. The strips were cut into uniform pieces weighing about 0.5 g, with about 0.5 mm thickness. Strips were placed into a USP Type 2 dissolution vessel in either water or pH 4 buffer solution and the time for them to completely dissolve was recorded (see Table 2). Delivery vehicle A had the fastest dissolution in both water and pH 4 buffer solution.

### Example 3: Pharmaceutical Compositions and Delivery Vehicle

Various combinations of the pharmaceutical compositions from Table 1 and from Table 2 were prepared. The combinations are shown in Table 3.

TABLE 3

| | | | Batch | Delivery |
|---|---|---|---|---|
| Trial | Pharmaceutical Composition | Ratio | Size g | Vehicle |
| 1 | MCM:39/01 | 8:2 | 750 | A |
| 2 | MCM:50/13 | 8:2 | 750 | A |
| 3 | MCM:TEFOSE 63 | 8:2 | 750 | A |
| 4 | MCM:TEFOSE 63 | 8:2 | 750 | B |
| 5 | MIGLYOL 812:TEFOSE 63 | 9:1 | 750 | A |

Each aliquot of the pharmaceutical compositions of Table 3 about 300 mg to about 310 mg. Batch size was as listed in Table 3. To encapsulate the vehicle system, each 300 mg to about 310 mg pharmaceutical composition aliquot was encapsulated in about 200 mg of the gelatin capsule delivery vehicle. Thus, for example, in Trial 1, the pharmaceutical composition denoted by MCM:39/01 was encapsulated in gelatin capsule delivery vehicle A for a total encapsulated weight of about 500 mg to about 510 mg. The aliquot size is arbitrary depending on the concentration of the estradiol and the desired gelatin capsule delivery vehicle size. Artisans will readily understand how to adjust the amount of estradiol in the pharmaceutical composition to accommodate a given size of delivery vehicle, when the delivery vehicle encapsulates the pharmaceutical composition.

### Example 4: Estradiol Solubility

In various experiments, solubilizing agents were tested to determine whether they were able to solubilize 2 mg of estradiol for a total pharmaceutical composition weight of 100 mg. The solubilizing agents were considered suitable if estradiol solubility in the solubilizing agent was greater than or equal to about 20 mg/g. Initial solubility was measured by dissolving micronized estradiol into various solubilizing agents until the estradiol was saturated (the estradiol/solubilizing agent equilibrated for three days), filtering the undissolved estradiol, and analyzing the resulting pharmaceutical composition for estradiol concentration by HPLC.

TABLE 4
Solubility of Solubilizing Agents (*denotes literature reference)

| Ingredient | Solubility (mg/g) |
|---|---|
| PEG 400 | 105* |
| Propylene Glycol | 75* |
| Polysorbate 80 | 36* |
| TRANSCUTOL HP | 141 |
| CAPMUL PG8 | 31.2 |

### Example 5: Pharmaceutical Compositions

The following pharmaceutical compositions are contemplated.

| | | Qty/Batch |
|---|---|---|
| Ingredient | % w/w | (kg) |
| Gelatin 150 Bloom Limed Bone, NF | 41.00 | 82.00 |
| Hydrolyzed Gelatin | 3.00 | 6.00 |
| Glycerin 99.7% | 6.00 | 12.00 |
| Sorbitol Special, NF | 15.00 | 30.00 |
| Opatint White G-18006 | 1.20 | 2.40 |
| Opatine Red DG-15001 | 0.06 | 0.12 |
| Purified Water, USP | 33.74 | 67.48 |
| Total | 100.00 | 200.00 Kg |

| | Qty/ | | | |
|---|---|---|---|---|
| | Capsule | | Qty/ | |
| Ingredients | (mg) | % w/w | Batch | |
| Estradiol hemihydrate micronized, | 0.010 | 0.003 | 0.10 | g |
| USP | | | | |
| CAPMUL ® MCM, NF (Glyceryl | 240.0 | 79.997 | 2.40 | kg |
| Caprylate/Caprate or Medium Chain | | | | |
| Mono- and Diglycerides) | | | | |
| GELUCIRE ® 50/13 (stearoyl | 60.0 | 20.0 | 600.0 | g |
| polyoxyl-32 glycerides NF) | | | | |
| Total | 300.0 | 100.0 | 3.0 | kg |

| | Qty/ | | | |
|---|---|---|---|---|
| | Capsule | | Qty/ | |
| Ingredients | (mg) | % w/w | Batch | |
| Estradiol hemihydrate micronized, | 0.010 | 0.003 | 0.10 | g |
| USP | | | | |
| MIGLOYL ® 812 (medium chain | 270.0 | 89.997 | 2.70 | kg |
| triglyceride) | | | | |
| TEFOSE ® 63 (mixture of PEG-6 | 30.0 | 10.0 | 300.0 | g |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3.00 | kg |

| | Qty/ | | | |
|---|---|---|---|---|
| | Capsule | | Qty/ | |
| Ingredients | (mg) | % w/w | Batch | |
| Estradiol hemihydrate micronized, | 0.026* | 0.009 | 0.26 | g |
| USP | | | | |
| MIGLOYL ® 812 (medium chain | 270.0 | 89.991 | 2.70 | kg |
| triglyceride) | | | | |
| TEFOSE ® 63 (mixture of PEG-6 | 30.02 | 10.0 | 300.0 | g |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3.00 | kg |

*1.0 mg estradiol is equivalent to 1.03 mg estradiol hemihydrate

| | | | Qty/ | |
|---|---|---|---|---|
| | Qty/ | | Batch | |
| | Capsule | | (alternate | |
| Ingredients | (mg) | % w/w | batch size) | |
| Estradiol hemihydrate | 0.0041* | 0.001 | 0.041 | g |
| micronized, USP | | | (0.615 | g) |
| MIGLOYL ® 812 (medium | 269.99 | 89.999 | 2700.0 | g |
| chain triglyceride) | | | (40.50 | kg) |
| TEFOSE ® 63 (mixture | 30.0 | 10.0 | 300.0 | g |
| of PEG-6 stearate | | | (4.50 | kg) |
| or ethylene glycol | | | | |
| palmitostearate or | | | | |
| PEG-32 stearate; | | | | |
| polyoxyl 6 and | | | | |
| polyoxyl 32 | | | | |
| palmitostearate/ | | | | |
| glycol stearate) | | | | |
| Total | 300.0 | 100.0 | 3000.0 | g |
| | | | 45.0 | kg |

*1.0 mg estradiol is equivalent to 1.03 mg estradiol hemihydrate

| | Qty/ | | | |
|---|---|---|---|---|
| | Capsule | | Qty/ | |
| Ingredients | (mg) | % w/w | Batch | |
| Estradiol hemihydrate micronized, | 0.0103* | 0.003 | 1.545 | g |
| USP | | | | |
| MIGLOYL ® 812 (medium chain | 269.99 | 89.997 | 40.5 | kg |
| triglyceride) | | | | |
| TEFOSE ® 63 (mixture of PEG-6 | 30.0 | 10.0 | 4.50 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 45.00 | kg |

*1.0 mg estradiol is equivalent to 1.03 mg estradiol hemihydrate

| | Qty/ | | | |
|---|---|---|---|---|
| | Capsule | | Qty/ | |
| Ingredients | (mg) | % w/w | Batch | |
| Estradiol hemihydrate micronized, | 0.026* | 0.009 | 3.90 | g |
| USP | | | | |
| MIGLOYL ® 812 (medium chain | 269.97 | 89.991 | 40.50 | kg |
| triglyceride) | | | | |
| TEFOSE ® 63 (mixture of PEG-6 | 30.0 | 10.0 | 4.50 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 45.00 | kg |

*1.0 mg estradiol is equivalent to 1.03 mg estradiol hemihydrate

| | Qty/ | | | |
|---|---|---|---|---|
| | Capsule | | Qty/ | |
| Ingredients | (mg) | % w/w | Batch | |
| Estradiol hemihydrate micronized, | 0.00 | 0.00 | 0.00 | g |
| USP | | | | |
| MIGLOYL ® 812 (medium chain | 270.0 | 90.0 | 40.5 | kg |
| triglyceride) | | | | |
| TEFOSE ® 63 (mixture of PEG-6 | 30.0 | 10.0 | 4.5 | kg |
| stearate or ethylene glycol | | | | |
| palmitostearate or PEG-32 stearate; | | | | |
| polyoxyl 6 and polyoxyl 32 | | | | |
| palmitostearate/glycol stearate) | | | | |
| Total | 300.0 | 100.0 | 3000.0 | g |

In the Examples below, TX-004HR is Pharmaceutical Compositions 4, 5, and 6 (TX-004HR 4 μg, TX-004HR 10 μg, and TX-004HR 25 μg) compared to Pharmaceutical Composition 7.

### Example 6: Process

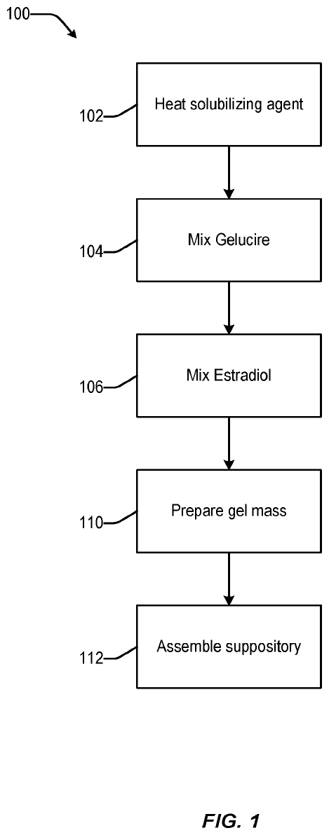

FIG. 1 illustrates an embodiment of a method making pharmaceutical composition comprising estradiol solubilized in CapmulMCM/Gelucire solubilizing agent encapsulated in a soft gelatin delivery vehicle 100. In operation 102, the CapmulMCM is heated to 40° C.±5° C. Heating may be accomplished through any suitable means. The heating may be performed in any suitable vessel, such as a stainless steel vessel. Other pharmaceutical compositions can be made using the same general method by substituting various excipients, including the solubilizing agent.

In operation 104, GELUCIRE is mixed with the CapmulMCM to form the finished solubilizing agent. As used herein, any form of GELUCIRE may be used in operation 104. For example, one or more of GELUCIRE 39/01, GELUCIRE 43/01, GELUCIRE 50/13 may be used in operation 104. Mixing is performed as would be known to persons of ordinary skill in the art, for example by impeller, agitator, stirrer, or other like devices used to mix pharmaceutical compositions. Operation 104 may be performed under an inert or relatively inert gas atmosphere, such as nitrogen gas. Mixing may be performed in any vessels that are known to persons of ordinary skill in the art, such as a stainless steel vessel or a steel tank.

In operation 106 estradiol is mixed into the solubilizing agent. In embodiments, the estradiol in micronized when mixed into the solubilizing agent. In other embodiments, the estradiol added is in a non-micronized form. Mixing may be facilitated by an impeller, agitator, stirrer, or other like devices used to mix pharmaceutical compositions. Operation 106 may be performed under an inert or relatively inert gas atmosphere, such as nitrogen gas.

In embodiments, however, the addition of estradiol may be performed prior to operation 104. In that regard, operations 104 and 106 are interchangeable with respect to timing or can be performed contemporaneously with each other.

In operation 110, the gelatin delivery vehicle is prepared. Any of the gelatin delivery vehicles described herein may be used in operation 110. In embodiments, gelatin, hydrolyzed collagen, glycerin, and other excipients are combined at a temperature range from about 45° C. to about 85° C. and prepared as a film. Mixing may occur in a steel tank or other container used for preparing gelatin delivery vehicles. Mixing may be facilitated by an impellor, agitator, stirrer, or other devices used to combine the contents of gelatin delivery vehicles. Operation 110 may be performed under an inert or relatively inert gas atmosphere, such as nitrogen gas. In embodiments, the gelatin delivery vehicle mixture is degassed prior to being used to encapsulate the pharmaceutical composition.

In operation 112, the gelatin delivery vehicle encapsulates the pharmaceutical composition, according to protocols well known to persons of ordinary skill in the art. In operation 112, a soft gelatin capsule delivery vehicle is prepared by combining the pharmaceutical composition made in operation 106 with the gelatin delivery vehicle made in operation 110. The gelatin may be wrapped around the material, partially or fully encapsulating it or the gelatin can also be injected or otherwise filled with the pharmaceutical composition made in operation 106.

In embodiments, operation 112 is completed in a suitable die to provide a desired shape. Vaginal soft gel capsules may be prepared in a variety of geometries. For example, vaginal soft gel capsules may be shaped as a tear drop, a cone with frustoconical end, a cylinder, a cylinder with larger “cap” portion as illustrated in FIG. 2, or other shapes suitable for insertion into the vagina. The resulting pharmaceutical composition encapsulated in the soft gelatin delivery vehicle may be inserted digitally or with an applicator.

### Example 7: Study of Estradiol Pharmaceutical Composition on the Improvement of Vulvovaginal Atrophy (VVA)

The objective of this study was designed to evaluate the efficacy and safety of a pharmaceutical composition comprising 10 μg estradiol (i.e., Pharmaceutical Composition 2) in treating moderate to severe symptoms of VVA associated with menopause after 14 days of treatment, and to estimate the effect size and variability of vulvovaginal atrophy endpoints. In addition, the systemic exposure to estradiol from single and multiple doses of the pharmaceutical composition was investigated.

This study was a phase 1, randomized, double-blind, placebo-controlled trial to evaluate safety and efficacy of the pharmaceutical composition in reducing moderate to severe symptoms of vaginal atrophy associated with menopause and to investigate the systemic exposure to estradiol following once daily intravaginal administrations of a pharmaceutical composition for 14 days.

Postmenopausal subjects who met the study entry criteria were randomized to one of two treatment groups (pharmaceutical composition or placebo). During the screening period subjects were asked to self-assess the symptoms of VVA, including vaginal dryness, vaginal or vulvar irritation or itching, dysuria, vaginal pain associated with sexual activity, and vaginal bleeding associated with sexual activity. Subjects with at least one self-assessed moderate to severe symptom of VVA identified by the subject as being most bothersome to her were eligible to participate in the study.

Clinical evaluations were performed at the following time points:
Screening Period (up to 28 days);
Visit 1—Randomization/Baseline (day 1);
Visit 2—Interim (day 8); and
Visit 3—End of the treatment (day 15).

Eligible subjects were randomized in a 1:1 ratio to receive either pharmaceutical composition comprising estradiol 10 μg or a matching placebo vaginal softgel capsule, and self-administered their first dose of study medication at the clinical facility under the supervision of the study personnel. Serial blood samples for monitoring of estradiol level were collected at 0.0, 1.0, 3.0, and 6.0 hours relative to first dose administration on day 1. Subjects remained at the clinical site until completion of the 6-hour blood draw and returned to clinical facility for additional single blood draws for measurement of estradiol concentration on day 8 (before the morning dose) and day 15. Subjects were provided with enough study medication until the next scheduled visit and were instructed to self-administer their assigned study treatment once a day intravaginally at approximately the same time (±1 hour) every morning. Each subject was provided with a diary in which she was required to daily record investigational drug dosing dates and times. Subjects returned to clinical facility on day 8 for interim visit and on day 15 for end of treatment assessments and post study examinations. Capsule disintegration state was assessed by the investigator at day 1 (6 hours post-dose) and day 15.

The study involved a screening period of up to 28 days before randomization and treatment period of 14 days. Selection of dosage strength (estradiol 10 μg) and treatment regimen (once daily for two weeks) was based on the FDA findings on safety and efficacy of the RLD.

### Number of Subjects (Planned and Analyzed)

Up to 50 (25 per treatment group) postmenopausal female subjects 40 to 75 years old with symptoms of moderate to severe VVA were randomized. 50 subjects were enrolled, 48 subjects completed the study, and 48 subjects were analyzed.

### Diagnosis and Main Criteria for Inclusion

Fifty female subjects were enrolled in the study. Post-menopausal female subjects 40 to 75 years of age, with a mean age was 62.3 years were enrolled. Subjects' mean weight (kg) was 71.2 kg with a range of 44.5-100 kg. Subjects' mean height (cm) was 162.6 cm with a range of 149.9-175.2 cm, and the mean BMI (kg/m²) was 26.8 kg/m² with a range of 19-33 kg/m². Criteria of inclusion in the study included: self-identification of at least one moderate to severe symptom of VVA, for example, vaginal dryness, dyspareunia, vaginal or vulvar irritation, burning, or itching, dysuria, vaginal bleeding associated with sexual activity, that was identified by the subject as being most bothersome to her; ≤5% superficial cells on vaginal smear cytology; vaginal pH>5.0; and estradiol level ≤50 pg/mL. Subject who were judged as being in otherwise generally good health on the basis of a pre-study physical examination, clinical laboratory tests, pelvic examination, and mammography were enrolled.

### Estradiol 10 μg or Placebo, Dose, and Mode of Administration

Subjects were randomly assigned (in 1:1 allocation) to self-administer one of the following treatments intravaginally once daily for 14 days:
Treatment A: The pharmaceutical composition of Example 5 (Pharmaceutical Composition 2: 10 μg estradiol); or
Treatment B: Placebo vaginal softgel capsule, containing the same formulation as Treatment A, except for the 10 μg of estradiol.

The estradiol formulation was a tear drop shaped light pink soft gel capsule. Treatment B had the same composition, appearance, and route of administration as the Treatment A, but contained no estradiol.

### Duration of Treatment

The study involved a screening period of up to 28 days before randomization and a treatment period of 14 days.

### Criteria for Evaluation

Efficacy Endpoints:
Change from baseline (screening) to day 15 in the Maturation Index (percent of parabasal vaginal cells, superficial vaginal cells, and intermediate vaginal cells) of the vaginal smear. Data for this endpoint are shown in Tables 6-8.
Change from baseline (screening) to day 15 in vaginal pH. Data for this endpoint are shown in Table 9.
Change from baseline (randomization) to day 15 in severity of the most bothersome symptoms: (1) vaginal dryness; (2) vaginal or vulvar irritation, burning, or itching; (3) dysuria; (4) dyspareunia; (5) vaginal bleeding associated with sexual activity. Data for this endpoint are shown in Tables 13 and 15.
Change from baseline (randomization) to day 15 in investigator's assessment of the vaginal mucosa. Data for this endpoint are shown in Tables 18-21.

Unless otherwise noted, the efficacy endpoints were measured as a change-from Visit 1—Randomization/Baseline (day 1) to Visit 3—End of the treatment (day 15), except for vaginal bleeding which was expressed as either treatment success or failure.

Other endpoints include:
Vital signs, weight, changes in physical exam, pelvic and breast exam, and adverse events were evaluated as part of the safety endpoints.
Concentration of estradiol at each sampling time.
Peak concentration of estradiol on day 1 and sampling time at which peak occurred.
Delivery vehicle disintegration to measure the amount of residual delivery vehicle remains in the vagina post treatment.

Results from the assessment of plasma concentrations of estradiol are presented in Table 5.

TABLE 5
Safety Results: The descriptive statistics for Day 1 plasma estradiol
Cₘₐₓ and Tₘₐₓ are provided below.

| | Estradiol 10 μg | | Placebo | |
|---|---|---|---|---|
| | Cₘₐₓ | Tₘₐₓ | Cₘₐₓ | Tₘₐₓ |
| N | 24 | 24 | 26 | 26 |
| Mean ± SD | 30.7 ± 7.47 | 2.12 ± 1.73 | 27.5 ± 17.26 | 4.00 ± 2.68 |
| Geometric Mean | 29.9 | — | 24.7 | — |
| Median | 29.8 | 1.00 | 22.1 | 6.00 |
| Min, Max | 19.7, 52.3 | 1.00, 6.00 | 15.1, 90.0 | 0.00, 6.00 |
| CV % | 24.3% | 81.3% | 62.9% | 67.1% |

### Maturation Index Results

Vaginal cytology data was collected as vaginal smears from the lateral vaginal walls according to standard procedures to evaluate vaginal cytology at screening and Visit 3—End of treatment (day 15). The change in the Maturation Index was assessed as a change in cell composition measured at Visit 1—Baseline (day 1) compared to the cell composition measured at Visit 3—End of treatment (day 15). The change in percentage of superficial, parabasal, and intermediate cells obtained from the vaginal mucosal epithelium from a vaginal smear was recorded. Results from these assessments are presented in Tables 6, 7, and 8.

TABLE 6
Primary Efficacy Analysis Results of Change from Baseline (Screening) to
Day 15 in the Maturation Index of the Vaginal Smear (Percent Parabasal Cells)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference | P-value |
| Intent- | N | 24 | 24 | — | — | — |
| to-Treat | | | | | | |
| | Least- | −54.4 | −4.80 | −49.6 | (−60.4, −38.8) | <0.0001 |
| | Squares | | | | | |
| | Mean | | | | | |
| | Mean ± SD | −53.8 ± 39.7 | −5.4 ± 22.3 | — | — | — |
| | Median | −60.0 | −5.0 | — | — | — |
| | Min, Max | −100.0, 0.0 | −60.0, 60.0 | — | — | — |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 7
Primary Efficacy Analysis Results of Change from Baseline (Screening) to Day 15 in
the Maturation Index of the Vaginal Smear (Superficial Cells)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference | P-value |
| Intent- | N | 24 | 24 | — | — | — |
| to-Treat | | | | | | |
| | Least- | 35.2 | 8.75 | 26.5 | (15.4, 37.6) | 0.0002 |
| | Squares | | | | | |
| | Mean | | | | | |
| | Mean ± SD | 35.2 ± 26.4 | 8.8 ± 18.7 | — | — | — |
| | Median | 40.0 | 0.0 | — | — | — |
| | Min, Max | 0.0, 80.0 | 0.0, 90.0 | — | — | — |

¹Confidence interval for the estradiol 10 μg-Placebo from ANOVA with treatment as a fixed effect.
²P-value for treatment comparison from ANOVA with treatment as a fixed effect.

TABLE 8
Primary Efficacy Analysis Results of Change from Baseline (Screening)
to Day 15 in the Maturation Index of the Vaginal Smear (Intermediate Cells)

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference | P-value² |
| Intent- | N | 24 | 24 | — | — | — |
| to-Treat | | | | | | |
| | Least- | 18.7 | −3.54 | 22.3 | (11.1, 33.5) | 0.0017 |
| | Squares | | | | | |
| | Mean | | | | | |
| | Mean ± SD | 18.5 ± 42.7 | −3.3 ± 21.6 | — | — | — |
| | Median | 22.5 | −5.0 | — | — | — |
| | Min, Max | −60.0, 100.0 | −60.0, 20.0 | — | — | — |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

### Change in pH Results

Vaginal pH was measured at Screening and Visit 3—End of treatment (day 15). The pH measurement was obtained by pressing a pH indicator strip against the vaginal wall. The subjects entering the study were required to have a vaginal pH value greater than 5.0 at screening. pH values were recorded on the subject's case report form. The subjects were advised not to have sexual activity and to refrain from using vaginal douching within 24 hours prior to the measurement. Results from these assessments are presented in Table 9.

TABLE 9
Primary Efficacy Analysis Results of Change from
Baseline (Screening) to Day 15 in Vaginal pH

| | | | | Difference | | Estradiol |
|---|---|---|---|---|---|---|
| | | | | Between | 90% | 10 μg vs. |
| | | Estradiol | | Treatment | CI for | Placebo |
| Population | Statistics | 10 μg | Placebo | Means | Difference | P-value² |
| Intent- | N | 24 | 24 | — | — | — |
| to-Treat | | | | | | |
| | Least- | −0.974 | −0.339 | −0.635 | (−0.900, | 0.0002 |
| | Squares | | | | −0.368) | |
| | Mean | | | | | |
| | Mean ± SD | −0.917 ± 0.686 | −0.396 ± 0.659 | — | — | — |
| | Median | −1.00 | −0.500 | — | — | — |
| | Min, Max | −2.00, 0.500 | −1.50, 0.500 | — | — | — |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

### Most Bothersome Symptoms Data

Subjects were asked to specify the symptom that she identified as the “most bothersome symptom.” During the screening period all of the subjects were provided with a questionnaire to self-assess the symptoms of VVA: (1) vaginal dryness; (2) vaginal or vulvar irritation, burning, or itching; (3) dysuria; (4) dyspareunia; (5) vaginal bleeding associated with sexual activity. Each symptom, with the exception of vaginal bleeding associated with sexual activity, was measured on a scale of 0 to 3, where 0=none, 1=mild, 2=moderate, and 3=severe. Vaginal bleeding associated with sexual activity was measured in a binary scale: N=no bleeding; Y=bleeding. The subject's responses were recorded. All randomized subjects were also provided a questionnaire to self-assess the symptoms of VVA at Visit 1—Randomization/Baseline (day 1) and at Visit 3—End of the treatment (day 15). Subjects recorded their self-assessments daily in a diary and answers were collected on days 8 and 15 (end of treatment). Pre-dose evaluation results obtained at Visit 1 were considered as baseline data for the statistical analyses. Data from these assessments are presented in Tables 10 and 11.

TABLE 10
Baseline Characteristics for Vaginal Atrophy Symptoms (ITT Population)

| | | | | Estradiol |
|---|---|---|---|---|
| | | | | 10 μg vs. |
| | | Estradiol | | Placebo |
| VVA Symptom | Statistics | 10 μg | Placebo | P-value¹ |
| Vaginal dryness | N of Subjects | 24 | 24 | — |
| | Mean | 2.292 | 2.375 | 0.68231 |
| Vaginal or vulvar | N of Subjects | 24 | 24 | — |
| irritation/burning/ | Mean | 0.875 | 1.333 | 0.08721 |
| itching | | | | |
| Pain, burning or | N of Subjects | 24 | 24 | — |
| stinging when | Mean | 0.583 | 0.625 | 0.87681 |
| urinating | | | | |
| Vaginal pain | N of Subjects² | 12 | 12 | — |
| associated with | Mean | 2.083 | 2.333 | 0.54281 |
| sexual activity | | | | |
| Vaginal bleeding | N of Subjects² | 12 | 12 | |
| associated with | Percent³ | 25.00 | 33.33 | 0.31463 |
| sexual activity | | | | |

1P-value for treatment comparison from ANOVA/ANCOVA with treatment as a fixed effect and Baseline as a covariate when appropriate.
²N = number of subjects sexually active at baseline.
³Percent of subjects with bleeding, evaluated using Fisher's Exact Test.

TABLE 11
Additional Efficacy Analysis Results of Change from Baseline (Randomization) to
Day 15 in Severity of Vaginal Atrophy Symptoms

| | | | | Difference | | |
|---|---|---|---|---|---|---|
| | | Least-Squares Mean | | Between | | Estradiol 10 μg |
| | Statistical | Estradiol | | Treatment | 90% CI for | vs. Placebo P- |
| Symptom | Method¹ | 10 μg | Placebo | Means | Difference² | value |
| Vaginal dryness | ANCOVA | 0.980 | 0.729 | 0.251 | −0.706, 0.204) | 0.3597 |
| Vaginal or vulvar | ANCOVA | 0.694 | 0.514 | 0.180 | −0.549, 0.189) | 0.4159 |
| Irritation/burning/ | | | | | | |
| itching | | | | | | |
| Pain/Burning/ | ANCOVA | 0.391 | 0.359 | 0.032 | −0.263, 0.200) | 0.8185 |
| Stinging | | | | | | |
| (Urination) | | | | | | |
| Vaginal pain | ANOVA | 0.800 | 0.500 | 0.300 | −1.033, 0.433) | 0.4872 |
| associated with | | | | | | |
| sexual activity | | | | | | |

¹ANOVA model contained a fixed effect for treatment. ANCOVA added baseline as a covariate to the model.
²Confidence interval for the difference between estradiol 10 μg and Placebo treatment least-squares means.

Changes to the most bothersome symptom from the baseline was scored according to the evaluation of VVA symptoms generally set forth above. Tables 13 and 14 show a comparison between the pharmaceutical composition 1 and placebo generally for most bothersome symptom and vaginal atrophy symptom. It is noteworthy to point out that these measurement demonstrated a trend of improvement, though not statistically significant, at day 15.

TABLE 13
Primary Efficacy Analysis Results of Change from Baseline
(Randomization) to Day 15 in Severity of the Most Bothersome VVA

| | | | | Difference | 90% | Estradiol |
|---|---|---|---|---|---|---|
| | | | | Between | CI for | 10 μg vs. |
| Pop- | Sta- | Estradiol | | Treatment | Differ- | Placebo |
| ulation | tistics | 10 μg | Placebo | Means | ence¹ | P-value² |
| Intent- | N | 24 | 24 | — | — | — |
| to-Treat | | | | | | |
| | Least | −1.043 | −1.042 | −0.002 | (−0.497, | 0.9951 |
| | Squares | | | | 0.493) | |
| | Mean | | | | | |
| | Mean ± | −1.043 ± | −1.042 ± | — | — | — |
| | SD | 0.928 | 1.08 | | | |
| | Median | −1.00 | −1.00 | — | — | — |
| | Min, | −3.00, | −3.00, | — | — | — |
| | Max | 0.00 | 0.00 | | | |

¹Confidence interval for the estradiol 10 μg-Placebo from ANOVA with treatment as a fixed effect.
²P-value for treatment comparison from ANOVA with treatment as a fixed effect.

TABLE 14
Additional Efficacy Analysis Results of Change from Baseline (Randomization) to
Day 15 in Severity of Vaginal Atrophy Symptoms

| | | | | Difference | | TX42-004- |
|---|---|---|---|---|---|---|
| | | Least-Squares Mean | | Between | | HR vs. |
| | Statistical | TX-12-004- | | Treatment | 90% CI for | Placebo P- |
| Symptom | Method¹ | HR | Placebo | Means | Difference² | value |
| Dryness | ANCOVA | −0.980 | −0.729 | −0.251 | (−0.706, 0.204) | 0.3597 |
| Irritation | ANCOVA | −0.694 | −0.514 | −0.180 | (−0.549, 0.189) | 0.4159 |
| Pain (Sex) | ANOVA | −0.800 | −0.500 | −0.300 | (−1.033, 0.433) | 0.4872 |
| Pain/Burning/ | ANCOVA | −0.391 | −0.359 | −0.032 | (−0.263, 0.200) | 0.8185 |
| Stinging (Urination) | | | | | | |

¹ANOVA model contained a fixed effect for treatment. ANCOVA added baseline as a covariate to the model.
²Confidence interval for the difference between TX-12-004-HR and Placebo treatment least-squares means.

With respect to the most bothersome symptoms data presented in Tables 13 and 14, the period over which the data was measured is generally considered insufficient to make meaningful conclusions. However, the trends observed as part of this study suggest that the data will show improvement of the most bothersome symptoms when data for a longer time period is collected.

The absence or presence of any vaginal bleeding associated with sexual activity was also measured as one of the most bothersome symptoms. The data for vaginal bleeding associated with sexual activity is reported in Table 15.

TABLE 15
Primary Efficacy Analysis Results of Change from Baseline
(Randomization) to Day15 in Vaginal Bleeding Associated
with Sexual Activity

| | | Baseline (Randomization) and Day 15 | | | |
|---|---|---|---|---|---|
| | | Summary of Vaginal Bleeding | | | |
| | | | | | No |
| | | Bleeding/ | | No | Bleeding/ |
| | | No | Bleeding/ | Bleeding/ | No |
| | | Bleeding | Bleeding | Bleeding | Bleeding |
| Treatment | N* | (Success)² | (Failure) | (Failure) | (NC) |
| Estradiol 10 μg | 10 | 2 (100%) | 0 | 0 | 8 |
| Placebo | 10 | 1 (20%) | 3 | 1 | 5 |

| | | | | |
|---|---|---|---|---|
| P-Value for | 0.1429 | — | — | — |
| Estradiol 10 μg vs. | | | | |
| Placebo¹ | | | | |

*N = Total number of patients within each treatment group who were sexually active at both Baseline and Day 15 and provided a response at both visits.
NC = No Change—not considered in the statistical comparison.
¹P-value for treatment comparison from Fisher's Exact Test.
²Percent is based on the number of subjects classified as either a Success or a Failure (N = 2 for estradiol 10 μg; N = 5 for Placebo

### Estradiol Level/Pharmacokinetics Data

In this study, the systemic exposure to estradiol following once daily intravaginal administration of estradiol 10 μg for 14 days was investigated. Descriptive statistics of the plasma estradiol concentrations taken at each sampling time and the observed Cₘₐₓ and Tₘₐₓ values were recorded in Tables 16 and 17. No statistically significant difference in the systemic concentration of estradiol 10 μg versus the placebo group was observed, which suggests the estradiol is not carried into the blood stream where it will have a systemic effect. Rather, it remains in localized tissues; the effect of estradiol is therefore believed be local to the location of administration (i.e., the vagina). The lower limits of detection of the assays used to measure the pharmacokinetic data may have affected the measured the accuracy of the PK values presented. Additional PK studies were performed with more accurate assays in Examples 8 and 9.

For the purpose of monitoring the estradiol level during the study blood samples were collected at 0.0, 1.0, 3.0, and 6.0 hours relative to dosing on day 1; prior to dosing on day 8; and prior to dosing on day 15. Efforts were made to collect blood samples at their scheduled times. Sample collection and handling procedures for measurement of estradiol blood level was performed according to procedure approved by the sponsor and principal investigator. All baseline and post-treatment plasma estradiol concentrations were determined using a validated bioanalytical (UPLC-MS/MS) methods. These data are shown in Tables 16 and 17.

TABLE 16
Descriptive Statistics of Estradiol Concentrations (pg/mL) at Each Sampling Time

| | Sampling Time | | | | | |
|---|---|---|---|---|---|---|
| | | | | | Pre-dose Day | Pre-dose Day |
| Treatment | 0 Hour | 1 Hour | 3 Hours | 6 Hours | 8 | 15 |
| Estradiol 10 μg | | | | | | |
| N | 24 | 24 | 24 | 24 | 24 | 22 |
| Mean ± SD | 20.1 ± 5.74 | 28.7 ± 5.89 | 25.7 ± 5.71 | 23.4 ± 7.91 | 21.4 ± 9.28 | 23.4 ± 8.72 |
| Median | 20.2 | 28.9 | 24.7 | 22.3 | 20.7 | 20.7 |
| Min, Max | 2.63, 38.3 | 18.8, 43.9 | 19.3, 47.5 | 3.31, 52.3 | 2.09, 52.2 | 17.9, 54.7 |
| Placebo | | | | | | |
| N | 26 | 26 | 26 | 26 | 25 | 24 |
| Mean ± SD | 20.5 ± 4.29 | 21.0 ± 6.14 | 19.0 ± 5.92 | 26.9 ± 17.36 | 29.9 ± 22.51 | 28.1 ± 16.80 |
| Median | 20.8 | 20.8 | 20.9 | 21.7 | 21.6 | 21.1 |
| Min, Max | 4.03, 29.1 | 3.19, 41.2 | 3.15, 26.9 | 15.1, 90.0 | 15.0, 116.2 | 14.7, 81.3 |

TABLE 17
Descriptive Statistics of Estradiol Cₘₐₓ aand Tₘₐₓ Day 1

| | Estradiol 10 μg | | Placebo | |
|---|---|---|---|---|
| | Cₘₐₓ | Tₘₐₓ | Cₘₐₓ | Tₘₐₓ |
| N | 24 | 24 | 26 | 26 |
| Mean ± SD | 30.7 ± 7.47 | 2.12 ± 1.73 | 27.5 ± 17.26 | 4.00 ± 2.68 |
| Geometric Mean | 29.9 | — | 24.7 | — |
| Median | 29.8 | 1.00 | 22.1 | 6.00 |
| Min, Max | 19.7, 52.3 | 1.00, 6.00 | 15.1, 90.0 | 0.00, 6.00 |
| CV % | 24.3% | 81.3% | 62.9% | 67.1% |

### Assessment of Vaginal Mucosa Data

The investigators rated the vaginal mucosal appearance at day 1 (pre-dose) and day 15. Vaginal color, vaginal epithelial integrity, vaginal epithelial surface thickness, and vaginal secretions were evaluated according to the following degrees of severity: none, mild, moderate, or severe using scales 0 to 3, where 0=none, 1=mild, 2=moderate, and 3=severe. Results from these investigators rated assessments are presented in Tables 18, 19, 20, and 21.

TABLE 18
Primary Efficacy Analysis Results of Change from Baseline
(Randomization) to Day 15 in Investigator's Assessment of the
Vaginal Mucosa (Assessment of Vaginal Color)

| | | | | Difference | 90% | Estradiol |
|---|---|---|---|---|---|---|
| | | | | Between | CI for | 10 μg vs. |
| Pop- | Sta- | Estradiol | | Treatment | Differ- | Placebo |
| ulation | tistics | 10 μg | Placebo | Means | ence¹ | P-value² |
| Intent- | N | 24 | 24 | — | — | — |
| to-Treat | | | | | | |
| | Least- | −0.199 | −0.009 | −0.191 | (−0.434, | 0.1945 |
| | squares | | | | 0.052) | |
| | Mean | | | | | |
| | Mean ± | −0.333 ± | 0.125 ± | | | |
| | SD | 0.565 | 0.741 | | | |
| | Median | 0.00 | 0.00 | — | — | — |
| | Min, | −2.00, | −1.00, | — | — | — |
| | Max | 0.00 | 2.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 19
Primary Efficacy Analysis Results of Change from Baseline
(Randomization) to Day 15 in Investigator's Assessment of the Vaginal
Mucosa (Assessment of Vaginal Epithelial Integrity)

| | | | | Difference | 90% | Estradiol |
|---|---|---|---|---|---|---|
| | | | | Between | CI for | 10 μg vs. |
| Pop- | Sta- | Estradiol | | Treatment | Differ- | Placebo |
| ulation | tistics | 10 μg | Placebo | Means | ence¹ | P-value² |
| Intent- | N | 24 | 24 | — | — | — |
| to-Treat | | | | | | |
| | Least- | −0.342 | 0.176 | −0.518 | (−0.726, | 0.0001 |
| | squares | | | | −0.311) | |
| | Mean | | | | | |
| | Mean ± | −0.417 ± | 0.250 ± | | | |
| | SD | 0.584 | 0.442 | | | |
| | Median | 0.00 | 0.00 | — | — | — |
| | Min, | −1.00, | 0.00, | — | — | — |
| | Max | 1.00 | 1.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 20
Primary Efficacy Analysis Results of Change from Baseline
(Randomization) to Day 15 in Investigator's Assessment of the
Vaginal Mucosa (Assessment of Vaginal Epithelial Surface Thickness)

| | | | | Difference | 90% | Estradiol |
|---|---|---|---|---|---|---|
| | | | | Between | CI for | 10 μg vs. |
| Pop- | Sta- | Estradiol | | Treatment | Differ- | Placebo |
| ulation | tistics | 10 μg | Placebo | Means | ence¹ | P-value² |
| Intent- | N | 24 | 24 | — | — | — |
| to-Treat | | | | | | |
| | Least- | −0.034 | −0.133 | 0.099 | (−0.024, | 0.1820 |
| | squares | | | | 0.221) | |
| | Mean | | | | | |
| | Mean ± | −0.125 ± | −0.042 ± | — | — | — |
| | SD | 0.338 | 0.550 | | | |
| | Median | 0.00 | 0.00 | — | — | — |
| | Min, | −1.00, | −1.00, | — | — | — |
| | Max | 0.00 | 1.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

TABLE 21
Primary Efficacy Analysis Results of Change from Baseline
(Randomization) to Day 15 in Investigator's Assessment of the
Vaginal Mucosa (Assessment of Vaginal Secretions)

| | | | | Difference | 90% | Estradiol |
|---|---|---|---|---|---|---|
| | | | | Between | CI for | 10 μg vs. |
| Pop- | Sta- | Estradiol | | Treatment | Dif- | Placebo |
| ulation | tistics | 10 μg | Placebo | Means | ference¹ | P-value² |
| Intent- | N | 24 | 24 | — | — | — |
| to-Treat | | | | | | |
| | Least- | −0.643 | −0.274 | −0.369 | (−0.661, | 0.0401 |
| | squares | | | | −0.076) | |
| | Mean | | | | | |
| | Mean ± | −0.792 ± | −0.125 ± | — | — | — |
| | SD | 0.779 | 0.741 | | | |
| | Median | −1.00 | 0.00 | — | — | — |
| | Min, | −2.00, 1.00 | −2.00, | — | — | — |
| | Max | | 2.00 | | | |

¹Confidence interval for the estradiol 10 μg-Placebo from ANCOVA with treatment as a fixed effect and baseline as a covariate.
²P-value for treatment comparison from ANCOVA with treatment as a fixed effect and baseline as a covariate.

### Delivery Vehicle Disintegration Data

Assessment of capsule disintegration in the vagina (presence or absence) at Day 1 (6 hours after dosing) and Day 15. Results of this assessment is presented in Table 22.

TABLE 22
Capsule Disintegration State in theVagina onDay 1 and Day15

| | Estradiol 10 μg | | Placebo | |
|---|---|---|---|---|
| | Day 1 | Day 15 | Day 1 | Day 15 |
| No evidence of | 23 (95.8%) | 24 (100.0%) | 26 (100.0%) | 24 (92.3%) |
| capsule present | | | | |
| Evidence of | 0 (0.0%) | 0 (0.0%) | 0 (0.0%) | 0 (0.0%) |
| capsule present | | | | |
| Assessment | 1 (4.2%) | 0 (0.0%) | 0 (0.0%) | 2 (7.7%) |
| not done | | | | |

Serum hormone level data was collected to measure the serum concentrations of estradiol. These data were used for screening inclusion and were determined using standard clinical chemistry methods,

### Appropriateness of Measurements

The selection of the efficacy measurements used in this study was based on FDA's recommendations for studies of estrogen and estrogen/progestin drug products for the treatment of moderate to severe vasomotor symptoms associated with the menopause and moderate to severe symptoms of vulvar and vaginal atrophy associated with the menopause (Food and Drug Administration, Guidance for Industry, Estrogen and Estrogen/Progestin Drug Products to Treat Vasomotor Symptoms and Vulvar and Vaginal Atrophy Symptoms—Recommendations for Clinical Evaluation. January 2003, hereby incorporated by reference).

Standard clinical, laboratory, and statistical procedures were utilized in the trial. All clinical laboratory procedures were generally accepted and met quality standards.

### Statistical Methods:

### Efficacy:

Analysis of variance (ANOVA) was used to evaluate the change from baseline differences between the subjects receiving estradiol 10 μg and placebo capsules for all efficacy endpoints, except for vaginal bleeding, to estimate the effect size and variability of the effect. In some cases, for example, for some vaginal atrophy symptoms, the change from baseline (post dose response) was correlated with the baseline value (p<0.05), so baseline was included as a covariate to adjust for this correlation (Analysis of Covariance, ANCOVA). The 90% confidence intervals on the differences between estradiol 10 μg and placebo endpoint means were determined to evaluate the effect size. The change from baseline in vaginal bleeding associated with sexual activity was evaluated in terms of the proportion of subjects who had treatment success or failure. Any subject reporting bleeding at baseline who did not report bleeding at Day 15 was considered to have been successfully treated. Any subject reporting bleeding at day 15 was considered a treatment failure, regardless of whether they reported baseline bleeding or not. Subjects reporting no bleeding at both baseline and day 15 were classified as no-change and were excluded from the statistical evaluation. The difference in the proportion of subjects with success between the two treatment groups was statistically evaluated using Fisher's Exact Test. Results of this difference in proportion are presented in Table 10.

### Measurements of Treatment Compliance

Subjects were required to complete a diary in order to record treatment compliance. Diaries were reviewed for treatment compliance at day 8 and day 15 visits. A total of 45 subjects (21 subjects in the estradiol 10 μg group and 24 subjects in the placebo group) were 100% compliant with the treatment regimen.

Due to the investigative nature of the study, no adjustments were made for multiplicity of endpoints.

### Safety:

The frequency and severity of all adverse events were summarized descriptively by treatment group.

Results: All forty eight (48) subjects who completed the study were included in the primary efficacy analyses. The results of efficacy analyses are presented throughout Tables 5, 6, and 7.

### Conclusions

### Efficacy

The two-week treatment with pharmaceutical composition 10 μg led to a statistically significant greater mean decrease in percent of parabasal cells than did placebo treatment (54% vs. 5%, p<0.0001), as illustrated in Table 6. At the same time, a significantly greater mean increase in the percent of superficial cells was observed with the pharmaceutical composition (35%) than with the placebo capsules (9%), with the difference being highly statistically significant (p=0.0002), as illustrated in Table 7. The difference in pH reduction between the pharmaceutical composition (0.97 units) compared to that for the placebo (0.34 units) was only slightly greater than 0.5 units, but the difference was detected as statistically significant (p=0.0002), as illustrated in Table 9.

While the decrease in severity of the most bothersome symptom was essentially the same (˜1 unit) for both pharmaceutical composition and placebo, the reductions in the severity of the individual symptoms of vaginal dryness, irritation and pain during sexual activity were all marginally better for the active treatment than for the placebo treatment. None of the differences between the two treatments, all of which were ≤0.3 units, were detected as statistically significant. There was no difference between the two treatments in regard to reduction of pain/burning/stinging during urination (˜0.4 unit reduction). The length of the study was not long enough to show a separation between the most bothersome symptoms in the pharmaceutical composition and placebo. However, the trends of most bothersome symptoms suggest that with a suitable period of time, significantly significant differences between the two treatments would be observed.

The two-week treatment with estradiol 10 μg capsules showed no statistically detectable difference in regard to reduction of severity from baseline according to the investigator's assessment of vaginal color or vaginal epithelial surface thickness. Pharmaceutical composition capsules did demonstrate a statistically significant greater reduction than did placebo in severity of atrophic effects on vaginal epithelial integrity (−0.34 vs. 0.18, p=0.0001) and vaginal secretions (−0.64 vs. −0.27, p=0.0401).

Descriptive statistical analyses (mean, median, geometric mean, standard deviation, CV, minimum and maximum, Cₘₐₓ, and Tₘₐₓ) were conducted on the estradiol concentrations at each sampling time, the peak concentration on day 1 and the time of peak concentration. Results from this assessment are presented in Tables 16 and 17.

A pharmaceutical composition comprising estradiol 10 μg outperformed placebo treatment in regard to improvement in the Maturation Index, reduction in vaginal pH, reduction in the atrophic effects on epithelial integrity and vaginal secretions. The lack of statistical significance between the two treatments in regard to reduction of severity for the most bothersome symptom, and the individual vaginal atrophy symptoms of dryness, irritation, pain associated with sexual activity, and pain/burning/stinging during urination, is not unexpected given the small number of subjects in the study and the short duration of therapy. Too few subjects in the study had vaginal bleeding associated with sexual activity to permit any meaningful evaluation of this vaginal atrophy symptom.

Of the 48 subjects enrolled in the study, 45 subjects were 100% compliant with the treatment regimen. Of the remaining three subjects, one removed herself from the study due to personal reasons and the other two subjects each missed one dose due to an adverse event.

### Safety

Although the Day 1 mean plasma estradiol peak concentration for the pharmaceutical composition was somewhat higher than that for the Placebo (ratio of geometric means=1.21: Test Product (estradiol 10 μg) 21%>Placebo), no statistically significant difference was determined. However, the assay methods were questionable, resulting in questionable PK data. Additional PK studies were performed in Examples 8 and 9.

There were no serious adverse events in the study.

Overall, the pharmaceutical composition comprising estradiol 10 μg was well tolerated when administered intravaginally in once daily regimen for 14 days.

### Example 8: PK Study (25 μg Formulation)

A PK study was undertaken to compare the 25 μg formulation disclosed herein (Pharmaceutical Composition 3) to the RLD. The results of the PK study for estradiol are summarized in Table 23. The p values for these data demonstrate statistical significance, as shown in Table 24.

TABLE 23
Statistical Summary of the Comparative Bioavailability Data
for Unscaled Average BE studies of Estradiol, Least Square
Geometric Means of Estradiol, Ratio of Means and 90%
Confidence Intervals, Fasting/Fed Bioequivalence Study
(Study No.: ESTR-1K-500-12), Dose 25 μg estradiol

| Parameter | Test | N | RLD | N | Ratio (%) | 90% C.I. |
|---|---|---|---|---|---|---|
| Cₘₐₓ | 23.0839 | 36 | 42.7024 | 36 | 54.06 | 44.18- |
| (pg/mL) | | | | | | 66.14 |
| AUC₀₋₂₄ | 89.2093 | 36 | 292.0606 | 36 | 30.54 | 23.72- |
| (pg · hr/mL) | | | | | | 39.34 |

TABLE 24
P-values for Table 23

| | P-Value | |
|---|---|---|
| Effect | Cₘₐₓ | AUC₀₋₂₄ |
| Treatment | <.0001 | <.0001 |
| Sequence | 0.4478 | 0.5124 |
| Period | 0.4104 | 0.7221 |

As illustrated in Table 23, baseline adjusted PK data illustrates that the formulations disclosed herein unexpectedly show a 54% decrease in Cₘₐₓ and a 31% decrease in the AUC relative to the RLD. This result is desirable because the estradiol is intended only for local absorption. These data suggest a decrease in the circulating levels of estradiol relative to the RLD. Moreover, it is noteworthy to point out that the Cₘₐₓ and AUC levels of estradiol relative to placebo are not statistically differentiable, which suggests that the formulations disclosed herein have a negligible systemic effect. As shown in Table 24, there was no significant difference between the test and reference products due to sequence and period effects. However, there was a significant difference due to treatment effect for both Cₘₐₓ and AUC.

Pharmacokinetics for circulating total estrone, a metabolite of estradiol, is show in Table 25. These data show that the total circulating estrone for the formulations disclosed herein resulted in a 55% decrease in the Cₘₐₓ for circulating estrone, and a 70% decrease in the AUC for circulating estrone.

TABLE 25
Statistical Summary of the Comparative Bioavailability Data for
Unscaled Average BE studies of Estrone, Least Square
Geometric Means, Ratio of Means and 90% Confidence
Intervals, Fasting/Fed Bioequivalence Study (Study No.:
ESTR-1K-500-12); Dose 25 μg estradiol

| Parameter | Test | N | RLD | N | Ratio (%) | 90% C.I. |
|---|---|---|---|---|---|---|
| Cₘₐₓ | 10.7928 | 36 | 23.5794 | 36 | 45.77 | 32.95 to |
| (pg/mL) | | | | | | 63.59 |
| AUC₀₋₂₄ | 51.2491 | 36 | 165.4664 | 36 | 30.97 | 19.8- |
| (pg · hr/mL) | | | | | | 48.45 |

TABLE 26
P-values for Table 25

| | P-Value | |
|---|---|---|
| Effect | Cₘₐₓ | AUC₀₋₂₄ |
| Treatment | 0.0002 | <.0001 |
| Sequence | 0.1524 | 0.464 |
| Period | 0.0719 | 0.0118 |

There was a significant difference between test and reference products due to treatment effect whereas there was no significant difference due to sequence and period effects for Cₘₐₓ. For AUC, there was a significant difference between test and reference products due to treatment, sequence, and period effects.

PK for circulating total estrone sulfate is shown in Table 27. These data show that the total circulating estrone sulfate for the pharmaceutical compositions disclosed herein resulted in a 33% decrease in the Cₘₐₓ and a 42% decrease in the AUC for circulating estrone sulfate.

TABLE 27
Statistical Summary of the Comparative Bioavailability Data for
Unscaled Average BE studies of Estrone Sulfate, Least Square
Geometric Means of Estrone Sulfate, Ratio of Means and 90%
Confidence Intervals, Fasting/Fed Bioequivalence Study (Study
No.: ESTR-1K-500-12); Dose 25 μg estradiol

| | | | | | Ratio | 90% |
|---|---|---|---|---|---|---|
| Parameter | Test | N | RLD | N | (%) | C.I. |
| Cₘₐₓ | 490.0449 | 36 | 730.5605 | 36 | 67.08 | 53.84- |
| (pg/mL) | | | | | | 83.57 |
| AUC₀₋₂₄ | 4232.9914 | 36 | 7323.0827 | 36 | 57.80 | 43.23- |
| (pg · | | | | | | 77.29 |
| hr/mL) | | | | | | |

TABLE 28
P-values for Table 27

| | P-Value | |
|---|---|---|
| Effect | Cₘₐₓ | AUC₀₋₂₄ |
| Treatment | 0.0042 | 0.0031 |
| Sequence | 0.5035 | 0.9091 |
| Period | 0.1879 | 0.8804 |

There was a significant difference between test and reference products due to treatment effect whereas there was no significant difference due sequence and period effects for both Cₘₐₓ and AUC.

### Example 9: PK Study (10 μg Formulation)

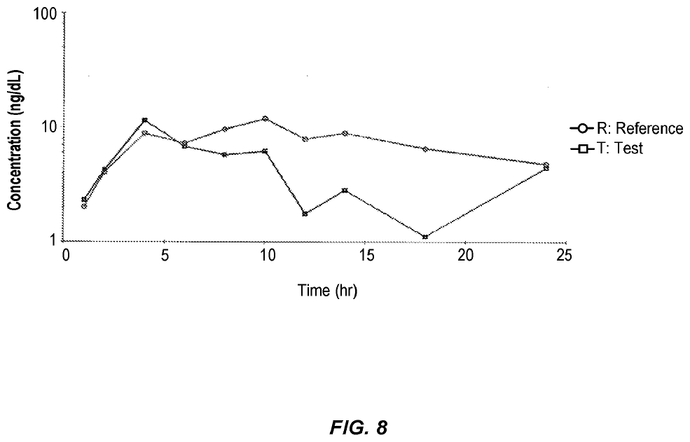

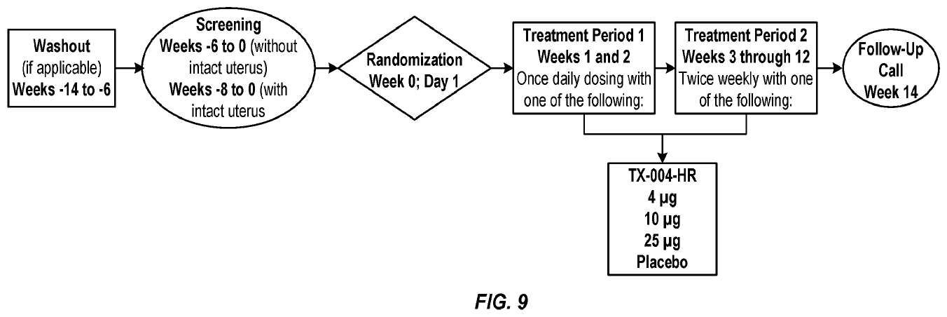

A PK study was undertaken to compare the 10 μg formulation disclosed herein (Pharmaceutical Composition 2) to the RLD. The results of the PK study for estradiol are summarized in Table 29-40, and FIGS. 9-14.

A PK study was undertaken to compare pharmaceutical compositions disclosed herein having 10 μg of estradiol to the RLD. The results of the PK study for estradiol are summarized in Tables 29-34, which demonstrate that the pharmaceutical compositions disclosed herein more effectively prevented systemic absorption of the estradiol. Table 35 shows that the pharmaceutical compositions disclosed herein had a 28% improvement over the RLD for systemic blood concentration Cₘₐₓ and 72% AUC improvement over the RLD.

TABLE 29
Summary of Pharmacokinetic Parameters of Test product (T) of
Estradiol—Baseline adjusted (N = 34)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 15.7176 ± | 50.3761 | 13.9000 | 6.5000 | 49.6000 |
| (pg/mL) | 7.9179 | | | | |
| AUC₀₋₂₄ | 53.0100 ± | 36.9041 | 49.9750 | 24.3000 | 95.1500 |
| (pg · hr/ | 19.5629 | | | | |
| mL) | | | | | |
| tₘₐₓ (hr) | 1.98 ± | 65.34 | 2.00 | 1.00 | 8.05 |
| | 1.29 | | | | |

TABLE 30
Summary of Pharmacokinetic Parameters of Reference product (R) of
Estradiol—Baseline adjusted (N = 34)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 24.1882 ± | 49.2877 | 24.1500 | 1.0000 | 55.3000 |
| (pg/mL) | 11.9218 | | | | |
| AUC₀₋₂₄ | 163.8586 ± | 43.9960 | 158.0375 | 2.0000 | 304.8500 |
| (pg · hr/ | 72.0913 | | | | |
| mL) | | | | | |
| tₘₐₓ (hr) | 10.53 ± | 52.94 | 8.06 | 2.00 | 24.00 |
| | 5.58 | | | | |

TABLE 31
Geometric Mean of Test Product (T) and Reference product (R) of
Estradiol—Baseline adjusted (N = 34)

| | Geometric Mean | |
|---|---|---|
| Pharmacokinetic | Test | Reference |
| Parameter | Product (T) | Product (R) |
| Cₘₐₓ (pg/mL) | 14.3774 | 20.3837 |
| AUC₀₋₂₄ (pg · hr/mL) | 49.6231 | 132.9218 |
| tₘₐₓ (hr) | 1.75 | 9.28 |

TABLE 32
Statistical Results of Test product (T) versus Reference product (R) for
Estradiol—Baseline adjusted (N = 34)

| | Geometric Least | | | | |
|---|---|---|---|---|---|
| | Square Mean | | | | |
| Pharma- | Test | Reference | Intra | | 90% |
| cokinetic | Product | Product | Subject | T/R | Confidence |
| Parameter | (T) | (R) | CV % | Ratio % | Interval |
| Cₘₐₓ (pg/mL) | 14.4490 | 20.1980 | 60.68 | 71.54* | 56.82-90.08 |
| AUC₀₋₂₄ | 49.7310 | 131.0400 | 70.64 | 37.95* | 29.21-49.31 |
| (pg · hr/mL) | | | | | |

*Comparison was detected as statistically significant by ANOVA (α = 0.05).

The PK data for total estrone likewise demonstrated reduced systemic exposure when compared to the RLD. Table 33 shows the pharmaceutical compositions disclosed herein reduced systemic exposure by 2500 for Cₘₐₓ and 49% for AUC.

TABLE 33
Summary of Pharmacokinetic Parameters of Test product (T) of
Estrone—Baseline adjusted (N = 33)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 6.8485 ± | 96.1149 | 5.4000 | 1.3000 | 36.3000 |
| (pg/mL) | 6.5824 | | | | |
| AUC₀₋₂₄ | 34.7051 ± | 80.5476 | 30.8500 | 3.3500 | 116.7500 |
| (pg · hr/ | 27.9541 | | | | |
| mL) | | | | | |
| tₘₐₓ (hr) | 9.12 ± | 96.80 | 4.00 | 1.00 | 24.00 |
| | 8.83 | | | | |

TABLE 34
Summary of Pharmacokinetic Parameters of Reference product (R) of
Estrone—Baseline adjusted (N = 33)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 8.8333 ± | 80.9086 | 6.7000 | 2.7000 | 30.3000 |
| (pg/mL) | 7.1469 | | | | |
| AUC₀₋₂₄ | 63.0042 ± | 73.8814 | 51.2800 | 8.8000 | 214.0000 |
| (pg · hr/ | 46.5484 | | | | |
| mL) | | | | | |
| tₘₐₓ (hr) | 11.16 ± | 64.95 | 10.00 | 4.00 | 24.00 |
| | 7.24 | | | | |

TABLE 35
Geometric Mean of Test Product (T) and Reference product (R) of
Estrone—Baseline adjusted (N = 33)

| | Geometric Mean | |
|---|---|---|
| Pharmacokinetic | Test | Reference |
| Parameter | Product (T) | Product (R) |
| Cₘₐₓ (pg/mL) | 5.1507 | 6.9773 |
| AUC₀₋₂₄ (pg · hr/mL) | 24.2426 | 48.2377 |
| tₘₐₓ (hr) | 5.87 | 9.07 |

TABLE 36
Statistical Results of Test product (T) versus Reference product (R) for
Estrone—Baseline adjusted (N = 33)

| | Geometric Least | | | | |
|---|---|---|---|---|---|
| | Square Mean | | | | |
| Pharma- | Test | Reference | Intra | | 90% |
| cokinetic | Product | Product | Subject | T/R | Confidence |
| Parameter | (T) | (R) | CV % | Ratio % | Interval |
| Cₘₐₓ (pg/mL) | 5.1620 | 6.9280 | 47.59 | 74.50* | 61.69-89.97 |
| AUC₀₋₂₄ | 24.1960 | 47.9020 | 73.66 | 50.51* | 38.37-66.50 |
| (pg · hr/mL) | | | | | |

*Comparison was detected as statistically significant by ANOVA (α = 0.05).

The PK data for estrone sulfate likewise demonstrated reduced systemic exposure when compared to the RLD. Table 37 shows the pharmaceutical compositions disclosed herein reduced systemic exposure by 25% for Cₘₐₓ and 42% for AUC.

TABLE 37
Summary of Pharmacokinetic Parameters of Test product (T) of Estrone
Sulfate—Baseline adjusted (N = 24)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 13.9042 ± | 50.6339 | 11.1500 | 1.3000 | 39.0000 |
| (ng/mL) | 7.0402 | | | | |
| AUC₀₋₂₄ | 97.9953 ± | 82.5408 | 76.2750 | 5.1025 | 338.0000 |
| (ng · hr/ | 80.8861 | | | | |
| mL) | | | | | |
| tₘₐₓ (hr) | 6.33 ± | 71.93 | 4.00 | 4.00 | 24.00 |
| | 4.56 | | | | |

TABLE 38
Summary of Pharmacokinetic Parameters of Reference product (R) of
Estrone Sulfate—Baseline adjusted (N = 24)

| | Arithmetic | | | | |
|---|---|---|---|---|---|
| Pharma- | Mean ± | Coefficient | | | |
| cokinetic | Standard | of | | | |
| Parameter | Deviation | Variation | Median | Minimum | Maximum |
| Cₘₐₓ | 19.2542 ± | 59.0173 | 15.2000 | 7.0000 | 53.7000 |
| (ng/mL) | 11.3633 | | | | |
| AUC₀₋₂₄ | 177.6208 ± | 93.5931 | 124.0000 | 20.0000 | 683.0500 |
| (ng · hr/ | 166.2408 | | | | |
| mL) | | | | | |
| tₘₐₓ (hr) | 10.33 ± | 54.05 | 10.00 | 2.00 | 24.00 |
| | 5.58 | | | | |

TABLE 39
Geometric Mean of Test Product (T) and Reference product (R) of Estrone
Sulfate—Baseline adjusted (N = 24)

| | Geometric Mean | |
|---|---|---|
| Pharmacokinetic | Test | Reference |
| Parameter | Product (T) | Product (R) |
| Cₘₐₓ (ng/mL) | 12.1579 | 16.8587 |
| AUC₀₋₂₄ (ng · hr/mL) | 66.5996 | 121.5597 |
| tₘₐₓ (hr) | 5.49 | 8.83 |

TABLE 40
Statistical Results of Test product (T) versus Reference product (R) for
Estrone Sulfate—Baseline adjusted (N = 24)

| | Geometric Least | | | | |
|---|---|---|---|---|---|
| | Square Mean | | | | |
| Pharma- | Test | Reference | Intra | | 90% |
| cokinetic | Product | Product | Subject | T/R | Confidence |
| Parameter | (T) | (R) | CV % | Ratio % | Interval |
| Cₘₐₓ (ng/mL) | 12.3350 | 16.5470 | 48.02 | 74.55* | 59.43-93.51 |
| AUC₀₋₂₄ | 68.5260 | 118.4170 | 73.87 | 57.87* | 41.68-80.35 |
| (ng · hr/mL) | | | | | |

*Comparison was detected as statistically significant by ANOVA (α = 0.05).

### Example 10: Randomized, Double-Blind, Placebo-Controlled Multicenter Study of Estradiol Vaginal Softgel Capsules for Treatment of VVA

### Investigational Plan

The study was a randomized, double-blind, placebo-controlled multicenter study design. Postmenopausal subjects who meet the study entry criteria will be randomized in a 1:1:1:1 ratio to receive Estradiol Vaginal Softgel Capsule 4 μg, Estradiol Vaginal Softgel Capsule 10 μg, Estradiol Vaginal Softgel Capsule 25 μg, or matching placebo. Subjects will be asked to self-assess the symptoms of vulvar or vaginal atrophy including vaginal pain associated with sexual activity, vaginal dryness, vulvar or vaginal itching or irritation by completing the VVA symptom self-assessment questionnaire and identification of her MBS at screening visit 1A to determine eligibility for the study. The VVA symptom Self-Assessment Questionnaire, vaginal cytology, vaginal pH, and vaginal mucosa will be assessed at screening visit 1B. These assessments will determine continued eligibility and will be used as the baseline assessments for the study. Randomized subjects will then complete the Questionnaire during visits 3, 4, 5, and 6.

The primary efficacy endpoints for the study included: (A) change from baseline to week 12 in the percentage of vaginal superficial cells (by vaginal cytologic smear) compared to placebo; (B) change from baseline to week 12 in the percentage of vaginal parabasal cells (by vaginal cytologic smear) compared to placebo; (C) change from baseline at week 12 in vaginal pH as compared to placebo; and (D) change from baseline to week 12 on the severity of the MBS of dyspareunia (vaginal pain associated with sexual activity) associated with VVA as compared to placebo.

The secondary efficacy endpoints for the study included: (E) change from baseline to weeks 2, 6, and 8 in the percentage of vaginal superficial cells (by vaginal cytologic smear) compared to placebo; (F) change from baseline to weeks 2, 6, and 8 in the percentage of vaginal parabasal cells (by vaginal cytologic smear) compared to placebo; (G) change from baseline to weeks 2, 6, and 8 in vaginal pH as compared to placebo; (H) change from baseline to weeks 2, 6, and 8 on the severity of the MBS of dyspareunia (vaginal pain associated with sexual activity) associated with VVA as compared to placebo; (I) change from baseline to weeks 2, 6, 8, and 12 on the severity of vaginal dryness and vulvar or vaginal itching or irritation associated with VVA as compared to placebo; (J) change in visual evaluation of the vaginal mucosa from baseline to weeks 2, 6, 8, and 12 compared to placebo; (K) assessment of standard PK parameters as defined in the SAP for serum estradiol, estrone, and estrone conjugates at Screening Visit 1A, days 1, 14, and 84 of treatment in a subset of subjects (PK substudy) utilizing baseline corrected and uncorrected values [as outlined in the Statistical Analysis Plan (SAP)]; and (L) change from baseline in the Female Sexual Function Index (FSFI) at week 12 compared to placebo.

The safety endpoints for the study included: (1) Adverse events; (2) Vital signs; (3) Physical examination findings; (4) Gynecological examination findings; (5) Clinical laboratory tests; (6) Pap smears; and (7) Endometrial biopsy.

Approximately 100 sites in the United States and Canada screened approximately 1500 subjects to randomize 747 subjects in this study (modified intent to treatment population, or all subjects who have taken at least one dose of the pharmaceutical compositions disclosed herein), with a target of 175 subjects randomized to each treatment group (175 in each active treatment group and 175 in the placebo group to complete 560 subjects). Actual subjects enrolled are 186 subjects in the 4 μg formulation group, 188 subjects in the 10 μg formulation group, 186 subjects in the 25 μg formulation group, and 187 subjects in the placebo group, for a total of 747 subjects in the study. Within each treatment group, 15 subjects also participated in a PK substudy. Subjects were assigned to one of four treatment groups: (1) 4 μg formulation; (2) 10 μg formulation; (3) 25 μg formulation; and (4) placebo.

Most subjects participated in the study for 20-22 weeks. This included a 6 to 8 week screening period (6 weeks for subjects without an intact uterus and 8 weeks for subjects with an intact uterus), 12 weeks on the investigational product, and a follow-up period of approximately 15 days after the last dose of investigational product. Some subjects' involvement lasted up to 30 weeks when an 8-week wash-out period was necessary. Subjects who withdrew from the study were not replaced regardless of the reason for withdrawal.

The study schematic diagram shown in FIG. 9. There were two treatment periods; once daily intravaginal administration of one of the listed investigational products for 2 weeks, followed by a twice weekly intravaginal administration for 10 weeks.

The subject inclusion criteria included: (1) postmenopausal female subjects between the ages of 40 and 75 years (at the time of randomization) with at least: 12 months of spontaneous amenorrhea (women <55 years of age with history of hysterectomy without bilateral oophorectomy prior to natural menopause must have follicle stimulating hormone (FSH) levels >40 mIU/mL); or 6 months of spontaneous amenorrhea with follicle stimulating hormone (FSH) levels >40 mlU/mL; or At least 6 weeks postsurgical bilateral oophorectomy.

The subject inclusion criteria also included: (2) ≤5% superficial cells on vaginal cytological smear; (3) Vaginal pH >5.0; (4) Moderate to severe symptom of vaginal pain associated with sexual activity considered the most bothersome vaginal symptom by the subject at screening visit 1A; (5) Moderate to severe symptom of vaginal pain associated with sexual activity at screening visit 1B; (6) Onset of moderate to severe dyspareunia in the postmenopausal years; (7) Subjects were sexually active (i.e., had sexual activity with vaginal penetration within approximately 1 month of screening visit 1A); and (8) Subjects anticipated having sexual activity (with vaginal penetration) during the conduct of the trial

For subjects with an intact uterus, the subject inclusion criteria also included: (9) subjects had an acceptable result from an evaluable screening endometrial biopsy. The endometrial biopsy reports by the two central pathologists at screening specified one of the following: proliferative endometrium; weakly proliferative endometrium; disordered proliferative pattern; secretory endometrium; endometrial tissue other (i.e., benign, inactive, or atrophic fragments of endometrial epithelium, glands, stroma, etc.); endometrial tissue insufficient for diagnosis; no endometrium identified; no tissue identified; endometrial hyperplasia; endometrial malignancy; or other findings (endometrial polyp not present, benign endometrial polyp, or other endometrial polyp). Identification of sufficient tissue to evaluate the biopsy by at least one pathologist was required.

For subjects with a Body Mass Index (BMI) less than or equal to 38 kg/m², the subject inclusion criteria also included: (10) BMI values were rounded to the nearest integer (ex. 32.4 rounds down to 32, while 26.5 rounds up to 27).

In general, the inclusion criteria also included: (11) in the opinion of the investigator, the subject was believed likely to comply with the protocol and complete the study.

The exclusion criteria included: (1) use of oral estrogen-, progestin-, androgen-, or SERM-containing drug products within 8 weeks before screening visit 1A (entry of washout was permitted); use of transdermal hormone products within 4 weeks before screening visit 1A (entry of washout was permitted); use of vaginal hormone products (rings, creams, gels) within 4 weeks before screening visit 1A (entry of washout was permitted); use of intrauterine progestins within 8 weeks before screening visit 1A (entry of washout was permitted); use of progestin implants/injectables or estrogen pellets/injectables within 6 months before screening visit 1A (entry of washout was not permitted); or use of vaginal lubricants or moisturizers within 7 days before the screening visit 1B vaginal pH assessment.

The exclusion criteria also included: (2) a history or active presence of clinically important medical disease that might confound the study or be detrimental to the subject, including, for example: hypersensitivity to estrogens; endometrial hyperplasia; undiagnosed vaginal bleeding; a history of a chronic liver or kidney dysfunction/disorder (e.g., Hepatitis C or chronic renal failure); thrombophlebitis, thrombosis, or thromboembolic disorders; cerebrovascular accident, stroke, or transient ischemic attack; myocardial infarction or ischemic heart disease; malignancy or treatment for malignancy, within the previous 5 years, with the exception of basal cell carcinoma of the skin or squamous cell carcinoma of the skin (a history of estrogen dependent neoplasia, breast cancer, melanoma, or any gynecologic cancer, at any time, excluded the subject); and endocrine disease (except for controlled hypothyroidism or controlled non-insulin dependent diabetes mellitus).

The exclusion criteria also included: (3) recent history of known alcohol or drug abuse; (4) history of sexual abuse or spousal abuse that was likely to interfere with the subject's assessment of vaginal pain with sexual activity; (5) current history of heavy smoking (more than 15 cigarettes per day) or use of e-cigarettes; (6) use of an intrauterine device within 12 weeks before screening visit 1A; (7) use of an investigational drug within 60 days before screening visit 1A; (8) any clinically important abnormalities on screening physical exam, assessments, electrocardiogram (ECG), or laboratory tests; (9) known pregnancy or a positive urine pregnancy test; and (10) current use of marijuana.

In this study, if a subject discontinued or was withdrawn, the subject was not replaced. At the time of consent, each subject was given a unique subject number that identified their clinical site and sequential number. In addition to the assigned subject number, subject initials were used for identification. The clinical trial was performed in compliance with standard operating procedures as well as regulations set forth by FDA, ICH E6 (R1) guidelines, and other relevant regulatory authorities. Compliance was achieved through clinical trial-specific audits of clinical sites and database review.

### Statistical Methods

Efficacy. The primary objective of the trial was to assess the efficacy of estradiol vaginal softgel capsules (4 μg, 10 μg, and 25 μg) when compared to placebo on vaginal superficial cells, vaginal parabasal cells, vaginal pH, and on the symptom of moderate to severe dyspareunia (vaginal pain associated with sexual activity) as the MBS at week 12. To account for the multiple comparisons of testing placebo to each of the three doses of estradiol (4 μg, 10 μg, and 25 μg) and the multiple testing of the four co-primary endpoints, a closed procedure was performed (see, Edwards D, Madsen J. “Constructing multiple procedures for partially ordered hypothesis sets.” Stat Med 2007:26-5116-24, incorporated by reference herein).

Determination of Sample Size. The sample size needed per dose vs. placebo for each test of hypothesis in the modified intent-to-treat (MITT) population to achieve a given power was calculated using reference data from other studies (see, Bachman, G., et al. “Efficacy and safety of low-dose regimens of conjugated estrogens cream administered vaginally.” Menopause, 2009. 16(4): p. 719-27; Simon, J., et al. “Effective Treatment of Vaginal Atrophy With an Ultra-Low-Dose Estradiol Vaginal Tablet.” Obstetrics & Gynecology, 2008. 112(5):p. 1053-60; FDA Medical Officer's Review of Vagifem [NDA 20-908, Mar. 25, 1999, Table 6, p 12.], each incorporated by reference herein). Table 41 below provides the effect sizes, power, and sample size determinations for each of the primary endpoints. In general, subjects in the study met all inclusion/exclusion criteria and had moderate to severe dyspareunia as their most bothersome symptom of VVA. Based on the power analysis and the design considerations, approximately 175 subjects per treatment arm were enrolled.

TABLE 41
Power Analysis and Sample Size Determinations

| | | Power Based Upon |
|---|---|---|
| Primary Endpoint | Effect Size (%)* | N = 140 per group per MITT |
| % Parabasal Cells | 150.3% | >0.999 |
| % Superficial Cells | 115.3% | >0.999 |
| Vaginal pH | 77.4% | >0.999 |
| Severity of Dyspareunia** | 30.0%, 41.2%, 70.5% | 0.50, 0.80, >0.999 |

*Range from 30% (Vagifem 10 μg; see, Simon 2008, supra), 41.2% (Vagifem 25 μg; see FDA 1999, supra),70.5% (Premarin cream 2/week; see, Bachman 2009, supra)
**Effect Size is calculated for all primary endpoints as 100% times difference (treated minus placebo) in mean changes at week 12 from baseline.
Four Primary Endpoints in a Closed Procedure
Mean Change from Baseline to Week 12 Compared to Placebo (MMRM)
Power (One-way ANOVA, Alpha = 0.005, one-tailed)

All subjects who were randomly assigned and had at least 1 dose of investigational product formed the intent-to-treat (ITT) population. The Modified intent-to-treat (MITT) population was defined as all ITT subjects with a baseline and at least one follow-up value for each of the primary endpoints, each subject having taken at least one dose of investigational product, and was the primary efficacy population. The efficacy-evaluable (EE) population was defined as all MITT subjects who completed the clinical trial, were at least 80% compliant with investigational product, had measurements for all primary efficacy endpoints, and were deemed to be protocol compliant, with no significant protocol violations. The safety population included all ITT subjects.

The primary efficacy analyses were conducted on the MITT subjects with supportive efficacy analyses conducted on the EE population. For analysis purposes, subjects were required to complete all visits, up to and including Visit 6 (week 12), to be considered as having completed the study.

Analysis of Efficacy Endpoints. For all numerically continuous efficacy endpoints, which included the four primary endpoints (mean change from baseline to week 12), active treatment group means were compared to placebo using an ANCOVA adjusting for the baseline level.

Primary and secondary efficacy endpoints were measured at baseline and at 2, 6, 8, and 12 weeks. The analysis examined change from baseline. Therefore, ANCOVAs were based on a repeated measures mixed effects model (MMRM) where the random effect was subject and the two fixed effects were treatment group and visit (2, 6, 8, and 12 weeks). Baseline measures and age were used as covariates. ANCOVAs were therefore not calculated independently for each study collection period. The analyses started with the full model but, interaction terms for visit (week 2, 6, 8, and 12) with treatment only remained where statistically significant (p<0.05).

The following three pair-wise comparisons were performed using the appropriate ANCOVA contrast for week 12 (primary) and weeks 2, 6, and 8 (secondary) changes from baseline: (1) active treatment, high dose group vs placebo; (2) active treatment, middle dose group vs placebo; and (3) active treatment, low dose group vs placebo.

Safety outcome measures. Adverse events, vital signs, physical examination findings, gynecological examination findings, clinical laboratory tests, pap smears, and endometrial biopsy were the safety parameters. Adverse events and SAEs were summarized for each treatment group and overall for all active treatment groups with the proportion of subjects reporting each event. Actual values and change from baseline in vital signs, and all laboratory test parameters were summarized for each treatment group and overall for all active treatment groups with descriptive statistics at each assessment obtained.

Endometrial Biopsy Assessment. Three independent pathologists with expertise in gynecologic pathology, blinded to treatment and to each other's readings, determined the diagnosis for endometrial biopsy slides during the conduct of the study. All visit 6, early termination, and on-treatment unscheduled endometrial biopsies were centrally read by three of the pathologists. Each pathologist's report was classified into one of the following three categories: category 1: not hyperplasia/not malignancy—includes proliferative endometrium, weakly proliferative endometrium, disordered proliferative pattern, secretory endometrium, endometrial tissue other (i.e., benign, inactive or atrophic fragments of endometrial epithelium, glands, stroma, etc.), endometrial tissue insufficient for diagnosis, no endometrium identified, no tissue identified, other; category 2: hyperplasia—includes simple hyperplasia with or without atypia and complex hyperplasia with or without atypia; category 3: malignancy—endometrial malignancy.

The final diagnosis was based on agreement of two of the three reads. Consensus was reached when two of the three pathologist readers agreed on any of the above categories. For example, any 2 subcategories of “not hyperplasia/not malignancy” were classified as “Category 1: not hyperplasia/not malignancy.” If all three readings were disparate (i.e., each fell into a different category—category 1, 2, or 3), the final diagnosis was based on the most severe of the three readings.

The analysis population for endometrium hyperplasia was the endometrial hyperplasia (EH) population. An EH subject at week 12 was one who was randomly assigned and took at least 1 dose of investigational product, with no exclusionary protocol violation (as detailed at the Statistical Analysis Plan), and had a pretreatment endometrial biopsy and a biopsy on therapy.

### Treatment of Subjects

The study used a double-blind design. Investigational product was supplied as 3 doses of Estradiol Vaginal Softgel Capsules (4 μg, 10 μg, and 25 μg) and matching placebo capsules. All subjects manually inserted one capsule into the vaginal cavity daily for 14 days (2 weeks) followed by twice weekly for 10 weeks according to one of the following treatment arms:

TABLE 42
Treatment Arms and Administration

| Regimen | Capsules | Capsules |
|---|---|---|
| Treatment | 1 capsule daily of | 1 capsule twice weekly of |
| 1 | 4 μg vaginal softgel | 4 μg vaginal softgel for |
| | for 2 weeks | 10 weeks |
| Treatment | 1 capsule daily of | 1 capsule twice weekly of |
| 2 | 10 μg vaginal softgel | 10 μg vaginal softgel for |
| | for 2 weeks | 10 weeks |
| Treatment | 1 capsule daily of | 1 capsule twice weekly of |
| 3 | 25 μg vaginal softgel | 25 μg vaginal softgel for |
| | for 2 weeks | 10 weeks |
| Treatment | 1 capsule daily of | 1 capsule twice weekly of |
| 4 | placebo vaginal | placebo vaginal softgel |
| | softgel for 2 weeks | for 10 weeks |

Investigational product was dispensed to all eligible subjects at visit 2. Each subject was provided a total of 30 soft gel capsules of investigational product in a labeled bottle, allowing for extra capsules for accidental loss or damage. A second bottle was dispensed at Visit 5. Each subject was trained by the clinical site to self-administer intravaginally one capsule daily at approximately the same hour for 2 weeks (14 days). The drug administration instructions included: “Remove vaginal capsule from the bottle; find a position most comfortable for you; insert the capsule with the smaller end up into vaginal canal for about 2 inches.” Starting on Day 15, each subject administered 1 capsule twice weekly for the remaining 10 weeks. Twice weekly dosing was approximately 3-4 days apart, and generally did not exceed more than twice in a seven day period. For example, if the Day 15 dose was inserted on Sunday, the next dose was inserted on Wednesday or Thursday. At randomization visit 2 (day 1), subjects received their first dose of investigational product at the clinical facility under the supervision of the study personnel.

The investigational estradiol vaginal softgel drug products used in the study are pear-shaped, opaque, light pink softgel capsules. The capsules contain the solubilized estradiol pharmaceutical compositions disclosed herein as Pharmaceutical Compositions 4-7. When the softgel capsules come in contact with the vaginal mucosa, the soft gelatin capsule releases the pharmaceutical composition, into the vagina. In embodiments, the soft gelatin capsule completely dissolves.

The placebo used in the study contained the excipients in the investigational estradiol vaginal softgel capsule without the estradiol (see, e.g., Pharmaceutical Composition 7). The packaging of the investigational products and placebo were identical to maintain adequate blinding of investigators. Neither the subject nor the investigator was able to identify the treatment from the packaging or label of the investigational products.

A subject was required to use at least 80% of the investigational product to be considered compliant with investigational medication administration. Capsule count and diary cards were be used to determine subject compliance at each study visit. Subjects were randomly assigned in a 1:1:1:1 ratio to receive Estradiol Vaginal Softgel Capsule 4 μg (Pharmaceutical Composition 4), Estradiol Vaginal Softgel Capsule 10 μg (Pharmaceutical Composition 5), Estradiol Vaginal Softgel Capsule 25 μg (Pharmaceutical Composition 6), or placebo (Pharmaceutical Composition 7).

Concomitant medications/treatments were used to treat chronic or intercurrent medical conditions at the discretion of the investigator. The following medications were prohibited for the duration of the study: investigational drugs other than the investigational Estradiol Vaginal Softgel Capsule; estrogen-, progestin-, androgen (i.e., DHEA) or SERM-containing medications other than the investigational product; medications, remedies, and supplements known to treat vulvar/vaginal atrophy; vaginal lubricants and moisturizers (e.g., Replens) be discontinued 7 days prior to Visit 1B vaginal pH assessment; and all medications excluded before the study,

### Efficacy Assessments

Vaginal cytological smears were collected from the lateral vaginal walls according to standard procedures and sent to a central laboratory to evaluate vaginal cytology. The percentage of superficial, parabasal, and intermediate cells was determined. All on-therapy/early termination vaginal cytology results were blinded to the Sponsor, Investigators, and subjects.

Vaginal pH was determined at screening Visit 1B and visits 3, 4, 5, and 6/end of treatment. Subjects were not allowed to use vaginal lubricants or moisturizers within 7 days of the screening vaginal pH assessment or at any time afterwards during the study. The subjects were advised not to have sexual intercourse and to refrain from using vaginal douching within 24 hours prior to the measurement for all scheduled vaginal pH assessments. After insertion of an unlubricated speculum, a pH indicator strip was applied to the lateral vaginal wall until it became wet, taking care to avoid cervical mucus, blood or semen that are known to affect vaginal pH. The color of the strip was compared immediately with a colorimetric scale and the measurement was recorded.

During the gynecological examinations, the investigator performed a visual evaluation of vaginal mucosa using a four-point scale (0=none, 1=mild, 2=moderate, and 3=severe) to assess parameters of vaginal secretions, vaginal epithelial integrity, vaginal epithelial surface thickness, and vaginal color according to the table below.

| Assessment | Severity | | | |
|---|---|---|---|---|
| Criteria | No atrophy | Mild | Moderate | Severe |
| Vaginal | normal clear | superficial coating | scant not covering | none, inflamed, |
| secretions | secretions | of secretions, | the entire vaginal | ulceration noted, |
| | noted on | difficulty with | vault, may need | need lubrication |
| | vaginal walls | speculum insertion | lubrication with | with speculum |
| | | | speculum insertion | insertion to prevent |
| | | | to prevent pain | pain |
| Vaginal | normal | vaginal surface | vaginal surface | vaginal surface has |
| epithelial | | bleeds with | bleeds with light | petechiae before |
| integrity | | scraping | contact | contact and bleeds |
| | | | | with light contact |
| Vaginal | rogation and | poor rogation with | smooth, some | smooth, no elasticity, |
| epithelial | elasticity of | some elasticity | elasticity of | constriction of the |
| surface | vault | noted of vaginal | vaginal vault | upper one third of |
| thickness | | vault | | vagina or loss of |
| | | | | vaginal tone |
| | | | | (cystocele and |
| | | | | rectocele) |
| Vaginal | pink | lighter in color | pale in color | transparent, either no |
| color | | | | color or inflamed |

The VVA symptom self-assessment questionnaire, shown below, is an instrument for subjects to self-assess their symptoms of vulvar or vaginal atrophy, including vaginal pain associated with sexual activity, vaginal dryness, vulvar or vaginal itching, or irritation. At screening visit 1A subjects were asked to complete the questionnaire and identify their most bothersome symptoms, and the results of the survey were used to determine initial eligibility for the study. At visit 1A, subjects were also asked to indicate which moderate or severe symptoms bothered them most. The questionnaire was administered again at screening visit 1B and used to determine continued eligibility for the study.

VVA SYMPTOMS SELF-ASSESSMENT

| Please Rate | | Severity Score | | | |
|---|---|---|---|---|---|
| your Vulvar and/or | | (Please select only ONE) | | | |
| Vaginal Symptoms | | 0 = None | 1 = mild | 2 = Moderate | 3 = Severe |
| 1 | Pain associated | | | | |
| | with sexual | | | | |
| | activity (with | | | | |
| | vaginal | | | | |
| | penetration). | | | | |
| 2 | Vaginal dryness. | | | | |
| 3 | Vulvar and/or | | | | |
| | vaginal itching | | | | |
| | or irritation. | | | | |

Randomized subjects were asked to complete the VVA Symptom Self-Assessment Questionnaire at visits 3, 4, 5, and 6. Subjects were asked to indicate if no sexual activity with vaginal penetration was experience since the previous visit. Screening visit 1B evaluation results were considered as Baseline data for the statistical analyses.

The Female Sexual Function Index (FSFI) is a brief, multidimensional scale for assessing sexual function in women (see, Rosen, 2000, supra 26: p. 191-208, incorporated by reference herein). The scale consists of 19 items that assess sexual function over the past 4 weeks and yield domain scores in six areas: sexual desire, arousal, lubrication, orgasm, satisfaction, and pain. Further validation of the instrument was conducted to extend the validation to include dyspareunia/vaginismus (pain), and multiple sexual dysfunctions (see, Weigel, M., et al. “The Female Sexual Function Index (FSFI): Cross-Validation and Development of Clinical Cutoff Scores.” Journal of Sex & Marital Therapy, 2005. 31: p. 1-20, incorporated by reference herein). The FSFI was conducted at Visits 2 and 6. Subjects participating in the PK substudy were not assessed using FSFI.

### Safety Assessments

A complete medical history, including demographic data (age and race/ethnicity) gynecological, surgical, and psychiatric history and use of tobacco and alcohol was recorded at the washout/screening visit 1A prior to any washout period; this history included a review of all past and current diseases and their respective durations as well as any history of amenorrhea.

A complete physical examination was conducted at screening visit 1A and visit 6/end of treatment. The physical examination included, at a minimum, examination of the subject's general appearance, HEENT (head, eyes, ears, nose, and throat), heart, lungs, musculoskeletal system, gastrointestinal (GI) system, neurological system, lymph nodes, abdomen, and extremities. The subject's height was measured at washout/screening visit 1A only and body weight (while the subject is lightly clothed) was be measured at washout/screening visit 1A and end of treatment. BMI was calculated at washout/screening visit 1A. Vital signs (body temperature, heart rate [HR], respiration rate [RR], and sitting blood pressure [BP]) were measured at all visits after the subject had been sitting for ≥10 minutes. If the initial BP reading was above 140 mmHg systolic or 90 mmHg diastolic, the option for a single repeat assessment performed 15 minutes later was provided. A standard 12-lead ECG was obtained at screening visit 1A and visit 6 or early termination.

Subjects were required to have a pelvic examination and Pap smear performed during the screening visit 1B and visit 6 or early termination. The Pap smear was required for all subjects with or without an intact uterus and cervix. For subjects without an intact cervix the Pap smear was obtained by sampling the apex of the vaginal cuff. All subjects were required to have a Pap smear done during screening, regardless of any recent prior assessment. Subjects who discontinued the study after 2 weeks of investigational product were required to have an end of treatment Pap smear. Subjects had a breast examination performed during screening visit 1A and at visit 6 or early termination.

Endometrial biopsies were performed by a board-certified gynecologist at screening and at visit 6/end of treatment. Unscheduled endometrial biopsies were performed during the study, when indicated for medical reasons. The screening biopsy was performed at screening visit 1B, after the subject's initial screening visit assessments indicated that the subject was otherwise an eligible candidate for the study.

At screening, endometrial biopsies were read centrally by two pathologists. A candidate subject was excluded from the study if at least one pathologist assessed the endometrial biopsy as endometrial hyperplasia, endometrial cancer, proliferative endometrium, weakly proliferative endometrium, or disordered proliferative pattern, or if at least one pathologist identified an endometrial polyp with hyperplasia, glandular atypia of any degree (e.g., atypical nuclei), or cancer. Additionally, identification of sufficient tissue to evaluate the biopsy by at least one pathologist was required for study eligibility. The option for one repetition of the screening endometrial biopsy was made available when an initial endometrial biopsy was performed and both of the primary pathologists reported endometrial tissue insufficient for diagnosis, no endometrium was identified, or no tissue was identified, and if the subject had met all other protocol-specified eligibility criteria to date. The visit 6 (or early termination) endometrial biopsies and on treatment unscheduled biopsies were assessed by three pathologists.

During the study, at early termination, and at the end of the study, any subject with a diagnosis of endometrial hyperplasia was withdrawn and treated with 10 mg of Medroxyprogesterone acetate (MPA) for 6 months unless deemed otherwise by the PI. For unscheduled biopsies, the histological diagnosis of endometrial polyp did not force withdrawal unless atypical nuclei were present.

A urine pregnancy test was conducted at screening visit 1A unless the subject had a history of tubal ligation, bilateral oophorectomy, or was ≥55 years of age and had experienced cessation of menses for at least 1 year.

Blood samples for blood chemistry, hematology, coagulation tests, and hormone levels and urine samples for urine analysis were collected and sent to a central laboratory. Blood Chemistry (sodium, potassium, chloride, total cholesterol, blood urea nitrogen (BUN), iron, albumin, total protein, aspartate aminotransferase (AST), alanine aminotransferase (ALT), alkaline phosphatase, creatinine, calcium, phosphorous, uric acid, total bilirubin, glucose and triglycerides (must be fasting minimum of 8 hours). A fasting glucose of >125 mg/dL will require a HgA1C) was monitored. Hematology (complete blood count (CBC) including white blood cell count and differential, red blood cell count, hemoglobin, hematocrit, and platelet count) was monitored. Hormone Levels (follicle-stimulating hormone (FSH) (not required for subjects with ≥12 months of spontaneous amenorrhea or bilateral oophorectomy), estradiol, estrone, and estrone conjugates and SHBG for subjects in the PK substudy) were monitored. Urine Analysis (appearance, specific gravity, protein, and pH) was conducted.

### Pharmacokinetic Assessment

Seventy-two subjects were also enrolled in a pharmacokinetic (PK) substudy. In those subjects participating in the PK substudy, time 0 h serum blood samples were obtained at screening visit 1A, day 1, and day 14 prior to dosing for baseline. The baseline was characterized by the average of the two pre-treatment samples. Serum blood samples were then obtained on day 1 and day 14 at five post dose time points (2 h, 4 h, 6 h, 10 h, and 24 h). On study days 1 (visit 2) and 14 (visit 3) a baseline pretreatment blood sample (Time 0 h) was collected from each subject prior to insertion of the investigational product. After insertion of the product, blood samples were drawn at 2, 4, 6, 10, and 24 hours following insertion. The last PK sample (approximately day 84) was obtained 4 days following the last insertion of investigational product.

Blood samples were analyzed to characterize area under the curve (AUC), time of maximum concentration (tₘₐₓ), minimum concentration (Cₘᵢₙ), and maximum concentration (Cₘₐₓ). Blood samples were also analyzed to measure the levels of estradiol, estrone, and estrone conjugates. No fasting requirements were applied. Sex hormone binding globulin (SHBG) levels were obtained at pre-treatment baseline (day 1, visit 2), and day 14 at the 0 h and on the day 84 final hormone blood draw.

A symptoms/complaints and medications diary was dispensed at all visits and subjects were instructed on completion. The subjects used the diary to record symptoms/complaints (including stop and start dates and treatment received) and prior medications/treatments (including indication, stop, and start dates). A copy of the diary was made at each visit and re-dispensed to the subject. A dosing diary was dispensed at visit 2 and at visit 3 and subjects were be instructed on completion. Subjects recorded investigational product usage and sexual activity. The dosing diary dispensed at visit 3 was re-dispensed at visits 4 and 5. A copy of the diary was made at each visit prior to re-dispensing to the subject.

### Study Visits

Study visits were typically conducted so as to include the activities outlined in Table 43.

TABLE 43
Schedule of Assessments-Main Study

| | | | | Visit 2: | |
|---|---|---|---|---|---|
| | | Visit 1A | Visit 1B | Randomization/ | Visit 3: |
| | Washout | Screening | Screening | Baseline | Interim |
| | | | | | Week 2 |
| | Week −14 to | Week −6 to | Week −4 to | Week 0 | Day 14 |
| Activity | −6 | 0 | 0 | Day 1 | (±3d) |
| Informed consent | X | X | | | |
| Demographics/Medical and | X | X | | | |
| Gynecological history and | | | | | |
| prior medications | | | | | |
| Weight | X | X | | | |
| Height and BMI calculation | X | X | | | |
| Vital signs | X | X | X | X | X |
| MBS | | X | | | |
| Subject VVA Self-Assessment | | X | X | | X |
| Questionnaire | | | | | |
| Physical examination including | | X | | | |
| breast exam | | | | | |
| Laboratory safety tests | | X | | | |
| (Hematology, Serum Chemistry, | | | | | |
| FSHP, Urinalysis) | | | | | |
| 12-Lead ECG | | X | | | |
| Pelvic exam | | | X | | |
| Vaginal pH | | | X | | X |
| Papanicolaou (Pap) smear | | | X | | |
| Investigator assessment of vaginal | | | X | | X |
| mucosa | | | | | |
| Vaginal cytological smear | | | X | | X |
| Mammogram | | X | | | |
| Endometrial biopsy | | | X | | |
| Diary Dispense | X | X | X | X | X |
| Diary Collection | | X | X | X | X |
| FSFI | | | | X | |
| Satisfaction Survey | | | | | |
| Urine pregnancy test | | X | | | |
| Randomization | | | | X | |
| Dispense Investigational | | | | X | |
| Product bottle | | | | | |
| Re-dispense Investigational | | | | | X |
| Product bottle | | | | | |
| Treatment administration | | | | X | X |
| instruction | | | | | |
| Collect unused | | | | | X |
| investigational product | | | | | |
| and used bottles; | | | | | |
| assess compliance | | | | | |
| Adverse event monitoring | | X | X | X | X |
| Concomitant medications | | X | X | X | X |

Washout Period Visit (if applicable; Weeks −14 to −6). The purpose of this visit was to discuss the study with a potential subject and obtain informed consent that is signed and dated before any procedures, including washout are performed. Subjects who agreed to discontinue current treatment began washout after the consent form was signed. A symptoms/complaints and medication diary was dispensed at this visit and the subject was instructed in how to complete the diary. Once the washout period was completed, the subject will return to the site for visit 1A.

The activities and assessments conducted during the visit included: informed consent; demographics; medical/gynecological history; collection of prior and concomitant medication information; height, body weight measurement and BMI calculation; collection of vital signs (body temperature, HR, RR, and BP); dispensation of symptoms/complaints diary and instruction in how to complete the diary

Screening Period Visits (Visits 1A and 1B). Subjects not requiring washout begin screening procedures at visit 1A as described above for the washout period. With the exception of vital signs, procedures performed at washout will not be repeated at screening visit 1A. In general, screening visits 1A and 1B were completed within 6 weeks (42 days) for subjects without a uterus or within 8 weeks (56 days) for subjects with a uterus. All screening assessments were completed prior to randomization. The investigators reviewed the results from all screening procedures and determined if the subjects were eligible for enrollment into the study.

Visit 1A (approximately Week −6 to 0). Visit 1A was conducted after the wash-out period (if applicable) or after the subject provided informed consent. The subject was advised to fast for 8 hours prior to the visit for blood draws.

Procedures and evaluations conducted at the visit included: informed consent; demographics; medical/gynecological history; collection of the symptoms/complaints and medications diary from washout (if applicable) and review with the subject; recording of prior medication information; recording and assessment of adverse events (AEs) starting from the signing of informed consent; height, body weight measurement and BMI calculation; collection of vital signs (body temperature, HR, RR, and BP); physical examination; breast examination (including a mammogram conducted up to nine months prior to Visit 2); urine pregnancy test as required; blood and urine sample collection for blood chemistry (minimum fast of 8 hrs), hematology, and urinalysis; serum FSH as required; 12-Lead ECG.

At visit 1A, the VVA symptom self-assessment questionnaire was conducted and most bothersome symptoms were identified, with the subject self-identifying moderate or severe pain with sexual activity as her MBS to continue screening. The symptoms/complaints and medications diary was dispensed, and subjects were instructed in how to complete the diary. Subjects were instructed to refrain from use of vaginal lubricants for 7 days and sexual intercourse/vaginal douching for 24 hours prior to the vaginal pH assessment to be done at visit 1B.

Visit 1B (approximately Week −4 to Week 0). Visit 1B was conducted after the subject's initial screening visit and after the other screening results indicated that the subject was otherwise an eligible candidate for the study (preferably around the middle of the screening period).

Procedures and evaluations conducted at the visit included: VVA symptom self-assessment questionnaire, the subject having indicated moderate to severe pain with sexual activity with vaginal penetration in order to continue screening; collection of vital signs (body temperature, HR, RR, and BP); pelvic examination; investigator assessment of vaginal mucosa as described above; assessment of vaginal pH (sexual intercourse or vaginal douching within 24 hrs prior to the assessment being prohibited, and a subject's vaginal pH being >5.0 to continue screening); Pap smear; vaginal cytological smear (one repetition being permitted during screening if no results were obtained from the first smear); endometrial biopsy performed as described above; review of the symptoms/complaints and medications diary with the subject.

Visit 2 (Week 0; Randomization/Baseline). Subjects who met entry criteria were randomized to investigational product at this visit. Procedures and evaluations conducted at the visit included: self-administration of FSFI by subjects not participating in the PK substudy; review of the symptoms/complaints and medications diary with the subject; review of evaluations performed at screening visits and verification of present of all inclusion criteria and the absence of all exclusion criteria; collection of vital signs (body temperature, HR, RR, and BP); randomization, with subjects meeting all entry criteria being randomized and allocated a bottle number; dispensation of investigational product and instruction in how to insert the capsule vaginally, with subjects receiving their first dose of investigational product under supervision; dispensation of dosing diary and instruction on completion of the treatment diary, including recording investigational product usage and sexual activity.

Visit 3 (Week 2, Day 14±3 days). Procedures and evaluations conducted at the visit included: completion of the VVA symptom self-assessment questionnaire; review of the symptoms/complaints and medications diaries with the subject; collection of vital signs (body temperature, HR, RR, and BP); Assessment of vaginal mucosa; assessment of vaginal pH (with sexual intercourse or vaginal douching within 24 hrs prior to the assessment being prohibited); vaginal cytological smear; collection of unused investigational product and bottle for assessment of compliance/accountability; re-dispensation of investigational product and re-instruction in how to insert the capsule vaginally if necessary; review of the completed dosing diary with the subject.

Visit 4 (Week 6, Day 42±3 days). Procedures and evaluations conducted at the visit included: completion of the VVA symptom self-assessment questionnaire; review of the symptoms/complaints and medications diary with the subject; collection of vital signs (body temperature, HR, RR, and BP); assessment of vaginal mucosa as described above; vaginal cytological smear; assessment of vaginal pH (with sexual intercourse or vaginal douching within 24 hrs prior to the assessment being prohibited); collection of unused investigational product for assessment of compliance/accountability; re-dispensation of investigational product and re-instruction in how to insert the capsule vaginally if necessary; review of the completed dosing diary with the subject.

Visit 5 (Week 8, Day 56±3 days). Procedures and evaluations conducted at the visit included: completion of the VVA symptom self-assessment questionnaire; review of the symptoms/complaints and medications diary with the subject; collection of vital signs (body temperature, HR, RR, and BP); assessment of vaginal mucosa as described above; vaginal cytological smear; assessment of vaginal pH (with sexual intercourse or vaginal douching within 24 hrs prior to the assessment being prohibited); collection of unused investigational product for assessment of compliance/accountability; re-dispensation of investigational product and re-instruction in how to insert the capsule vaginally if necessary; review of the completed dosing diary with the subject.

Visit 6 (Week 12, Day 84±3 days or early termination). This visit was performed if a subject withdraws from the study before visit 6. Procedures performed at this visit included: completion of the VVA symptom self-assessment questionnaire; review of the subject the dosing diary, symptoms/complaints, and medications diaries with the subject; collection of blood and urine sample collection for blood chemistry (minimum fast of 8 hrs), hematology, and urinalysis; collection of vital signs (body temperature, HR, RR, and BP) and weight; performance of 12-lead-ECG; collection of unused investigational product and container for assessment of compliance/accountability; physical examination; breast exam; assessment of vaginal mucosa as described above; assessment of vaginal pH (with sexual intercourse or vaginal douching within 24 hrs prior to the assessment being prohibited); vaginal cytological smear; Pap smear; endometrial biopsy; self-administration of FSFI by subjects not participating in the PK substudy; self-administration of survey titled “Acceptability of product administration Survey” by subjects.

Follow-up Interview (approximately 15 days after the last dose of investigational product). Each subject who received investigational product received a follow-up phone call, regardless of the duration of therapy, approximately 15 days following the last dose of investigational product. The follow-up generally took place after receipt of all safety assessments (e.g., endometrial biopsy and mammography results). The follow-up included: review of ongoing adverse events and any new adverse events that occurred during the 15 days following the last dose of investigational product; review of ongoing concomitant medications and any new concomitant medications that occurred during the 15 days following the last dose of investigational product; and discussion of all end of study safety assessments and determination if further follow up or clinic visit is required.

### PK Substudy Visit Procedures and Schedule

Screening Visit 1A. In addition to the procedures listed described above, activities in the PK substudy also included: provision of informed consent by subject and agreement to participate in the PK substudy; collection of a serum blood sample during the visit for baseline assessment of estradiol, estrone, and estrone conjugates.

Visit 2 (Week 0, Day 1). In addition to the procedures listed described above, activities in the PK substudy also included collection of serum blood sample obtained prior to the administration of investigational product (timepoint 0 h) for baseline assessment of estradiol, estrone, estrone conjugates, and SHBG. The investigational product was self-administered by the subject after the pre-treatment blood sample has been taken. After investigational product administration, serum blood samples were obtained at 2 h, 4 h, 6 h, 10 h, and 24 h timepoints for estradiol, estrone, and estrone conjugates (serum samples were generally taken within +/−5 minutes at 2 h and 4 h, within +/−15 minutes at 6 h, and within +/−1 h at 10 h and 24 h). The subject was released from the site after the 10 hour sample and instructed to return to the site the next morning for the 24 hour blood draw. The subject was instructed not to self-administer the day 2 dose until instructed by the site personnel to dose at the clinical site. The subject was released from the clinical site following the 24 hour blood sample and administration of the day 2 dose.

Visit 3 (Week 2, Day 14). The visit must occurred on day 14 with no visit window allowed. In addition to the procedures listed above, the PK substudy included collection of a serum blood sample prior to the administration of day 14 dose (timepoint 0 h) for SHBG and PK assessments. The subject self-administered the day 14 dose at the clinical site, and serum blood samples were obtained at 2 h, 4 h, 6 h, 10 h, and 24 h timepoints for estradiol, estrone, and estrone conjugates. The subject was released from the site after the 10 hour sample and instructed to return to the site the next morning for the 24 hour blood draw. The subject was instructed not to self-administer the day 15 dose until instructed by the site personnel to dose at the clinical site. The subject was released from the clinical site following the 24 hour blood sample and administration of the day 15 dose. The subject was be instructed to administer the next dose of study drug on day 18 or day 19 and continue dosing on a bi-weekly basis at the same time of day for each dose.

Visit 6 (Week 12, Day 84±3 days, or at early termination). The visit took place 4 days after last IP dose or early termination. A serum sample for estradiol, estrone, and estrone conjugates and SHBG was drawn in addition to the procedures described above.

PK sub-study visits were typically conducted so as to include the activities outlined in Table 44.

TABLE 44
Schedule of Assessments for PK Sub-study

| | | | | Visit 2: | |
|---|---|---|---|---|---|
| | | Visit 1A | Visit 1B | Randomization/ | Visit 3: |
| | Washout | Screening | Screening | Baseline | Interim |
| | | | | | Week 2 |
| | Week −14 | | Week −4 to | Week 0 | Day 14 |
| Activity | to −6 | Week −6 to 0 | 0 | Day 1 | (no window) |
| PK sub-study Informed | X | X | | | |
| consent | | | | | |
| Demographics/Medical | X | X | | | |
| and Gynecological history | | | | | |
| and prior medications | | | | | |
| Weight | X | X | | | |
| Height and BMI | X | X | | | |
| calculation | | | | | |
| Vital signs | X | X | X | X | X |
| MBS | | X | | | |
| Subject VVA Self- | | X | X | | X |
| Assessment | | | | | |
| Questionnaire | | | | | |
| Physical examination | | X | | | |
| including breast exam | | | | | |
| Laboratory safety tests | | X | | | |
| (Hematology, Serum | | | | | |
| Chemistry, FSHP, | | | | | |
| Urinalysis) | | | | | |
| PK Serum Blood Samples | | X | | X | X |
| (Estradiol, Estrone, | | | | | |
| Estrone Conjugates) | | | | | |
| Serum blood samples for | | | | X | X |
| SHBG | | | | | |
| 12-Lead ECG | | X | | | |
| Pelvic exam | | | X | | |
| Vaginal pH | | | X | | X |
| Papanicolaou (Pap) smear | | | X | | |
| Investigator assessment of | | | X | | X |
| vaginal mucosa | | | | | |
| Vaginal cytological smear | | | X | | X |
| Mammogram | | X | | | |
| Endometrial biopsy | | | X | | |
| Diary Dispense | X | X | X | X | X |
| Diary Collection | | X | X | X | X |
| Satisfaction Survey | | | | | |
| Urine pregnancy test | | X | | | |
| Randomization | | | | X | |
| Dispense new | | | | X | |
| Investigational Product | | | | | |
| (IP) bottle | | | | | |
| Re-dispense | | | | | X |
| Investigational Product | | | | | |
| (IP) bottle | | | | | |
| IP administration | | | | X | X |
| instruction | | | | | |
| Collect unused IP and | | | | | X |
| used bottles; assess | | | | | |
| compliance | | | | | |
| Adverse event monitoring | | X | X | X | X |
| Concomitant medications | | X | X | X | X |

Table 44-continued

| | | | Visit 6: End | |
|---|---|---|---|---|
| | Visit 4: | Visit 5: | of treatment | Telephone |
| | Interim | Interim | or Early Term | Interview |
| | | | | Week 14 |
| | Week 6 | Week 8 | Week 12 | approximately |
| | Day 42 | Day 56 | Day 84 (± 3 d) | 15 days after |
| Activity | (± 3 d) | (± 3 d) | (4 days after last IP dose) | last dose of IP |
| PK sub-study Informed | | | | |
| consent | | | | |
| Demographics/Medical | | | | |
| and Gynecological | | | | |
| history and prior | | | | |
| medications | | | | |
| Weight | | | X | |
| Height and BMI | | | | |
| calculation | | | | |
| Vital signs | X | X | X | |
| MBS | | | | |
| Subject VVA Self- | X | X | X | |
| Assessment | | | | |
| Questionnaire | | | | |
| Physical examination | | | X | |
| including breast exam | | | | |
| Laboratory safety tests | | | X | |
| (Hematology, Serum | | | | |
| Chemistry, FSHP, | | | | |
| Urinalysis | | | | |
| PK Serum Blood | | | X | |
| Samples (Estradiol, | | | | |
| Estrone, Estrone | | | | |
| Conjugates) | | | | |
| Serum blood samples | | | X | |
| for SHBG | | | | |
| 12-Lead ECG | | | X | |
| Pelvic exam | | | X | |
| Vaginal pH | X | X | X | |
| Papanicolaou (Pap) | | | X | |
| smear | | | | |
| Investigator assessment | X | X | X | |
| of vaginal mucosa | | | | |
| Vaginal cytological | X | X | X | |
| smear | | | | |
| Mammogram | | | | |
| Endometrial biopsy | | | X | |
| Diary Dispense | X | X | | |
| Diary Collection | X | X | X | |
| Satisfaction Survey | | | X | |
| Urine pregnancy test | | | | |
| Randomization | | | | |
| Dispense new | | X | | |
| Investigational Product | | | | |
| (IP) bottle | | | | |
| Re-dispense | X | | | |
| Investigational Product | | | | |
| (IP) bottle | | | | |
| IP adminstration | X | X | | |
| instruction | | | | |
| Collect unused IP and | X | X | X | |
| used bottles; assess | | | | |
| compliance | | | | |
| Adverse event | X | X | X | X |
| monitoring | | | | |
| Concomitant | X | X | X | X |
| medications | | | | |

An Adverse Event (AE) in the study was defined as the development of an undesirable medical condition or the deterioration of a pre-existing medical condition following or during exposure to a pharmaceutical product, whether or not considered casually related to the product. An AE could occur from overdose of investigational product. In this study, an AE can include an undesirable medical condition occurring at any time, including baseline or washout periods, even if no study treatment has been administered. Relationship to Investigational Product

The investigators determined the relationship to the investigational product for each AE (Not Related, Possibly Related, or Probably Related). The degree of “relatedness” of the adverse event to the investigational product was described as follows: not related—no temporal association and other etiologies are likely the cause; possible—temporal association, but other etiologies are likely the cause. However, involvement of the investigational product cannot be excluded; probable—temporal association, other etiologies are possible but unlikely. The event may respond if the investigational product is discontinued.

### Example 11: Efficacy Results of Randomized, Double-Blind, Placebo-Controlled Multicenter Study

Each of the three doses showed statistical significance compared with placebo for the primary endpoints. Each of the three doses showed statistical significance compared with placebo for the secondary endpoints. Table 45 shows the statistical significance of the experimental data for each of the four co-primary endpoints. Each of the dosages met each of the four co-primary endpoints at a statistically significant level. The 25 mcg dose of TX-004HR demonstrated highly statistically significant results at the p≤0.0001 level compared to placebo across all four co-primary endpoints. The 10 mcg dose of TX-004HR demonstrated highly statistically significant results at the p≤0.0001 level compared to placebo across all four co-primary endpoints. The 4 mcg dose of TX-004HR also demonstrated highly statistically significant results at the p≤0.0001 level compared to placebo for the endpoints of superficial vaginal cells, parabasal vaginal cells, and vaginal pH; the change from baseline compared to placebo in the severity of dyspareunia was at the p=0.0255 level.

TABLE 45
Statistical Significance of Results for Co-Primary Endpoints (Based on
Mean Change from Baseline to Week 12 Compared to Placebo)

| | 25 mcg | 10 mcg | 4 mcg |
|---|---|---|---|
| Superficial Cells | P < 0.0001 | P < 0.0001 | P < 0.0001 |
| Parabasal Cells | P < 0.0001 | P < 0.0001 | P < 0.0001 |
| Vaginal pH | P < 0.0001 | P < 0.0001 | P < 0.0001 |
| Severity of | P = 0.0001 | P = 0.0001 | P = 0.0255 |
| Dyspareunia | | | |

Statistical improvement over placebo was also observed for all three doses at the first assessment at week two and sustained through week 12. The pharmacokinetic data for all three doses demonstrated low systemic absorption, supporting the previous Phase 1 trial data. TX-004HR was well tolerated, and there were no clinically significant differences compared to placebo-treated women with respect to adverse events. There were no drug-related serious adverse events reported.

As shown in the data below, in the MITT population (n=747) at week 12, all TX-004HR doses compared with placebo significantly decreased the percentage of parabasal cells and vaginal pH, significantly increased the percentage of superficial cells, and significantly reduced the severity of dyspareunia (all p≤0.00001 except dyspareunia at 4 μg p=0.0149).

At weeks 2, 6, and 8, the percentage of parabasal cells and vaginal pH significantly decreased p<0.00001); the percentage of superficial cells significantly increased (p<0.00001); and the severity of dyspareunia significantly improved from baseline with all TX-004HR doses vs placebo (4 μg p<0.03; 10 μg and 25 μg p<0.02).

Moderate-to-severe vaginal dryness was reported by 93% at baseline and significantly improved (p<0.02) for all doses at weeks 2, 6, 8, and 12 (except 4 μg at week 2). Vulvar and/or vaginal itching or irritation significantly improved (p<0.05) for 10 μg at weeks 8 and 12, and for 25 μg at week 12.

TX-004HR was well tolerated, had high acceptability, and no treatment-related serious AEs were reported in the safety population (n=764). There were no clinically significant differences in any AEs or treatment-related SAEs between TX-004HR and placebo. Very low to negligible systemic levels of estradiol were observed.

All TX-004HR doses were safe and effective and resulted in very low to negligible systemic absorption of E2 in women with VVA and moderate-to-severe dyspareunia. Onset of effect was seen as early as 2 weeks and was maintained throughout the study and acceptability was very high. This novel product provides a promising new treatment option for women experiencing menopausal VVA.

### Cytology

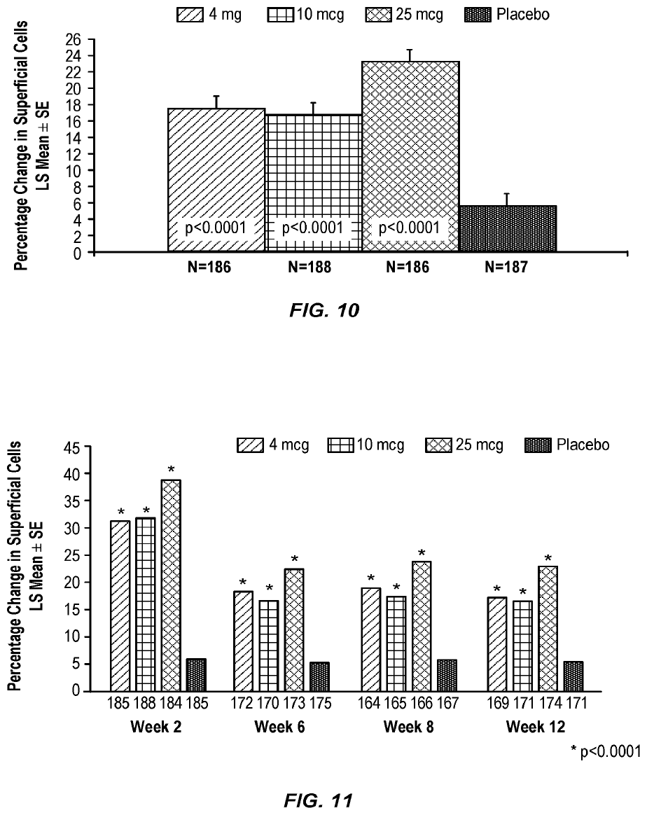

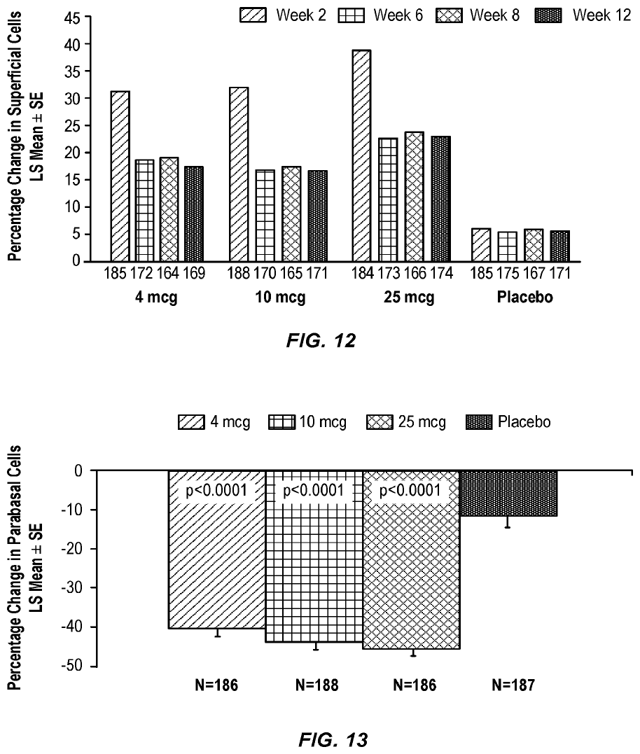

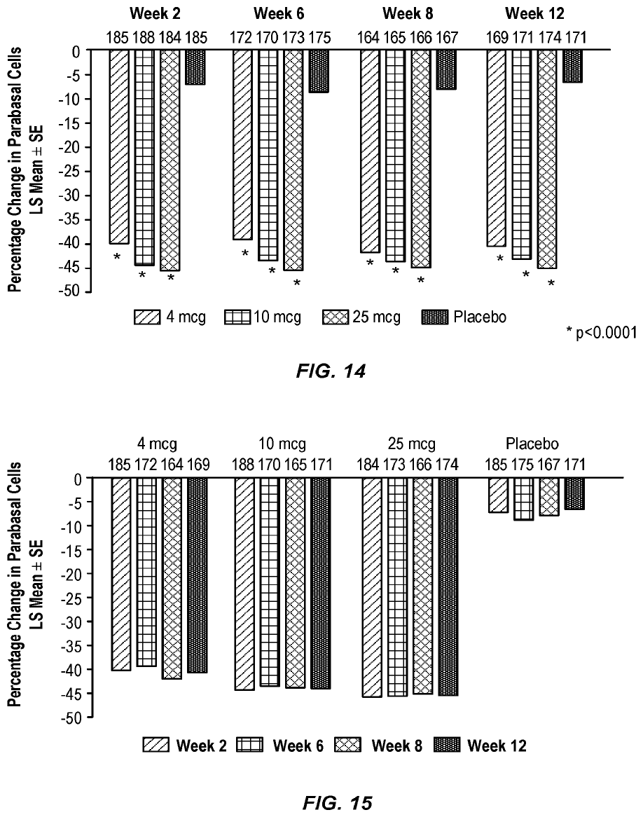

Vaginal cytology data was collected as vaginal smears from the lateral vaginal walls according to procedures presented above to evaluate vaginal cytology at screening and Visit 6—End of treatment (day 84). The change in the Maturation Index was assessed as a change in cell composition measured at Visit 1—Baseline (day 1) compared to the cell composition measured at Visit 3—End of treatment (day 84). The change in percentage of superficial, parabasal, and intermediate cells obtained from the vaginal mucosal epithelium from a vaginal smear was recorded. Results from these assessments for superficial cells are presented in Table 46 and Table 47, as well as FIG. 10, FIG. 11, and FIG. 12. Results from these assessments for parabasal cells are presented in Table 48 and Table 49, as well as FIG. 13, FIG. 14, and FIG. 15.

### Superficial Cells

TABLE 46
Superficial Cells P-values by Treatment Week

| | 4 μg | 10 μg | 25 μg |
|---|---|---|---|
| Week 2 | < 0.0001 | < 0.0001 | < 0.0001 |
| Week 6 | < 0.0001 | < 0.0001 | < 0.0001 |
| Week 8 | < 0.0001 | < 0.0001 | < 0.0001 |
| Week 12 | < 0.0001 | < 0.0001 | < 0.0001 |

TABLE 47
Superficial Cells Change in Severity from Baseline by Treatment Week
(change in percent of total vaginal cells)

| | 4 μg | | 10 μg | | 25 μg | | Placebo | |
|---|---|---|---|---|---|---|---|---|
| Week 2 | 31.35 | (1.496) | 31.93 | (1.488) | 38.85 | (1.5) | 6.05 | (1.498) |
| Week 6 | 18.41 | (1.536) | 16.88 | (1.543) | 22.65 | (1.532) | 5.43 | (1.525) |
| Week 8 | 19.04 | (1.561) | 17.41 | (1.558) | 23.88 | (1.554) | 5.98 | (1.551) |
| Week 12 | 17.5 | (1.542) | 16.72 | (1.54) | 23.2 | (1.529) | 5.63 | (1.537) |

The study showed the formulations disclosed herein across all doses increased the percentage of superficial cells across all dosages in a statistically significant way.

### Parabasal Cells

TABLE 48
Parabasal Cells P-values by Treatment Week

| | 4 μg | 10 μg | 25 μg |
|---|---|---|---|
| Week 2 | < 0.0001 | < 0.0001 | < 0.0001 |
| Week 6 | < 0.0001 | < 0.0001 | < 0.0001 |
| Week 8 | < 0.0001 | < 0.0001 | < 0.0001 |
| Week 12 | < 0.0001 | < 0.0001 | < 0.0001 |

TABLE 49
Parabasal Cells Change in Severity from Baseline
by Treatment Week (change in percent of total vaginal cells)

| | 4 μg | | 10 μg | | 25 μg | | Placebo | |
|---|---|---|---|---|---|---|---|---|
| Week 2 | −40.23 | (1.719) | −44.42 | (1.708) | −45.6 | (1.723) | −7 | (1.72) |
| Week 6 | −39.36 | (1.75) | −43.55 | (1.752) | −45.61 | (1.746) | −9.23 | (1.741) |
| Week 8 | −41.87 | (1.768) | −43.78 | (1.764) | −45.08 | (1.762) | −7.86 | (1.76) |
| Week 12 | −40.63 | (1.755) | −44.07 | (1.751) | −45.55 | (1.745) | −6.73 | (1.75) |

The increase of superficial cells and decrease of parabasal cells showed statistical significance over placebo at week 2 and for every week thereafter, including at week 12. Administration of the pharmaceutical formulation resulted in rapid onset of action, as early as two weeks after the initial administration. Rapid onset of action may be coupled with the rapid absorption demonstrated in the pharmacokinetic data presented below.

pH

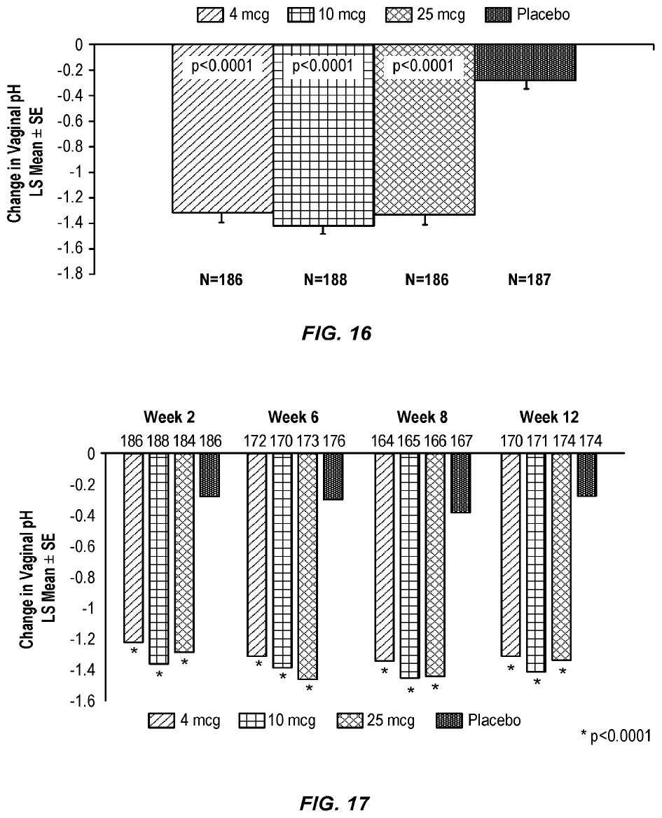

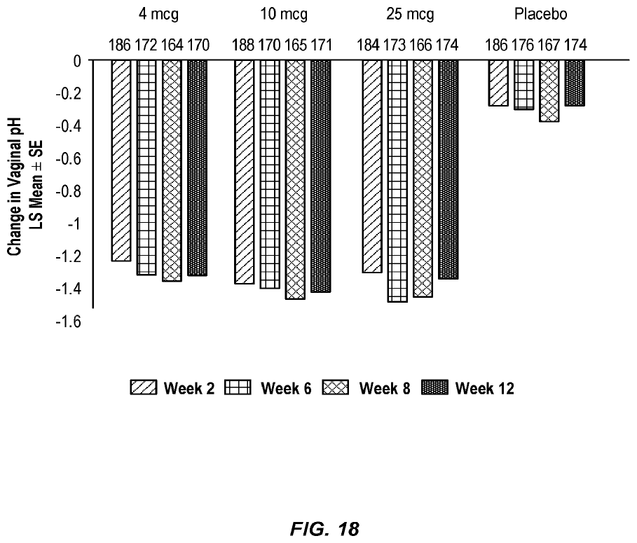

Vaginal pH was measured at Screening and Visit 6—End of treatment (day 84). The pH measurement was obtained as disclosed herein. Results from these assessments are presented in Table 50 and Table 51, and FIG. 16, FIG. 17, and FIG. 18.

TABLE 50
pH P-values by Treatment Week

| | 4 μg | 10 μg | 25 μg |
|---|---|---|---|
| Week 2 | < 0.0001 | < 0.0001 | < 0.0001 |
| Week 6 | < 0.0001 | < 0.0001 | < 0.0001 |
| Week 8 | < 0.0001 | < 0.0001 | < 0.0001 |
| Week 12 | < 0.0001 | < 0.0001 | < 0.0001 |

TABLE 51
pH Changein Severity from Baseline by
Treatment Week (change in pH)

| | 4 μg | | 10 μg | | 25 μg | | Placebo | |
|---|---|---|---|---|---|---|---|---|
| Week 2 | −1.23 | (0.064) | −1.37 | (0.064) | −1.3 | (0.065) | −0.28 | (0.064) |
| Week 6 | −1.32 | (0.066) | −1.4 | (0.066) | −1.48 | (0.066) | −0.3 | (0.065) |
| Week 8 | −1.35 | (0.067) | −1.46 | (0.067) | −1.45 | (0.066) | −0.38 | (0.066) |
| Week 12 | −1.32 | (0.066) | −1.42 | (0.066) | −1.34 | (0.066) | −0.28 | (0.066) |

The decrease in vaginal pH was observed at statistically significant levels at week 2 and through the end of the study. Surprisingly, the pH decreased in all three pharmaceutical formulations tested and at all three dosages of over a full pH unit for all three doses.

### Most Bothersome Symptoms

### Dyspareunia

Subjects were asked to specify the symptom that she identified as the “most bothersome symptom.” During the screening period all of the subjects were provided with a questionnaire to self-assess the symptoms of VVA: (1) dyspareunia; (2) vaginal dryness; and (3) vaginal or vulvar irritation, burning, or itching. Each symptom was measured on a scale of 0 to 3, where 0=none, 1=mild, 2=moderate, and 3=severe. Each subject was given a questionnaire at each visit and the responses were recorded. All randomized subjects were also provided a questionnaire to self-assess the symptoms of VVA at Visit 1 and on each subsequent visit through Visit 6—End of the treatment (day 84). Subjects recorded their self-assessments daily in a diary and answers were collected on visits 8 and 15 (end of treatment). Pre-dose evaluation results obtained at Visit 1 were considered as baseline data for the statistical analyses. Data from these assessments for dyspareunia are presented in Table 52 and Table 53. Data from these assessments for dryness are presented in Table 54 and Table 55.

TABLE 52
Dyspareunia P-values by Treatment Week

| | 4 μg | 10 μg | 25 μg |
|---|---|---|---|
| Week 2 | 0.026 | 0.0019 | 0.0105 |
| Week 6 | 0.0069 | 0.0009 | <0.0001 |
| Week 8 | 0.0003 | <0.0001 | <0.0001 |
| Week 12 | 0.0149 | <0.0001 | <0.0001 |

TABLE 53
Dyspareunia Change in Severity from Baseline
by Treatment Week (0 to 3 severity scale)

| | 4 μg | | 10 μg | | 25 μg | | Placebo | |
|---|---|---|---|---|---|---|---|---|
| Week 2 | −0.99 | (0.072) | −1.08 | (0.072) | −1.02 | (0.073) | −0.76 | (0.072) |
| Week 6 | −1.3 | (0.072) | −1.37 | (0.072) | −1.48 | (0.072) | −1.03 | (0.07) |
| Week 8 | −1.52 | (0.073) | −1.64 | (0.074) | −1.62 | (0.075) | −1.15 | (0.072) |
| Week 12 | −1.52 | (0.071) | −1.69 | (0.071) | −1.69 | (0.071) | −1.28 | (0.07) |

Each of the 4 μg, 10 μg, and 25 μg formulations tests demonstrated an early onset of action at week 2 for the most bothersome symptom of dyspareunia, evidenced by the statistically significant results (measured by p-value) in Table 52. After two weeks, each dose demonstrated separation from placebo in improvement in the most bothersome symptom of dyspareunia.

Coupled with the PK data presented below, these results show that the formulations disclosed herein provide a bolus of estradiol within two hours of administration, which resulted in a decrease in the severity of dyspareunia as early as two weeks later. Estradiol is rapidly absorbed at around two hours, which is significantly faster than the formulations of the prior art that sought an extended release profile. The rapid absorption of estradiol is believed to be a result of administration with a liquid formulation.

Surprisingly, the 4 μg formulation showed clinical effectiveness at two weeks along with the 25 μg and 10 μg dosage levels. These data demonstrate that 4 μg is an effective dose, and can be effective as early as two weeks after administration for the most bothersome symptom of dyspareunia.

### Dryness

TABLE 54
Dryness P-values by Treatment Week

| | 4 μg | 10 μg | 25 μg |
|---|---|---|---|
| Week 2 | 0.1269 | 0.0019 | 0.0082 |
| Week 6 | 0.0094 | 0.0001 | 0.0005 |
| Week 8 | 0.0128 | <0.0001 | 0.0008 |
| Week 12 | 0.0014 | <0.0001 | <0.0001 |

TABLE 55
Dryness Change in Severity from Baseline by Treatment Week
(0 to 3 severity scale)

| | 4 μg | 10 μg | 25 μg | Placebo |
|---|---|---|---|---|
| Week 2 | −0.86(0.066) | −1.01(0.065) | −0.96(0.066) | −0.72(0.066) |
| Week 6 | −1.14(0.067) | −1.27(0.068) | −1.23(0.067) | −0.9(0.067) |
| Week 8 | −1.25(0.069) | −1.44(0.068) | −1.34(0.068) | −1.01(0.068) |
| Week 12 | −1.27(0.068) | −1.47(0.067) | −1.47(0.067) | −0.97(0.067) |

Each of the 4 μg, 10 μg, and 25 μg formulations tests demonstrated an early onset of action at week 2 for the most bothersome symptom of dryness, evidenced by the statistically significant results (measured by p-value) in Table 54. After two weeks, each dose demonstrated separation from placebo in improvement in the most bothersome symptom of dryness.

### Irritation/Itching

TABLE 56
Irritation/Itching P-values by Treatment Week

| | 4 μg | 10 μg | 25 μg |
|---|---|---|---|
| Week 2 | 0.9616 | 0.2439 | 0.6518 |
| Week 6 | 0.7829 | 0.2328 | 0.4118 |
| Week 8 | 0.0639 | 0.0356 | 0.0914 |
| Week 12 | 0.0503 | 0.0055 | 0.0263 |

TABLE 57
Irritation/Itching Change in Severity from Baseline by Treatment Week
(0 to 3 severity scale)

| | 4 μg | 10 μg | 25 μg | Placebo |
|---|---|---|---|---|
| Week 2 | −0.47(0.054) | −0.56(0.053) | −0.51(0.054) | −0.47(0.054) |
| Week 6 | −0.57(0.055) | −0.64(0.055) | −0.61(0.055) | −0.55(0.055) |
| Week 8 | −0.74(0.056) | −0.76(0.056) | −0.73(0.056) | −0.59(0.056) |
| Week 12 | −0.75(0.055) | −0.81(0.055) | −0.77(0.055) | −0.6(0.055) |

Vulvar and/or vaginal itching or irritation significantly improved (p<0.05) for 10 μg at weeks 8 and 12, and for 25 μg at week 12. Moreover, the trend for 4 μg was an improvement in itching week over week to nearly being statistically significant at week 12.

Coupled with the PK data presented below, these results show that the formulations disclosed herein provide a bolus of estradiol within two hours of administration, which resulted in a decrease in the severity of dryness as early as two weeks later. Estradiol is rapidly absorbed at around two hours, which is significantly faster than the formulations of the prior art that sought an extended release profile. The rapid absorption of estradiol is believed to be a result of administration with a liquid formulation.

Surprisingly, the 4 μg formulation showed clinical effectiveness at two weeks along with the 25 μg and 10 μg dosage levels. These data demonstrate that 4 μg is an effective dose, and can be effective as early as two weeks after administration for the most bothersome symptom of dryness.

As described above, each dose was compared with placebo for change from baseline to week 12 in the percentages of vaginal superficial cells and parabasal cells, vaginal pH, and severity of dyspareunia (co-primary endpoints). The proportion of responders (defined as women with ≥2 of the following at week 12: vaginal superficial cells >5%, vaginal pH<5.0, ≥1 category improvement from baseline dyspareunia score) was compared in TX-004HR groups vs placebo. Pre-specified subgroup analyses of co-primary endpoints were analyzed by age (≤56 years, 57-61 years, and ≥62 years), BMI (≤24 kg/m², 25-28 kg/m², and ≥29 kg/m²), uterine status, parity, and vaginal births. Pharmacokinetic (PK) parameters were compared with placebo in a sub-analysis of the main study.

The proportion of responders was significantly higher for all TX-004HR dose groups vs placebo (p<0.0001 for all). All TX-004HR doses vs placebo significantly improved percentage of superficial and parabasal cells, vaginal pH, and severity of dyspareunia at 12 weeks. Subgroup analyses showed generally similar results for percentage of superficial and parabasal cells and vaginal pH irrespective of age, BMI, uterine status, parity, and vaginal births. Severity of dyspareunia was significantly reduced at 12 weeks with all TX-004HR doses vs placebo in most subgroups (Table 57A).

The PK sub-analysis (n=72), described in more detail below, found AUC and C_{avg} parameters for E2 and estrone (E1) with 4 μg and 10 μg TX-004HR to be similar to placebo. Increases occurred in E2 AUC and C_{avg} with 25 μg vs placebo but remained within the normal postmenopausal range. E2 levels at day 84 were similar between the TX-004HR groups and placebo, indicating no systemic drug accumulation.

All doses of TX-004HR were associated with robust efficacy and demonstrated a statistically significant difference vs placebo for increasing superficial cells, decreasing parabasal cells and vaginal pH, and reducing the severity of dyspareunia. Age, BMI, uterine status, parity and vaginal births generally did not affect TX-004HR efficacy. These results occurred with negligible systemic absorption of TX-004HR estradiol doses of 4 μg, 10 μg, and 25 μg.

TABLE 57A
Change from baseline to week 12 in the severity of dyspareunia
(LS mean change ± SE).

| | | | | TX-004HR | | TX-004HR | | TX-004HR | |
|---|---|---|---|---|---|---|---|---|---|
| | | Placebo | | 4 μg | | 10 μg | | 25 μg | |
| Key clinical factors | | (n = 187) | | (n = 186) | | (n = 188) | | (n = 186) | |
| Age, years | ≤56 | n = 52 | −1.25 ± 0.119 | n = 50 | −1.58 ± 0.122 | n = 61 | −1.77 ± 0.112^{†} | n = 65 | −1.86 ± 0.108^{‡} |
| | 57-61 | n = 53 | −1.39 ± 0.118 | n = 50 | −1.42 ± 0.121 | n = 49 | −1.63 ± 0.121 | n = 47 | −1.79 ± 0.125* |
| | ≥62 | n = 58 | −1.19 ± 0.122 | n = 51 | −1.52 ± 0.126 | n = 44 | −1.66 ± 0.138^{†} | n = 47 | −1.38 ± 0.135 |
| BMI, | ≤24 | n = 56 | −1.14 ± 0.115 | n = 58 | −1.48 ± 0.113* | n = 56 | −1.6 ± 0.117^{†} | n = 51 | −1.72 ± 0.123^{‡} |
| kg/m² | 25 to 28 | n = 57 | −1.48 ± 0.118 | n = 45 | −1.51 ± 0.131 | n = 52 | −1.78 ± 0.124 | n = 58 | −1.77 ± 0.117 |
| | ≥29 | n = 50 | −1.21 ± 0.125 | n = 48 | −1.56 ± 0.125 | n = 46 | −1.71 ± 0.129^{†} | n = 50 | −1.57 ± 0.124* |
| Uterine | Intact | n = 101 | −1.35 ± 0.086 | n = 82 | −1.66 ± 0.095* | n = 84 | −1.74 ± 0.095^{†} | n = 85 | −1.81 ± 0.094^{‡} |
| status | Non-intact | n = 62 | −1.15 ± 0.115 | n = 69 | −1.35 ± 0.108 | n = 70 | −1.63 ± 0.108^{†} | n = 74 | −1.55 ± 0.107* |
| Pregnancy | Pregnancy = 0 | n = 16 | −1.18 ± 0.220 | n = 17 | −1.28 ± 0.217 | n = 19 | −1.26 ± 0.209 | n = 13 | −1.64 ± 0.257 |
| status | Pregnancy ≥ 1 | n = 147 | −1.28 ± 0.073 | n = 134 | −1.55 ± 0.075* | n = 135 | −1.74 ± 0.076^{§} | n = 146 | −1.70 ± 0.073^{‡} |
| Vaginal | Vaginal birth = 0 | n = 26 | −1.19 ± 0.171 | n = 22 | −1.74 ± 0.189* | n = 29 | −1.68 ± 0.161* | n = 31 | −1.76 ± 0.160* |
| births | Vaginal birth ≥ 1 | n = 121 | −1.30 ± 0.080 | n = 112 | −1.51 ± 0.082 | n = 106 | −1.77 ± 0.085^{‡} | n = 115 | −1.69 ± 0.082^{‡} |

*p < 0.05;
^{†}p < 0.01;
^{‡}p < 0.001;
^{§}p < 0.0001 vs placebo.

Visual evaluation of the vaginal epithelium, a secondary endpoint of the trial, was performed during gynecological examinations at baseline and weeks 2, 6, 8, and 12. A four-point score (0=none, 1=mild, 2=moderate, 3=severe) was used to assess changes in vaginal color, vaginal epithelial integrity, vaginal epithelial surface thickness, and vaginal secretions. Change from baseline to each time point was compared with placebo using the mixed effect model repeat measurement (MMRM) analysis.

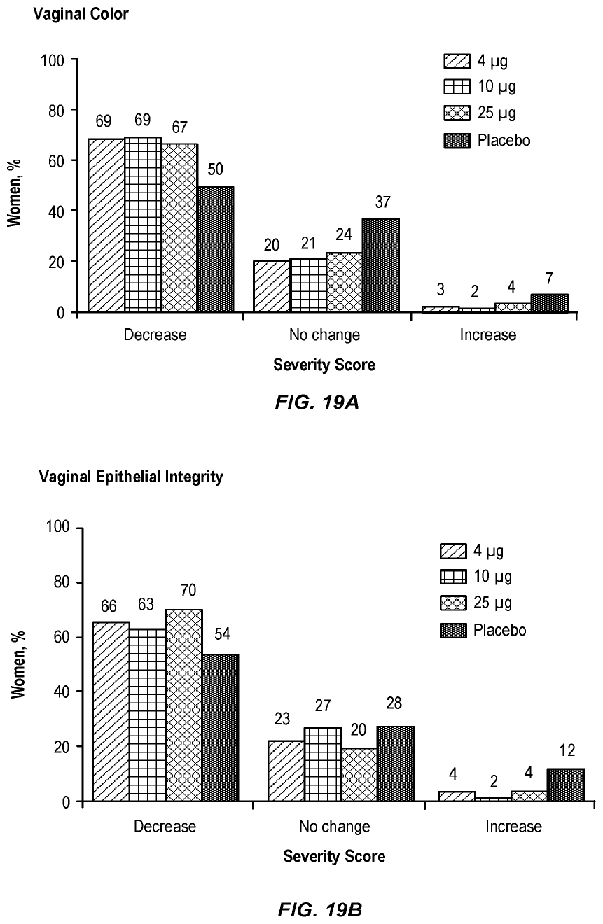

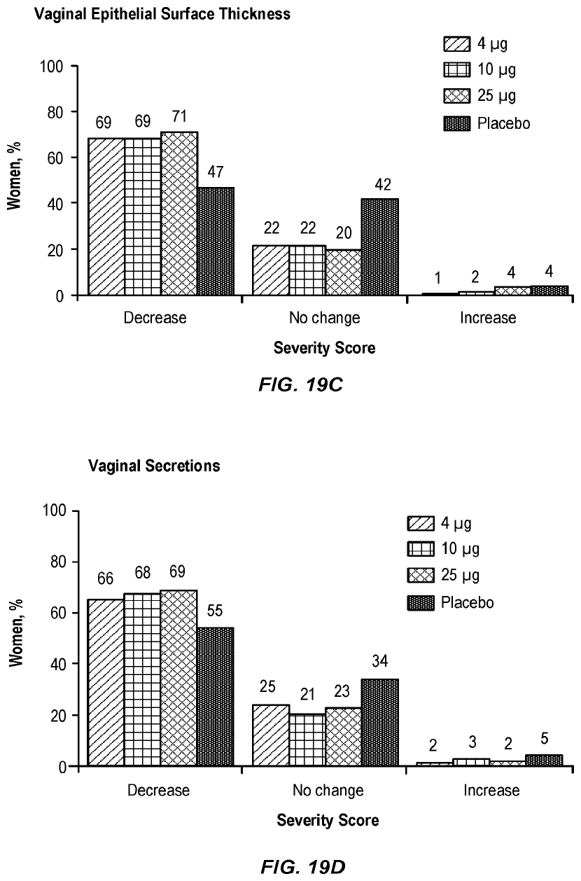

At baseline, women had mean scores of 1.8 for vaginal color, 1.5 for epithelial integrity, 1.9 for epithelial surface thickness, and 1.7 for secretions. These scores were consistent with VVA reflecting pallor, diminished vaginal wall integrity and thickness, and secretions. Significant improvements from baseline at weeks 2, 6, 8 and 12 (Table 57B; FIG. 19A-FIG. 19D) were observed for all 3 doses of TX-004HR compared with placebo in vaginal color (white to pink), epithelial integrity, epithelial surface thickness and secretions (p<0.001 for all). After 12 weeks, women in the active TX-004HR treatment groups had mean scores less than 1 in all four characterized categories. Vaginal visual examination of women in the 3 TX-004HR groups had greater reported improvements from baseline in all vaginal parameters examined than placebo subjects and at all time points. These improved vaginal visual scores reflect other observed measures of efficacy of TX-004HR (4 μg, 10 μg, and 25 μg) at treating moderate-to-severe VVA in postmenopausal women, with negligible to very low systemic E2 absorption,

TABLE 57B
Change from baseline at week 12 in vaginal parameters

| | | TX-004HR | TX-004HR | TX-004HR | |
|---|---|---|---|---|---|
| | | 4 μg | 10 μg | 25 μg | Placebo |
| Vaginal Parameters, mean (SD) | | (n = 171) | (n = 173) | (n = 175) | (n = 175) |
| Vaginal epithelial | Baseline | 1.8 (0.61) | 1.7 (0.59) | 1.8 (0.60) | 1.7 (0.64) |
| color | 12 weeks | 0.8 (0.67) | 0.7 (0.64) | 0.8 (0.68) | 1.2 (0.80) |
| | Change | −1.0 (0.82) | −1.1 (0.80) | −1.0 (0.88) | −0.6 (0.83) |
| | LS Mean (SE) | −0.97 (0.05)* | −1.06 (0.05)* | −0.96 (0.05)* | −0.60 (0.05) |
| Vaginal epithelial | Baseline | 1.6 (0.84) | 1.4 (0.83) | 1.5 (0.77) | 1.5 (0.84) |
| integrity | 12 weeks | 0.5 (0.69) | 0.4 (0.57) | 0.5 (0.66) | 0.9 (0.91) |
| | Change | −1.0 (0.93) | −1.0 (0.89) | −1.0 (0.91) | −0.6 (0.98) |
| | LS Mean (SE) | −0.97 (0.05)* | −1.07 (0.05)* | −1.01 (0.05)* | −0.60 (0.05) |
| Vaginal epithelial | Baseline | 1.9 (0.67) | 1.8 (0.63) | 1.9 (0.59) | 1.9 (0.65) |
| surface thickness | 12 weeks | 0.9 (0.66) | 0.8 (0.63) | 0.9 (0.69) | 1.3 (0.85) |
| | Change | −1.0 (0.76) | −1.0 (0.79) | −0.9 (0.80) | −0.6 (0.82) |
| | LS Mean (SE) | −0.98 (0.05)* | −1.03 (0.05)* | −0.94 (0.05)* | −0.61 (0.05) |
| Vaginal | Baseline | 1.8 (0.68) | 1.7 (0.66) | 1.7 (0.63) | 1.8 (0.63) |
| secretions | 12 weeks | 0.8 (0.69) | 0.6 (0.67) | 0.7 (0.71) | 1.1 (0.84) |
| | Change | −1.0 (0.82) | −1.0 (0.86) | −1.0 (0.85) | −0.7 (0.79) |
| | LS Mean (SE) | −1.01 (0.05)* | −1.06 (0.05)* | −1.04 (0.05)* | −0.64 (0.05) |

Data is mean (SD) unless otherwise noted; *MMRM p < 0.0001 vs placebo.

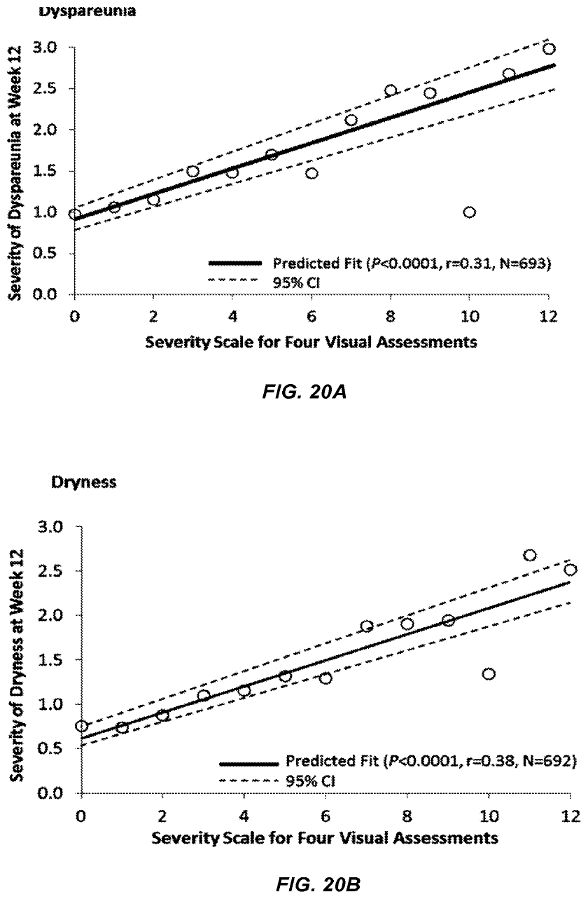

A direct correlation was observed between the total sum of the individual visual examination score and severity of dyspareunia (r=0.31; P<0.0001) as well as the severity of vaginal dryness (r=0.38; P<0.0001) at 12 weeks when all subjects were analyzed independent of treatment. See, FIG. 20A and FIG. 20B. Interestingly, women treated with placebo also showed some improvements in their scores at week 2, but while women treated with TX-004HR showed continued improvements through 12 weeks of treatment, such continued improvements were not observed to the same extent with the placebo. Three possible explanations for the improvements observed with the placebo include the potential lubricating effect of the excipient Miglyol, a fractionated coconut oil contained in all softgel capsules, improved appearance based on vaginal lubrication caused by increased sexual activity and/or bias on the part of the physicians performing the examinations as they may anticipate improvement. Nevertheless, TX-0041HR still significantly improved evaluated signs and symptoms of VVA better than placebo.

Since visual inspection of the vagina with the 4-point assessment tool positively correlated with dyspareunia and vaginal dryness in this study, this tool may help healthcare professionals diagnose VVA and assess its treatment, and provide a vehicle for health care professionals to initiation discussion with their patients about a sensitive topic. Several large-scale studies have shown that it is difficult for patients to discuss vulvovaginal health openly with their health care professionals because they are either embarrassed, uninformed about VVA and its treatments, or believe that the topic is not appropriate for discussion. Therefore, of the 50% of postmenopausal women who have symptoms of VVA, far fewer seek treatment. Visual examination of the vagina may help practitioners identify women at risk of dyspareunia and vaginal dryness, and allow them to proactively engage women in conversations about VVA symptoms such as dyspareunia and dryness and discuss available treatment options.

### Example 12: Pharmacokinetics Results in Randomized, Double-Blind, Placebo-Controlled Multicenter Study

While some approved local estrogens effectively treat VVA, systemic estradiol may increase with local administration. TX-004HR is a new low-dose vaginal softgel capsule containing solubilized natural estradiol designed to provide excellent efficacy with negligible systemic absorption. Up to three times lower systemic estrogen levels were previously reported with TX-004HR vs an approved low-dose vaginal estradiol tablet. The present studies show that VVA efficacy can be achieved with negligible systemic absorption as measured by PK in postmenopausal women with moderate-to-severe dyspareunia.

The terms “minimal systemic effect,” “low systemic absorption,” and “negligible systemic absorption,” as used herein, mean that the disclosed formulations and methods result in low to minimal absorption of estradiol in women, especially women with VVA and/or dyspareunia. In fact, it has surprisingly been found that the disclosed formulations and methods result in negligible to very low systemic absorption of estradiol, which remains in the postmenopausal range. The finding is borne out by the examples provided herein that demonstrate that the Cₘₐₓ and AUC levels of estradiol relative to placebo were not statistically differentiable, which indicates that the formulations disclosed herein have a negligible systemic effect. As such, the disclosed formulations and methods advantageously provide local benefits in patients with VVA and/or dyspareunia (i.e., the disclosed formulations are extremely effective in increasing the superficial cells, decreasing parabasal cells, and decreasing pH) without increasing systemic levels.

A PK substudy was part of a large, multicenter, double-blind, randomized, placebo-controlled phase 3 trial evaluating the efficacy and safety of TX-004HR (4 μg, 10 μg, and 25 μg) compared with placebo for treating postmenopausal moderate-to-severe dyspareunia. Women received TX-004HR or placebo once daily for 2 weeks then twice weekly for 10 wks.

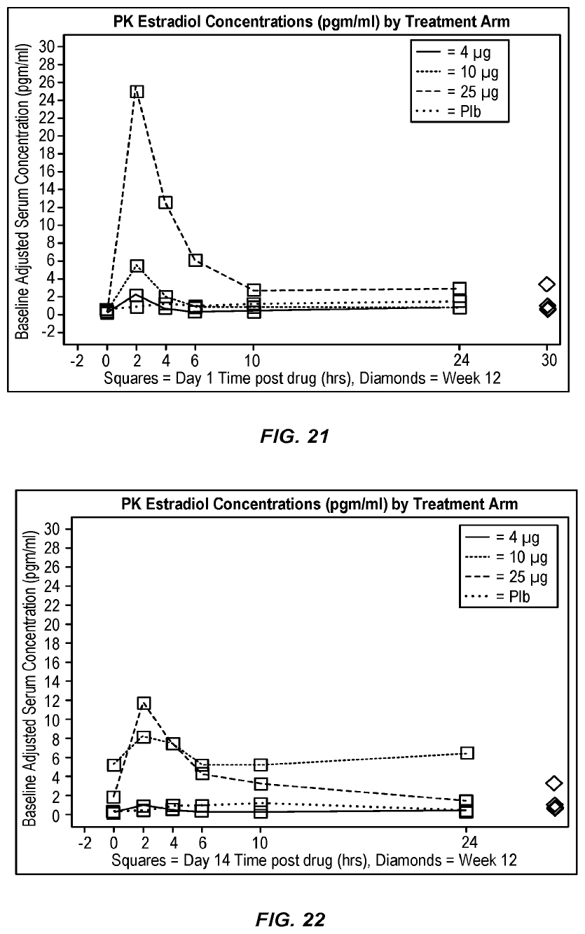

In this study, the systemic exposure to estradiol following once daily intravaginal administration of estradiol 25 μg, 10 μg, 4 μg, and placebo were investigated on days 1, 14, and 84 as described herein. Descriptive statistics of the plasma estradiol concentrations taken at each sampling time and the observed Cₘₐₓ values were recorded, as shown in the tables below and FIG. 21 and FIG. 22, for estradiol, estrone, and estrone conjugates for all three doses. Serum estradiol, estrone, estrone conjugates, and sex hormone binding globulin were measured.

For PK, serum was sampled pre-dose and at 2, 4, 6, 10, and 24 h post-dose on days 1 and 14 for estradiol, estrone (E1), and estrone conjugates (E1Cs). Baseline-adjusted results are shown here; unadjusted data will be presented. Efficacy endpoints were change from baseline to week 12 for vaginal superficial cells (%), vaginal parabasal cells (%), vaginal pH, and severity of dyspareunia. Secondary endpoints were severity of dryness and itching/irritation. Blood chemistry was tested at week 12.

The substudy randomized 72 women (mean age 59 y) at 11 centers. Mean area under the concentration-time curve (AUC) and average concentration (C_{avg}) for estradiol were not significantly different vs placebo with 4 μg and 10 μg TX-004HR, but were significantly higher with 25 μg at day 1 (AUC 130 vs 13.8 h*pg/mL and Cavg 5.4 vs 0.4 pg/mL) and day 14 (AUC 84.6 vs 7.1 h*pg/mL and Cavg 3.5 vs −0.2 pg/mL).

Mean estradiol peak concentration (Cₘₐₓ) was not significantly different with 4 μg (day 1: 2.6 pg/mL; day 14: 1.3 pg/mL) vs placebo (day 1: 2.1 pg/mL; day 14: 1.0 pg/mL), and although significant, was negligible with 10 μg (day 1: 6.0 pg/mL; day 14: 3.0 pg/mL) and very low for 25 μg (day 1: 26.2 pg/mL; day 14: 12.0 pg/mL).

E1 and E1Cs AUC, C_{avg}, Cₘₐₓ, Cₘᵢₙ did not differ vs placebo, except for E1Cs on day 1 when AUC was significantly higher with 25 μg (2454 vs 83.0 h*pg/mL), Cₘₐₓ with 10 μg and 25 μg (90.2 and 198.6 pg/mL, respectively vs 27.1 pg/mL), and C_{avg} with 10 μg (8.0 vs-33.7 pg/mL).

In the overall study TX004-HR showed robust efficacy for symptoms of dyspareunia, vaginal dryness and irritation at 12 weeks with all 3 doses compared with placebo.

Vaginal TX-004HR resulted in negligible to very low systemic absorption of estradiol, which remained in the postmenopausal range. TX-004HR improved the signs and symptoms of VVA. This study supports local benefits of estradiol without increasing systemic exposure.

The pharmacokinetic data for estradiol demonstrates the rapid absorption of the formulations disclosed herein for all three doses. Surprisingly, while the pharmacokinetic data was extremely low for all three doses, each dose was extremely effective in increasing the superficial cells, decreasing parabasal cells, and decreasing pH.

The pharmaceutical compositions disclosed herein provide an improved safety profile over other options for treating VVA. The combination of low systemic estradiol, while retaining efficacy was a surprising result for all three doses.

### Estradiol Concentration

TABLE 58
Pharmacokinetics Estradiol Baseline (pg/mL)

| | 4 μg | 10 μg | 25 μg | Placebo |
|---|---|---|---|---|
| Baseline | 4.7(4.41) | 5(3.52) | 3.6(1.86) | 4.6(2.56) |

TABLE 59
Pharmacokinetics Estradiol Day 1 (pg/mL)

| | 4 μg | 10 μg | 25 μg | Placebo |
|---|---|---|---|---|
| Predose | 3.1(1.56) | 4.9(3.47) | 3.6(1.81) | 4.1(2.45) |
| 2 hour | 6.1(2.3) | 10.4(4.89) | 28.7(17.91) | 4.8(3.33) |
| 4 hour | 4.3(1.68) | 6.7(3.59) | 16.1(14.75) | 5(3.59) |
| 6 hour | 3.7(1.96) | 5.7(3.16) | 9.7(6.86) | 4.8(3.53) |
| 10 hour | 3.7(1.47) | 5.5(2.92) | 6.2(2.37) | 5.2(3.61) |
| 24 hour | 4.2(2.02) | 5.4(4.44) | 6.2(8.43) | 5.1(4.42) |

TABLE 60
Pharmacokinetics Estradiol Day 14 (pg/mL)

| | 4 μg | 10 μg | 25 μg | Placebo |
|---|---|---|---|---|
| Predose | 3.5(1.63) | 3.8(2.56) | 5.2(2.89) | 4.2(3.07) |
| 2 hour | 4.3(2.01) | 6.3(2.29) | 15.3(7.72) | 4.2(2.44) |
| 4 hour | 4(1.7) | 5.9(2.55) | 11(4.86) | 4.7(3.2) |
| 6 hour | 3.9(1.92) | 5.1(2.32) | 7.9(3.35) | 4.7(2.97) |
| 10 hour | 3.8(2.12) | 5(3) | 6.8(3.76) | 5.1(3.53) |
| 24 hour | 3.6(1.89) | 3.7(2.05) | 4.9(4.35) | 3.9(2.43) |

TABLE 61
Pharmacokinetics Estradiol End of Study (pg/mL)

| | 4 μg | 10 μg | 25 μg | Placebo |
|---|---|---|---|---|
| Post Dosing | 4.3(2.69) | 4.8(2.57) | 6.7(11.51) | 4.4(2.6) |

TABLE 62
Estradiol Area Under the Curve (0-24 hours) (h*pg/mL)

| | 4 μg | 10 μg | 25 μg | Placebo |
|---|---|---|---|---|
| Day 1 | 91.7(37.86) | 138.2(75.22) | 217.4(99.02) | 116.6(77.3) |
| Day 14 | 87.2(42.77) | 110.1(54.57) | 171.6(80.13) | 104.2(66.39) |

TABLE 63
Estradiol Area Under the Curve (0-24 hours)
(Baseline Adjusted) (h*pg/mL)

| | 4 μg | 10 μg | 25 μg | Placebo |
|---|---|---|---|---|
| Day 1 | 12(13.89) | 21.9(19.16) | 130.4(111.95) | 13.8(28.86) |
| Day 14 | 7.2(12.08) | 13.7(18.77) | 84.6(62.7) | 7.1(20.28) |

TABLE 64
Estradiol Area Under the Curve (0-24 hours) P-values
Pairwise Test vs. 4 μg

| | 10 μg | 25 μg |
|---|---|---|
| Day 1 | 0.0242 | <0.0001 |
| Day 14 | 0.1777 | 0.0005 |

TABLE 65
Estradiol Area Under the Curve (0-24 hours) P-values
Pairwise Test vs. Placebo

| | 4 μg | 10 μg | 25 μg |
|---|---|---|---|
| Day 1 | 0.2292 | 0.4028 | 0.0021 |
| Day 14 | 0.3829 | 0.7724 | 0.0108 |

TABLE 66
Estradiol Area Under the Curve (0-24 hours) P-values
Pairwise Test vs. 4 μg (Baseline Adjusted)

| | 10 μg | 25 μg |
|---|---|---|
| Day 1 | 0.082 | 0.0001 |
| Day 14 | 0.2373 | 0.0001 |

TABLE 67
Estradiol Area Under the Curve (0-24 hours) P-values
Pairwise Test vs. Placebo (Baseline Adjusted)

| | 4 μg | 10 μg | 25 μg |
|---|---|---|---|
| Day 1 | 0.8134 | 0.3238 | 0.0002 |
| Day 14 | 0.979 | 0.3235 | <0.0001 |

TABLE 68
Estradiol Area Under the Curve (0-24 hours) Ratio (Day 14) of Day 14 to Day 1

| | 4 μg | 10 μg | 25 μg | Placebo |
|---|---|---|---|---|
| AUC Ratio of Day 14 | 0.971(0.2358) | 0.876(0.1937) | 0.955(0.6633) | 0.949(0.225) |
| to Day 1 | | | | |
| Pairwise test vs 4 μg | — | 0.2022 | 0.9246 | — |
| Pairwise test vs | 0.7859 | 0.3101 | 0.9748 | — |
| Placebo | | | | |

### Estradiol Cₘₐₓ

TABLE 69
Cₘₐₓ (pg/mL)

| | 4 μg | | 10 μg | | 25 μg | | Placebo | |
|---|---|---|---|---|---|---|---|---|
| Day 1 | 6.5 | (2.13) | 10.9 | (5) | 29.8 | (17.51) | 6.6 | (4.85) |
| Day 14 | 4.8 | (2.31) | 7.3 | (2.36) | 15.7 | (7.61) | 5.5 | (3.43) |

TABLE 70
Cₘₐₓ (Baseline Adjusted) (pg/mL)

| | 4 μg | | 10 μg | | 25 μg | | Placebo | |
|---|---|---|---|---|---|---|---|---|
| Day 1 | 2.6 | (2.17) | 6 | (4.44) | 26.2 | (18.19) | 2.1 | (3.48) |
| Day 14 | 1.3 | (1.08) | 3 | (1.73) | 12 | (7.32) | 1 | (1.81) |

TABLE 71
Cₘₐₓ P-values Pairwise Test vs. 4 μg

| | 10 μg | 25 μg |
|---|---|---|
| Day 1 | 0.0013 | <0.0001 |
| Day 14 | 0.0033 | <0.0001 |

TABLE 72
Cₘₐₓ P-values Pairwise Test vs. Placebo

| | 4 μg | 10 μg | 25 μg |
|---|---|---|---|
| Day 1 | 0.9586 | 0.0116 | <0.0001 |
| Day 14 | 0.5174 | 0.0702 | <0.0001 |

TABLE 73
Cₘₐₓ P-values Pairwise Test vs.
4 μg (Baseline Adjusted)

| | 10 μg | 25 μg |
|---|---|---|
| Day 1 | 0.0055 | <0.0001 |
| Day 14 | 0.002 | <0.0001 |

TABLE 74
Cₘₐₓ P-values Pairwise Test vs.
Placebo (Baseline Adjusted)

| | 4 μg | 10 μg | 25 μg |
|---|---|---|---|
| Day 1 | 0.6074 | 0.0059 | <0.0001 |
| Day 14 | 0.5088 | 0.0022 | <0.0001 |

TABLE 75
Cₘₐₓ Ratio (Day 14) of Day 14 to Day 1

| | 4 μg | 10 μg | 25 μg | Placebo |
|---|---|---|---|---|
| Cₘₐₓ Ratio | 0.77 | 0.804 | 0.929 | 0.933 |
| of Day 14 to | (0.2633) | (0.3245) | (1.5011) | (0.2406) |
| Day 1 | | | | |
| Pairwise | — | 0.7399 | 0.6702 | — |
| test vs | | | | |
| Pairwise | 0.0702 | 0.1946 | 0.9931 | — |
| test vs | | | | |
| Placebo | | | | |

### Estradiol C_{avg}

TABLE 76
C_{avg} (pg/mL)

| | 4 μg | | 10 μg | | 25 μg | | Placebo | |
|---|---|---|---|---|---|---|---|---|
| Day 1 | 3.9 | (1.46) | 5.8 | (3.13) | 9.1 | (4.13) | 4.9 | (3.22) |
| Day 14 | 3.6 | (1.78) | 4.6 | (2.27) | 7.1 | (3.34) | 4.3 | (2.77) |

TABLE 77
C_{avg} (Baseline Adjusted) (pg/mL)

| | 4 μg | | 10 μg | | 25 μg | | Placebo | |
|---|---|---|---|---|---|---|---|---|
| Day 1 | 0 | (1.93) | 0.8 | (0.95) | 5.4 | (4.66) | 0.4 | (1.35) |
| Day 14 | 0.1 | (0.68) | 0.2 | (1.22) | 3.5 | (2.61) | −0.2 | (1.28) |

TABLE 78
C_{avg} P-values Pairwise Test vs. 4 μg

| | 10 μg | 25 μg |
|---|---|---|
| Day 1 | 0.0294 | <0.0001 |
| Day 14 | 0.1777 | 0.0005 |

TABLE 79
C_{avg} P-values Pairwise Test vs. Placebo

| | 4 μg | 10 μg | 25 μg |
|---|---|---|---|
| Day 1 | 0.267 | 0.4028 | 0.0021 |
| Day 14 | 0.3829 | 0.7724 | 0.0108 |

TABLE 80
C_{avg} P-values Pairwise Test vs.
4 μg (Baseline Adjusted)

| | 10 μg | 25 μg |
|---|---|---|
| Day 1 | 0.1076 | 0.0001 |
| Day 14 | 0.7759 | <0.0001 |

TABLE 81
C_{avg} P-values Pairwise Test vs.
Placebo (Baseline Adjusted)

| | 4 μg | 10 μg | 25 μg |
|---|---|---|---|
| Day 1 | 0.5126 | 0.2564 | 0.0001 |
| Day 14 | 0.4098 | 0.3629 | <0.0001 |

TABLE 82
C_{avg} Ratio (Day 14) of Day 14 to Day 1

| | 4 μg | 10 μg | 25 μg | Placebo |
|---|---|---|---|---|
| C_{avg} Ratio | 0.77 (0.2633) | 0.804 (0.3245) | 0.929 (1.5011) | 0.933 (0.2406) |
| of Day 14 | | | | |
| to Day 1 | | | | |
| Pairwise | — | 0.7399 | 0.6702 | — |
| test vs | | | | |
| Pairwise | 0.0702 | 0.1946 | 0.9931 | — |
| test vs | | | | |
| Placebo | | | | |

### Estradiol Tₘₐₓ

TABLE 83
Tₘₐₓ (h)

| | 4 μg | | 10 μg | | 25 μg | | Placebo | |
|---|---|---|---|---|---|---|---|---|
| Day 1 | 7 | (9.36) | 6.1 | (8.04) | 4.6 | (7.09) | 8.6 | (6.74) |
| Day 14 | 9.3 | (8.86) | 4 | (2.57) | 2.7 | (1.94) | 7.2 | (3) |

TABLE 84
Tₘₐₓ P-values Pairwise Test vs. 4 μg

| | 10 μg | 25 μg |
|---|---|---|
| Day 1 | 0.7566 | 0.3834 |
| Day 14 | 0.0206 | 0.004 |

TABLE 85
Tₘₐₓ P-values Pairwise Test vs. Placebo

| | 4 μg | 10 μg | 25 μg |
|---|---|---|---|
| Day 1 | 0.5705 | 0.3255 | 0.0943 |
| Day 14 | 0.3576 | 0.0019 | <0.0001 |

### Estrone Concentration

TABLE 86
Pharmacokinetics Estrone Baseline (pg/mL)

| | 4 μg | | 10 μg | | 25 μg | | Placebo | |
|---|---|---|---|---|---|---|---|---|
| Baseline | 15.9 | (6.02) | 19.7 | (9.18) | 16.3 | (7.71) | 20.4 | (9.67) |

TABLE 87
Pharmacokinetics Estrone Day 1 (pg/mL)

| | 4 μg | | 10 μg | | 25 μg | | Placebo | |
|---|---|---|---|---|---|---|---|---|
| Predose | 14.7 | (4.44) | 21 | (8.51) | 17.2 | (8.5) | 18.3 | (8.54) |
| 2 hour | 13.3 | (4.52) | 20 | (8.53) | 18.9 | (6.7) | 18.9 | (11.25) |
| 4 hour | 13 | (4.68) | 19.3 | (7.4) | 19.4 | (7.06) | 19.9 | (13.87) |
| 6 hour | 13.9 | (6.04) | 19.6 | (8.89) | 19.1 | (8.1) | 19 | (11.69) |
| 10 hour | 13.4 | (4.94) | 19.7 | (8.53) | 18.8 | (7.18) | 19.3 | (11.65) |
| 24 hour | 14.3 | (5.92) | 21.2 | (9.89) | 16.6 | (6.06) | 22.9 | (17.18) |

TABLE 88
Pharmacokinetics Estrone Day 14 (pg/mL)

| | 4 μg | | 10 μg | | 25 μg | | Placebo | |
|---|---|---|---|---|---|---|---|---|
| Predose | 15.8 | (5.15) | 21.7 | (14.25) | 18.6 | (8.49) | 18.7 | (9.38) |
| 2 hour | 13.6 | (5.3) | 19.7 | (10.2) | 19.8 | (9.08) | 17.3 | (7.99) |
| 4 hour | 14 | (5.25) | 21 | (13.46) | 19.9 | (7.26) | 20.4 | (11.41) |
| 6 hour | 14 | (5.11) | 20.7 | (10.4) | 19.3 | (6.47) | 16.1 | (7.54) |
| 10 hour | 14.2 | (5.51) | 20.1 | (11.93) | 19.3 | (8.24) | 19 | (8.17) |
| 24 hour | 14.5 | (4.69) | 20.1 | (9.34) | 16.7 | (6.09) | 18.9 | (8.24) |

TABLE 89
Pharmacokinetics Estrone End of Study (pg/mL)

| | 4 μg | 10 μg | 25 μg | Placebo |
|---|---|---|---|---|
| Post Dosing | 4.328 | 4.643 | 6.652 | 4.363 |
| | (2.7619) | (2.5807) | (11.508) | (2.5982) |

### Estrone Area Under the Curve (0-24 Hours)

TABLE 90
Estrone Area Under the Curve (0-24 hours) (h*pg/mL)

| | 4 μg | | 10 μg | | 25 μg | | Placebo | |
|---|---|---|---|---|---|---|---|---|
| Day 1 | 290.2 | (123.67) | 462.7 | (195.64) | 419.1 | (147.85) | 467.9 | (278.78) |
| Day 14 | 326.6 | (114.09) | 464.1 | (243.92) | 428.7 | (161.75) | 426.8 | (180.67) |

TABLE 91
Estrone Area Under the Curve (0-24 hours) (Baseline Adjusted) (h*pg/mL)

| | 4 μg | | 10 μg | | 25 μg | | Placebo | |
|---|---|---|---|---|---|---|---|---|
| Day 1 | 7.2 | (20.91) | 10.9 | (24.55) | 44.3 | (54.27) | 43.5 | (97.41) |
| Day 14 | 15 | (41.53) | 43.2 | (84.87) | 55.6 | (78.06) | 17.4 | (45.27) |

TABLE 92
Estrone Area Under the Curve
(0-24 hours) P-values Pairwise
Test vs. 4 μg

| | 10 μg | 25 μg |
|---|---|---|
| Day 1 | 0.003 | 0.0076 |
| Day 14 | 0.042 | 0.0393 |

TABLE 93
Estrone Area Under the Curve (0-24 hours)
P-values Pairwise Test vs. Placebo

| | 4 μg | 10 μg | 25 μg |
|---|---|---|---|
| Day 1 | 0.0193 | 0.9487 | 0.519 |
| Day 14 | 0.0621 | 0.6117 | 0.9738 |

TABLE 94
Estrone Area Under the Curve
(0-24 hours) P-values
Pairwise Test vs. 4 μg
(Baseline Adjusted)

| | 10 μg | 25 μg |
|---|---|---|
| Day 1 | 0.6195 | 0.0104 |
| Day 14 | 0.2251 | 0.0658 |

TABLE 95
Estrone Area Under the Curve (0-24 hours)
P-values Pairwise Test vs. Placebo
(Baseline Adjusted)

| | 4 μg | 10 μg | 25 μg |
|---|---|---|---|
| Day 1 | 0.1311 | 0.167 | 0.9761 |
| Day 14 | 0.8721 | 0.2746 | 0.0886 |

TABLE 96
Estrone Area Under the Curve (0-24 hours)
Ratio (Day 14) of Day 14 to Day 1

| | 4 μg | 10 μg | 25 μg | Placebo |
|---|---|---|---|---|
| AUC Ratio of | 1.234 | 1.023 | 1.039 | 1.006 |
| Day 14 to Day 1 | (0.5824) | (0.2675) | (0.1941) | (0.2316) |
| Pairwise test vs | — | 0.1722 | 0.1866 | — |
| Pairwise test vs | 0.1432 | 0.848 | 0.6544 | — |
| Placebo | | | | |

### Estrone Cₘₐₓ

TABLE 97
Cₘₐₓ (pg/mL)

| | 4 μg | | 10 μg | | 25 μg | | Placebo | |
|---|---|---|---|---|---|---|---|---|
| Day 1 | 15.7 | (6.07) | 23.5 | (9.87) | 21.9 | (7.73) | 25.7 | (18.43) |
| Day 14 | 16 | (5.5) | 23.9 | (13.45) | 22.4 | (8.95) | 22.8 | (10.89) |

TABLE 98
Cₘₐₓ (Baseline Adjusted) (pg/mL)

| | 4 μg | | 10 μg | | 25 μg | | Placebo | |
|---|---|---|---|---|---|---|---|---|
| Day 1 | 0.4 | (3.05) | 3.2 | (2.99) | 5.1 | (4.78) | 6.3 | (12.81) |
| Day 14 | 0.6 | (3.49) | 3.7 | (8.79) | 5.6 | (4.81) | 3.4 | (5.69) |

TABLE 99
Cₘₐₓ P-values Pairwise Test vs. 4 μg

| | 10 μg | 25 μg |
|---|---|---|
| Day 1 | 0.007 | 0.0126 |
| Day 14 | 0.0301 | 0.0163 |

TABLE 100
Cₘₐₓ P-values Pairwise Test vs. Placebo

| | 4 μg | 10 μg | 25 μg |
|---|---|---|---|
| Day 1 | 0.0373 | 0.6567 | 0.4223 |
| Day 14 | 0.0275 | 0.7878 | 0.8979 |

TABLE 101
Cₘₐₓ P-values Pairwise Test vs. 4 μg (Baseline Adjusted)

| | 10 μg | 25 μg |
|---|---|---|
| Day 1 | 0.0087 | 0.0013 |
| Day 14 | 0.1975 | 0.0014 |

TABLE 102
Cₘₐₓ P-values Pairwise Test vs. Placebo (Baseline Adjusted)

| | 4 μg | 10 μg | 25 μg |
|---|---|---|---|
| Day 1 | 0.0659 | 0.3046 | 0.71 |
| Day 14 | 0.0938 | 0.933 | 0.2249 |

TABLE 103
Cₘₐₓ Ratio (Day 14) of Day 14 to Day 1

| | 4 μg | 10 μg | 25 μg | Placebo |
|---|---|---|---|---|
| Cₘₐₓ Ratio of | 1.029 | 1.042 | 1.041 | 1.039 |
| Day 14 to Day 1 | (0.2346) | (0.3436) | (0.2179) | (0.2916) |
| Pairwise test vs | — | 0.9035 | 0.8835 | — |
| Pairwise test vs | 0.9188 | 0.9788 | 0.982 | — |
| Placebo | | | | |

### Estrone C_{avg}

TABLE 104
C_{avg} (pg/mL)

| | 4 μg | 10 μg | 25 μg | Placebo |
|---|---|---|---|---|
| Day 1 | 13(4.72) | 19.3(8.15) | 17.5(6.16) | 19.5(11.62) |
| Day 14 | 13.6(4.75) | 19.3(10.16) | 17.9(6.74) | 17.8(7.53) |

TABLE 105
C_{avg} (Baseline Adjusted) (pg/mL)

| | 4 μg | 10 μg | 25 μg | Placebo |
|---|---|---|---|---|
| Day 1 | −2.3(2.26) | −1.1(2.66) | 0.7(3.73) | 0.1(5.03) |
| Day 14 | −1.7(3.25) | −0.9(5.91) | 1.1(4.81) | −1.6(3.8) |

TABLE 106
C_{avg} P-values Pairwise Test vs. 4 μg

| | 10 μg | 25 μg |
|---|---|---|
| Day 1 | 0.0075 | 0.0207 |
| Day 14 | 0.042 | 0.0393 |

TABLE 107
C_{avg} P-values Pairwise Test vs. Placebo

| | 4 μg | 10 μg | 25 μg |
|---|---|---|---|
| Day 1 | 0.0363 | 0.9487 | 0.519 |
| Day 14 | 0.0621 | 0.6117 | 0.9738 |

TABLE 108
C_{avg} P-values Pairwise Test vs. 4 μg (Baseline Adjusted)

| | 10 μg | 25 μg |
|---|---|---|
| Day 1 | 0.1345 | 0.0057 |
| Day 14 | 0.6351 | 0.0495 |

TABLE 109
C_{avg} P-values Pairwise Test vs. Placebo (Baseline Adjusted)

| | 4 μg | 10 μg | 25 μg |
|---|---|---|---|
| Day 1 | 0.0712 | 0.3751 | 0.691 |
| Day 14 | 0.912 | 0.7058 | 0.0742 |

TABLE 110
C_{avg} Ratio (Day 14) of Day 14 to Day 1

| | 4 μg | 10 μg | 25 μg | Placebo |
|---|---|---|---|---|
| C_{avg} Ratio of | 1.029 | 1.042 | 1.041 | 1.039 |
| Day 14 to Day 1 | (0.2346) | (0.3436) | (0.2179) | (0.2916) |
| Pairwise test vs | — | 0.9035 | 0.8835 | — |
| Pairwise test vs | 0.9188 | 0.9788 | 0.982 | — |
| Placebo | | | | |

### Estrone Tₘₐₓ

TABLE 111
Tₘₐₓ (h)

| | 4 μg | 10 μg | 25 μg | Placebo |
|---|---|---|---|---|
| Day 1 | 14.1(9.37) | 11.9(9.76) | 9.1(7.43) | 12.1(9.39) |
| Day 14 | 10.9(9.03) | 10.4(8.93) | 6.3(6.9) | 12.2(9.24) |

TABLE 112
Tₘₐₓ P-values Pairwise Test vs. 4 μg

| | 10 μg | 25 μg |
|---|---|---|
| Day 1 | 0.4862 | 0.0849 |
| Day 14 | 0.8711 | 0.0982 |

TABLE 113
Tₘₐₓ P-values Pairwise Test vs. Placebo

| | 4 μg | 10 μg | 25 μg |
|---|---|---|---|
| Day 1 | 0.5341 | 0.9449 | 0.2997 |
| Day 14 | 0.6824 | 0.5639 | 0.0391 |

### Estrone Conjugates

TABLE 114
Pharmacokinetics Estrone Conjugates Baseline (pg/mL)

| | 4 μg | | 10 μg | | 25 μg | | Placebo | |
|---|---|---|---|---|---|---|---|---|
| Baseline | 250.3 | (162.91) | 259.7 | (208.51) | 374.4 | (586.45) | 280.7 | (171.26) |

TABLE 115
Pharmacokinetics Estrone Conjugates Day 1 (pg/mL)

| | 4 μg | | 10 μg | | 25 μg | | Placebo | |
|---|---|---|---|---|---|---|---|---|
| Predose | 225.1 | (215.01) | 218.6 | (147.84) | 312.4 | (410.38) | 271.2 | (153.33) |
| 2 hour | 206.8 | (163.2) | 273.1 | (176.59) | 396.6 | (408.16) | 223.4 | (162.11) |
| 4 hour | 241.7 | (176.87) | 267.2 | (161.79) | 413.3 | (343.25) | 241.8 | (139.77) |
| 6 hour | 240.6 | (181.14) | 266 | (184.92) | 477.8 | (472.66) | 265 | (154.01) |
| 10 hour | 223 | (150.42) | 243.5 | (173.71) | 436.4 | (461) | 258 | (133.21) |
| 24 hour | 229.4 | (186.79) | 268.4 | (221.29) | 306.4 | (322.91) | 268.8 | (153.22) |

TABLE 116
Pharmacokinetics Estrone Conjugates Day 14 (pg/mL)

| | 4 μg | | 10 μg | | 25 μg | | Placebo | |
|---|---|---|---|---|---|---|---|---|
| Predose | 212.7 | (140.19) | 319.1 | (326.71) | 411.1 | (624.14) | 256.1 | (133.07) |
| 2 hour | 212.4 | (145.02) | 420.4 | (560.53) | 434.3 | (491.31) | 285.6 | (158.61) |
| 4 hour | 240.2 | (155.7) | 429.3 | (506.01) | 505.1 | (618.47) | 273.1 | (148.76) |
| 6 hour | 225.8 | (164.76) | 359.2 | (346.26) | 483.8 | (515.95) | 267.7 | (181.53) |
| 10 hour | 238.3 | (152.45) | 417.6 | (517.51) | 492.5 | (598.16) | 306.9 | (178.68) |
| 24 hour | 206.4 | (154.26) | 349 | (345.91) | 309.6 | (380.88) | 240.1 | (115.84) |

TABLE 117
Pharmacokinetics Estrone Conjugates End of Study (pg/mL)

| | 4 μg | | 10 μg | | 25 μg | | Placebo | |
|---|---|---|---|---|---|---|---|---|
| Post Dosing | 237.4 | (151.19) | 221.7 | (188.05) | 499.7 | (1089.67) | 250 | (148.72) |

### Estrone Conjugates Area Under the Curve (0-24 Hours)

TABLE 118
Estrone Conjugates Area Under the Curve (0-24 hours) (h*pg/mL)

| | 4 μg | | 10 μg | | 25 μg | | Placebo | |
|---|---|---|---|---|---|---|---|---|
| Day 1 | 5077.5 | (3798.39) | 5931.9 | (4209.95) | 9126 | (9186.37) | 5637.9 | (3151.49) |
| Day 14 | 5172.9 | (3382.89) | 8978 | (9811.23) | 9930.2 | (11711.99) | 6275.2 | (3397.54) |

TABLE 119
Estrone Conjugates Area Under the Curve (0-24 hours)
(Baseline Adjusted) (h*pg/mL)

| | 4 μg | | 10 μg | | 25 μg | | Placebo | |
|---|---|---|---|---|---|---|---|---|
| Day 1 | 375.5 | (843.98) | 422.4 | (473.83) | 2454.3 | (2600.25) | 83 | (229.06) |
| Day 14 | 660.5 | (1230.69) | 3767.2 | (7671.38) | 3059 | (4792.46) | 665.4 | (1552.19) |

TABLE 120
Estrone Conjugates Area Under the Curve (0-24 hours)
P-values Pairwise Test vs. 4 μg

| | 10 μg | 25 μg |
|---|---|---|
| Day 1 | 0.5219 | 0.0931 |
| Day 14 | 0.1392 | 0.1166 |

TABLE 121
Estrone Conjugates Area Under the Curve (0-24 hours)
P-values Pairwise Test vs. Placebo

| | 4 μg | 10 μg | 25 μg |
|---|---|---|---|
| Day 1 | 0.639 | 0.8157 | 0.1472 |
| Day 14 | 0.3503 | 0.2898 | 0.2246 |

TABLE 122
Estrone Conjugates Area Under the Curve
(0-24 hours) P-values Pairwise Test vs.
4 μg (Baseline Adjusted)

| | 10 μg | 25 μg |
|---|---|---|
| Day 1 | 0.8349 | 0.0028 |
| Day 14 | 0.1087 | 0.0537 |

TABLE 123
Estrone Conjugates Area Under the Curve
(0-24 hours) P-values Pairwise Test vs.
Placebo (Baseline Adjusted)

| | 4 μg | 10 μg | 25 μg |
|---|---|---|---|
| Day 1 | 0.1894 | 0.0134 | 0.001 |
| Day 14 | 0.992 | 0.1225 | 0.0654 |

TABLE 124
Estrone Conjugates Area Under the Curve (0-24 hours)
Ratio (Day 14) of Day 14 to Day 1

| | 4 μg | | 10 μg | | 25 μg | | Placebo | |
|---|---|---|---|---|---|---|---|---|
| AUC Ratio of | 1.115 | (0.4539) | 1.444 | (1.0121) | 1.107 | (0.3545) | 1.125 | (0.4522) |

| | | | | |
|---|---|---|---|---|
| Day 14 to Day 1 | | | | |
| Pairwise test vs | — | 0.2279 | 0.9587 | — |
| Pairwise test vs | 0.9459 | 0.2427 | 0.8975 | — |
| Placebo | | | | |

### Estrone Conjugates Cₘₐₓ

TABLE 125
Cₘₐₓ (pg/mL)

| | 4 μg | | 10 μg | | 25 μg | | Placebo | |
|---|---|---|---|---|---|---|---|---|
| Day 1 | 273.1 | (196.36) | 329.4 | (226.58) | 542.1 | (475.49) | 309.8 | (146.07) |
| Day 14 | 289 | (183.79) | 511.7 | (568.75) | 579.5 | (610.1) | 343.6 | (182.2) |

TABLE 126
Cₘₐₓ (Baseline Adjusted) (pg/mL)

| | 4 μg | | 10 μg | | 25 μg | | Placebo | |
|---|---|---|---|---|---|---|---|---|
| Day 1 | 35.4 | (89.09) | 90.2 | (65.2) | 198.6 | (301.53) | 27.1 | (49.69) |
| Day 14 | 48.2 | (132.61) | 277.8 | (493.64) | 236.1 | (372.42) | 67 | (121.81) |

TABLE 127
Cₘₐₓ P-values Pairwise Test vs. 4 μg

| | 10 μg | 25 μg |
|---|---|---|
| Day 1 | 0.4261 | 0.0333 |
| Day 14 | 0.1332 | 0.0685 |

TABLE 128
Cₘₐₓ P-values Pairwise Test vs. Placebo

| | 4 μg | 10 μg | 25 μg |
|---|---|---|---|
| Day 1 | 0.5369 | 0.7629 | 0.0625 |
| Day 14 | 0.3902 | 0.2533 | 0.1356 |

TABLE 129
Cₘₐₓ P-values Pairwise vs. 4 μg (Baseline Adjusted)

| | 10 μg | 25 μg |
|---|---|---|
| Day 1 | 0.039 | 0.0345 |
| Day 14 | 0.0726 | 0.0579 |

TABLE 130
Cₘₐₓ P-values Pairwise Test vs. Placebo (Baseline Adjusted)

| | 4 μg | 10 μg | 25 μg |
|---|---|---|---|
| Day 1 | 0.7444 | 0.0033 | 0.0318 |
| Day 14 | 0.6735 | 0.1065 | 0.0928 |

TABLE 131
Cₘₐₓ Ratio (Day 14) of Day 14 to Day 1

| | 4 μg | 10 μg | 25 μg | Placebo |
|---|---|---|---|---|
| Cₘₐₓ Ratio | 1.13 | 1.524 | 1.144 | 1.11 |
| of Day 14 | (0.4068) | (1.1682) | (0.4569) | (0.5404) |
| to Day 1 | | | | |
| Pairwise | — | 0.1969 | 0.9226 | — |
| test vs | | | | |
| Pairwise | 0.9043 | 0.1919 | 0.8406 | — |
| test vs | | | | |
| Placebo | | | | |

### Estrone Conjugates C_{avg}

TABLE 132
C_{avg} (pg/mL)

| | 4 μg | 10 μg | 25 μg | Placebo |
|---|---|---|---|---|
| Day 1 | 215.9 (154.77) | 247.2 (175.41) | 380.3 (382.77) | 244.6 (128.1) |
| Day 14 | 215.5 (140.95) | 374.1 (408.8) | 413.8 (488) | 261.5 (141.56) |

TABLE 133
C_{avg} (Baseline Adjusted) (pg/mL)

| | 4 μg | 10 μg | 25 μg | Placebo |
|---|---|---|---|---|
| Day 1 | −21.8 (88.41) | 8 (34.21) | 36.8 (291.72) | −33.7 (46.95) |
| Day 14 | −25.3 (120.69) | 140.2 (330.6) | 70.3 (300.36) | −7.9 (89.89) |

TABLE 134
C_{avg} P-values Pairwise Test vs. 4 μg

| | 10 μg | 25 μg |
|---|---|---|
| Day 1 | 0.5701 | 0.1004 |
| Day 14 | 0.1392 | 0.1166 |

TABLE 135
C_{avg} P-values Pairwise Test vs. Placebo

| | 4 μg | 10 μg | 25 μg |
|---|---|---|---|
| Day 1 | 0.5562 | 0.9602 | 0.1741 |
| Day 14 | 0.3503 | 0.2898 | 0.2246 |

TABLE 136
C_{avg} P-values Pairwise Test vs. 4 μg (Baseline Adjusted)

| | 10 μg | 25 μg |
|---|---|---|
| Day 1 | 0.1804 | 0.4201 |
| Day 14 | 0.0606 | 0.2305 |

TABLE 137
C_{avg} P-values Pairwise Test vs. Placebo (Baseline Adjusted)

| | 4 μg | 10 μg | 25 μg |
|---|---|---|---|
| Day 1 | 0.6353 | 0.0047 | 0.3473 |
| Day 14 | 0.6439 | 0.0928 | 0.3244 |

TABLE 138
C_{avg} Ratio (Day 14) of Day 14 to Day 1

| | 4 μg | 10 μg | 25 μg | Placebo |
|---|---|---|---|---|
| C_{avg} Ratio | 1.13 | 1.524 | 1.144 | 1.11 |
| of Day 14 | (0.4068) | (1.1682) | (0.4569) | (0.5404) |
| to Day 1 | | | | |
| Pairwise | — | 0.1969 | 0.9226 | — |
| test vs | | | | |
| Pairwise | 0.9043 | 0.1919 | 0.8406 | — |
| test vs | | | | |
| Placebo | | | | |

### Estrone Conjugates Tₘₐₓ

TABLE 139
Tₘₐₓ (h)

| | 4 μg | 10 μg | 25 μg | Placebo |
|---|---|---|---|---|
| Day 1 | 10.9 (8.66) | 9.2 (9.25) | 5.4 (2.64) | 13.1 (9.7) |
| Day 14 | 8.4 (7.79) | 9 (8.6) | 5.9 (2.87) | 8.1 (6.76) |

TABLE 140
Tₘₐₓ P-values Pairwise Test vs. 4 μg

| | 10 μg | 25 μg |
|---|---|---|
| Day 1 | 0.5609 | 0.0154 |
| Day 14 | 0.8173 | 0.2178 |

TABLE 141
Tmax P-values Pairwise Test vs. Placebo

| | 4 μg | 10 μg | 25 μg |
|---|---|---|---|
| Day 1 | 0.4893 | 0.2253 | 0.003 |
| Day 14 | 0.9256 | 0.739 | 0.2087 |

In the phase 3 trial, all doses of TX-004HIR compared with placebo (MITT n=747) significantly improved the 4 co-primary endpoints at week 2 through week 12, as well as the secondary endpoints of vaginal dryness by week 6 and vulvar and/or vaginal itching or irritation by week 12 (except 4 μg, p=0.0503), and was well-tolerated with no treatment-related serious AEs reported. The phase 3 PK study (n=72) showed no difference in systemic E2 levels for 4 μg and 10 μg TX-004HR vs placebo, as measured by AUC and C_{avg}. E2 AUC and C_{avg} with 25 μg TX-004HR was higher than placebo, but average concentrations remained within the normal postmenopausal range (Table 142). E2 levels at day 84 were similar to placebo indicating no systemic drug accumulation. SHBG concentrations did not change with treatment. The two phase 2 studies (n=36 for each) of TX-004HR 10 μg and 25 μg resulted in statistically significantly lower E2 absorption than an approved E2 tablet at identical doses, with 25 μg TX-004HR demonstrating AUC less than ⅓ that of the approved product (Table 143).

TABLE 142
Phase 3 study PK parameters for E2 (unadjusted mean ± SD).

| | | AUC₀₋₂₄ (pg*hr/mL) | | | C_{avg} (pg/mL) | | |
|---|---|---|---|---|---|---|---|
| Day | Dose (μg) | TX-004HR | Placebo | p-value | TX-004HR | Placebo | p-value |
| 1 | 4 | 91.7 ± 37.9 | 116.6 ± 77.3 | NS | 3.92 ± 1.46 | 4.86 ± 3.22 | NS |
| | 10 | 138.2 ± 75.2 | 116.6 ± 77.3 | NS | 5.76 ± 3.13 | 4.86 ± 3.22 | NS |
| | 25 | 217.4 ± 99.0 | 116.6 ± 77.3 | 0.0021 | 9.06 ± 4.13 | 4.86 ± 3.22 | 0.0021 |
| 14 | 4 | 87.2 ± 42.8 | 104.2 ± 66.4 | NS | 3.63 ± 1.78 | 4.34 ± 2.77 | NS |
| | 10 | 110.1 ± 54.6 | 104.2 ± 66.4 | NS | 4.59 ± 2.27 | 4.34 ± 2.77 | NS |
| | 25 | 171.6 ± 80.1 | 104.2 ± 66.4 | 0.0108 | 7.15 ± 3.34 | 4.34 ± 2.77 | 0.0108 |

TABLE 143
Phase 2 studies PK parameters for E2 (baseline adjusted geometric mean).

| | AUC₀₋₂₄ (pg*hr/mL) | | | Cₘₐₓ (pg/mL) | | |
|---|---|---|---|---|---|---|
| Dose (μg) | TX-004HR | Vaginal Tablet | p-value | TX-004HR | Vaginal Tablet | p-value |
| 10 | 49.62 | 132.92 | <0.0001 | 14.38 | 20.38 | 0.0194 |
| 25 | 89.21 | 292.06 | <0.0001 | 23.08 | 42.70 | <0.0001 |

With robust efficacy demonstrated as early as 2 weeks and up to 12 weeks at all 3 doses, TX-004HR 4 μg and 10 μg showed negligible systemic E2 absorption, while 25 μg resulted in very low systemic absorption of E2 in the phase 3 trial. TX-004HR 10 μg and 25 μg showed lower systemic E2 exposure than equivalent doses of an approved E2 tablet. The absence of clinically meaningful increases in E2 concentrations paired with data consistent with a lack of systemic effects (e.g., no increase in SHBG) shows that TX-004 HR delivers excellent efficacy with negligible to very low systemic exposure.

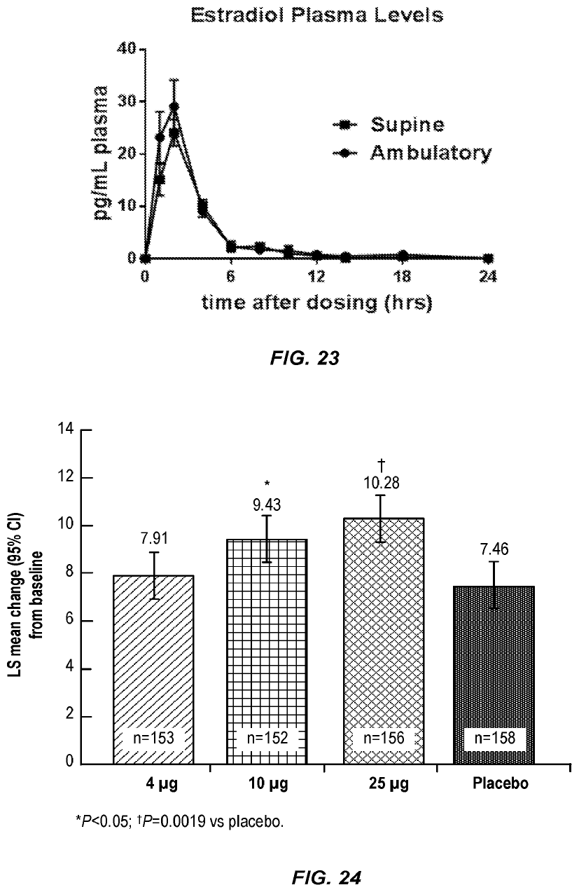

The impact of normal daily activities for 4 hours post dose was evaluated, in comparison with the impact of remaining in the supine position for 4 hours post dose on the pharmacokinetic (PK) profile of TX-004HR 25 mcg. In two studies, at the same site, the same sixteen healthy postmenopausal female subjects were fasted for at least 10 hours prior to dosing through 4 hours following dosing. Subjects received a 25 mcg dose of TX-004HR administered intravaginally by trained female study personnel. Following their first dose, the subjects were required to remain in a supine position for 4 hours following dosing. Following the second dose, after 5 minutes resting time, the subjects were instructed to be ambulatory in the clinic and refrain from reclining for the 4 hours following dosing. Blood samples were collected at pre-defined intervals up to 24 hours after dosing. Plasma samples were analyzed for estradiol using LC-MS/MS. See, e.g., FIG. 23. PK parameters were calculated on an individual and group mean basis with baseline correction.

The mean Cₘₐₓ and AUC₀₋₂₄ of estradiol was not significantly different with ambulation than with supination. On an individual subject basis, the majority showed similar Cₘₐₓ and AUC₀₋₂₄ levels with ambulation as with supination. There were no signs of posture having an effect on absorption rate as evidenced by the similarity in group average and individual subject Tₘₐₓ. In addition, there was no difference between the group mean profiles when compared on an individual time point basis, further demonstrating that posture had no effect on absorption. The systemic exposure of estradiol in TX-004HR 25 mcg was generally low and occurred regardless of whether the subjects were ambulatory or supine for 4 hours after dosing. An important advantage of the formulation is that a woman can be ambulatory almost immediately after the formulation is administered, as opposed to other known formulations that require a subject to remain in a supine position after administration. Generally, other known formulations direct administration before bed at night because of the requirement to be supine, which requirement is unnecessary in the pharmaceutical compositions disclosed herein because the pharmaceutical compositions disclosed herein adhere to the vaginal tissue, the capsule dissolves rapidly, and the formulation is released into the vagina and rapidly absorbed by the vaginal tissue. Because activity level does not adversely affect the systemic absorption of estradiol, the formulation of the invention gives the patient more flexibility with her dosing regimen.

### Example 13: Safety Results in Randomized Double-Blind Placebo-Controlled Multicenter Study

Safety endpoints in the study included vital signs, clinical laboratory tests (blood chemistry, hematology, hormone levels, urine analysis), ECG, physical and gynecological examination findings, pap smears, endometrial biopsies, and adverse events (AEs). AEs included undesirable medical conditions occurring at any time during all study phases including the washout period, whether or not a study treatment had been administered. An AE was considered treatment emergent if it occurred after study drug administration, or if it was pre-existing and worsened during 120 days post-dose follow up. Participants were given a diary with instructions to record product use, sexual activity, symptoms/complaints, and other medications. AEs, concomitant medications, and vital signs were recorded and assessed at each study visit from screening to week 12.

TX-004HR had a favorable safety profile and was well tolerated. No clinically significant differences in AEs were observed between treatment and placebo groups (Table 144). Headache was the most commonly reported TEAE, followed by vaginal discharge, nasopharyngitis, and vulvovaginal pruritus (Table 144). Headache was the only treatment-related TEAE that was numerically more frequent in women receiving TX-004HR than those receiving placebo (3.7% for 4-μg dose vs 3.1% for placebo). Vaginal discharge was reported by numerically fewer women in any of the TX-004HR groups than by women in the placebo group. Most TEAEs were mild to moderate in severity. Few participants (1.8%) discontinued the study due to AEs.

TABLE 144
Number (%) of treatment emergent adverse events (TEAE)
reported for ≥3% in any treatment arm of the safety population.

| | TX- | TX- | TX- | |
|---|---|---|---|---|
| | 004HR | 004HR | 004HR | |
| | 4 μg | 10 μg | 25 μg | Placebo |
| Preferred Term | (n = 191) | (n = 191) | (n = 190) | (n = 192) |
| Any subject with | 97 (50.8) | 94 (49.2) | 93 (48.9) | 111 (57.8) |
| reported TEAE | | | | |
| Headache | 12 (6.3) | 14 (7.3) | 6 (3.2) | 15 (7.8) |
| Vaginal discharge | 5 (2.6) | 6 (3.1) | 4 (2.1) | 13 (6.8) |
| Nasopharyngitis | 5 (2.6) | 6 (3.1) | 7 (3.7) | 10 (5.2) |
| Vulvovaginal | 4 (2.1) | 3 (1.6) | 7 (3.7) | 10 (5.2) |
| pruritus | | | | |
| Back pain | 9 (4.7) | 1 (0.5) | 4 (2.1) | 8 (4.2) |
| Urinary | 5 (2.6) | 5 (2.6) | 8 (4.2) | 4 (2.1) |
| tract infection | | | | |
| Upper respiratory | | | | |
| tract infection | 5 (2.6) | 6 (3.1) | 3 (1.6) | 5 (2.6) |
| Oropharyngeal | 1 (0.5) | 0 (0) | 6 (3.2) | 1 (0.5) |
| pain | | | | |

Nine serious TEAEs were reported in 8 subjects; however, none were considered related to treatment. Complete heart block, appendicitis, endophthalmitis, and chronic obstructive pulmonary disease were each reported by a different participant in the 25 μg group. Sinus node dysfunction and ankle fracture were both reported for one women, and arthralgia and malignant melanoma were each reported for one women in the 10 μg group. None of the women in the 4 μg group had reports of serious TEAEs. One woman in the placebo group was reported to have a cervical myelopathy. No deaths occurred during the study.

No diagnoses of endometrial hyperplasia or malignancy from endometrial biopsies were observed at week 12. Total cholesterol numerically decreased from baseline to week 12 by a mean of 0.024 mmol/L to 0.07 mmol/L in the treatment groups, and by 0.008 mmol/L in the placebo group. No clinically meaningful increases in triglycerides were observed in any active treatment groups compared with placebo. Sex hormone binding globulin (SHBG) concentrations (measured in a subset of 72 women) did not increase with treatment relative to placebo or baseline at week 12. No clinically significant changes in any laboratory parameters were found.

The phase 3 clinical trial demonstrated that TX-004HR at 4 μg, 10 μg, and 25 μg doses is safe and effective for treating vaginal changes and self-reported symptoms of VVA in postmenopausal women. Statistically significant and clinically meaningful improvements in all of the 4 pre-specified co-primary endpoints (increase in the percentage of vaginal superficial cells, decrease in the percentage of vaginal parabasal cells and vaginal pH, and decrease in severity of the MBS of dyspareunia) occurred as early as 2 weeks with all 3 doses of TX-004HR as compared with placebo, and were sustained throughout the 12-week trial. Additionally, improvements were found for the secondary endpoints of vaginal dryness and vulvar or vaginal irritation and itching. These improvements were achieved without increasing systemic estrogen concentrations (4 μg and 10 μg) or with negligible (25 μg) systemic estrogen exposure, as found in pharmacokinetic studies. TX-004HR was also well-tolerated with no clinically significant differences found between treatment and placebo groups in any AEs or treatment-related AEs, and no treatment-related serious AEs.

The results demonstrate early onset of action in the clinical signs of VVA with statistically significantly improved changes compared with placebo. The efficacy results here were somewhat numerically higher than data from a 12-week, randomized, controlled trial that compared a 10-μg vaginal estradiol tablet with placebo, which showed significant improvements in the percentages of superficial and parabasal cells, and in pH compared with placebo (see, Simon et al. Obstet Gynecol. 2008; 112:1053-1060). At 12 weeks, improvements were smaller with the 10-μg estradiol tablet (change of 13% in superficial cells, −37% in parabasal cells, and −1.3 in vaginal pH) than what was observed in this study with the 10-μg TX-004HR dose (change of 17% in superficial cells, −44% in parabasal cells, and −1.4 in vaginal pH). While improvements in some objective (cell and pH) endpoints were seen with the estradiol tablet within 2 weeks of treatment, the patient-reported improvements in a composite score of subjective symptoms were not observed until 8 weeks of therapy, which can be perceived as a disadvantage for many users. That clinical trial did not assess individual symptoms. A second randomized, controlled trial of 10-μg and 25-μg estradiol tablets similarly did not find significant improvements over placebo in the composite score of vaginal symptoms with either dose until 7 weeks of treatment (week 2, NS). Likewise, the SERM, ospemifene, was evaluated in a clinical trial for the treatment of dyspareunia, and statistically significant improvements were not observed until week 12. See, Bachmann et al. Obstet Gynecol. 2008; 111:67-76; Portman et al. Menopause. 2013; 20:623-630.

Importantly, the results reported here showed significant improvement in dyspareunia within 2 weeks with all 3 doses of TX-004HR, with reductions in severity scores from 1.5 to 1.7 points at week 12, which were comparable or superior to reductions of 1.2 to 1.6 points reported for other currently approved dyspareunia treatments. See, VAGIFEM® (estradiol vaginal tablets) Prescribing Information. Bagsvaerd, Denmark: Novo Nordisk Pharmaceuticals Inc.; 2012; PREMARIN® (conjugated estrogens tablets, USP) Prescribing Information. Philadelphia, PA: Wyeth Pharmaceuticals Inc.; 2010; OSPHENA® (ospemifene) tablets, for oral use. Prescribing Information. Shionogi, Inc. 2013.

Additionally, vaginal dryness improved from week 2 with 10 μg and 25 μg TX-004HR. None of the currently available products reported as early an onset of action for the symptom of vaginal dryness associated with VVA as did TX-004HR. Furthermore, TX-004HR 10 μg and 25 μg showed significant improvement in vaginal irritation and/or itching at week 12, while none of the currently available products on the market are reported to improve these symptoms. See, Portman et al. Maturitas. 2014; 78:91-98; Eriksen et al. Eur J Obstet Gynecol Reprod Biol. 1992; 44:137-144.

Based on a large survey of postmenopausal women in the United States, only a small proportion (7%) of women are thought to receive prescription vaginal estrogen therapy alone for their VVA, probably due to lack of information about available treatments, avoidance of discussion of the topic with health care practitioners, or dissatisfaction with currently available products (see, e.g., Kingsberg et al. J Sex Med. 2013; 10:1790-1799). Eliminating the need for an applicator or individually measuring doses is intended to give women a more positive user experience and thus potentially better compliance, resulting in overall better efficacy of treatment.

The results with TX-004HR in this study exemplify one of the advantages of local vaginal estrogen therapies: rapid symptom resolution without increasing systemic estrogen concentrations. The mean area under the concentration-time curve (AUC) and average concentration (C_{avg}) for estradiol were not significantly different from placebo with 4 μg and g TX-004HR. Although statistically higher AUC for estradiol was observed with the 25 μg dose, estradiol levels remained within the postmenopausal range with no evidence of accumulation by day 84. Although there was negligible systemic absorption, rapid efficacy was observed within 2 weeks of dosing with all doses of TX-004HR.

TX-004HR was well-tolerated. The 4 most commonly reported TEAEs, including vaginal discharge and vulvovaginal pruritus, were experienced by fewer women in any TX-004HR group than in the placebo group, and were mostly mild to moderate in severity. By comparison, in a 12-week study of the efficacy of ospemifene, vaginal discharge was reported more than 6-times more frequently in the ospemifene group than in the placebo group (see, Portman et al. Menopause. 2013; 20:623-630). Genital pruritus was also reported 4-times more frequently in women treated with Vagifem 10-μg tablets than with placebo in a 12-month randomized study (see, Vagifem® (estradiol vaginal tablets) Prescribing Information. Bagsvaerd, Denmark: Novo Nordisk Pharmaceuticals Inc.; 2012). Importantly, endometrial findings after TX-004HR were benign as no hyperplasia or malignancies were reported in biopsies at 12 weeks. Onset of effect was seen as early as 2 weeks and was maintained throughout the study. TX-004HR was well tolerated as reported here and systemic estrogen exposure was negligible to very low as demonstrated by the pharmacokinetic study.

### Example 14: Results of Female Sexual Function Index in Randomized, Double-Blind, Placebo-Controlled Multicenter Study

The trial was a randomized, double-blind, placebo controlled, multicenter, phase 3 study. Treatments were self-administered vaginally, once daily, for 2 weeks and then twice weekly, for 10 weeks. Female sexual dysfunction (FSD) was evaluated using the multidimensional Female Sexual Function Index (FSFI) at baseline and at week 12. The FSFI is a brief, validated, self-reporting questionnaire consisting of 19 questions designed to assess the areas of arousal, desire, orgasm, lubrication, and pain. The Index defines sexual dysfunction by a total FSFI score (the sum of the individual domain scores) of ≤26.55 out of a possible maximum score of 36.

Postmenopausal women (40-75 years; BMI ≤38 kg/m2) were included if they had ≤5% superficial cells on vaginal cytological smear; vaginal pH >5.0; self-identified most bothersome symptom (MBS) of moderate-to severe dyspareunia; and anticipated sexual activity (with vaginal penetration) during the trial period. Vulvar and vaginal atrophy (VVA) treatments, including vaginal lubricants and moisturizers, were discontinued within 7 days prior to screening. Use of oral estrogen-, progestin-, androgen-, or SERM-containing drug products were prohibited within 8 weeks of study start. Changes from baseline in total and individual domain FSFI scores for each dose were compared with placebo using ANCOVA with baseline as a covariate.

764 postmenopausal women were randomized to 4 μg (n=191), 10 μg (n=191), or 25 μg (n=190) vaginal estradiol softgel capsules or placebo (n=192). The majority of the women were white (87%) with a mean age of 59 years and a mean BMI of 26.7 kg/m² (Table 145). The FSFI questionnaire was completed by those who were not in the PK sub-study (n=692; 90.6%). The average baseline total FSFI score of 14.8 for all women indicated FSD in the subjects.

TABLE 145
Summary of subjects enrolled in study

| | Com- | Com- | Com- | |
|---|---|---|---|---|
| | position 4 | position 5 | position 6 | Com- |
| | 4 μg | 10 μg | 25 μg | position 7 |
| | (n = 186) | (n = 188) | (n = 186) | (n = 187) |
| Age, years | | | | |
| Mean ± SD | 59.8 ± 6.0 | 58.6 ± 6.3 | 58.8 ± 6.2 | 59.4 ± 6.0 |
| Race, n (%) | | | | |
| White | 162 (87.1) | 165 (87.8) | 161 (86.6) | 160 (85.6) |
| Black or | 20 (10.8) | 21 (11.2) | 24 (12.9) | 21 (11.2) |
| African | | | | |
| American | | | | |
| Asian | 3 (1.6) | 2 (1.1) | 1 (0.5) | 1 (0.5) |
| BMI, kg/m² | | | | |
| Mean ± SD | 26.6 ± 4.9 | 26.8 ± 4.7 | 26.9 ± 4.8 | 26.6 ± 4.6 |
| Baseline total | | | | |
| FSFI Score | | | | |
| Mean ± SD | 14.8 ± 6.13 | 15.8 ± 6.24 | 14.2 ± 6.21 | 14.4 ± 6.61 |
| Baseline FSFI | | | | |
| Pain Score | | | | |
| Mean ± SD | 1.6 ± 1.11 | 1.8 ± 1.22 | 1.7 ± 1.17 | 1.7 ± 1.20 |

The Female Sexual Function Index (FSFI) total summary score is a numerically continuous measure that was descriptively summarized at Visits 2 and 6 and the change in the total summary score (Visit 6 minus Visit 2) was also descriptively summarized. The domain sub-scores and the changes in the domain sub-scores were also descriptively summarized. Summaries were by treatment arm, and all active treatment arms combined.

In addition, the change in mean from baseline of each active treatment group from the placebo group for each numerically continuous endpoint was evaluated. The least square (LS) mean changes and the 95% CI for the difference in LS Mean changes between treated and placebo are provided. The FSFI Questionnaire consists of 19 questions divided among 6 domains, and has a minimum total score of 2.0 and a maximum score of 36.0 points. The FSFI questionnaire was administered to the randomized population except for those subjects in the PK sub-study. At Baseline, the overall mean Total Score was 14.8 (14.8 for the 4 μg group; 15.8 for the 10 μg group; 14.2 for the 25 μg group; and 14.4 for the placebo group). The LS mean change in the FSFI Total Score and domain scores from Baseline to Week 12 are summarized in Table 146.

Change from Baseline to Week 12 in FSFI total score and domains compared to placebo was assessed.

After 12 weeks, total FSFI scores numerically improved from baseline in all groups, including placebo. Total FSFI score significantly increased with the 10 μg group (P<0.05) and the 25 μg group (P=0.0019) versus placebo (FIG. 24).

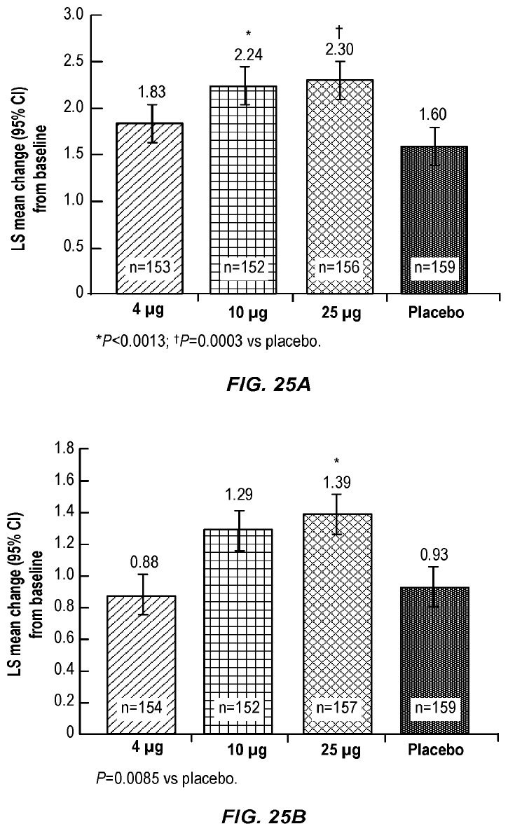

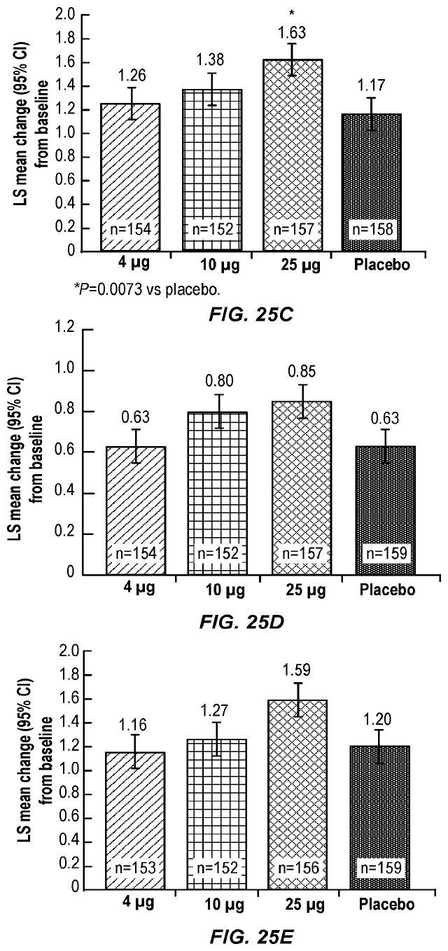

FSFI lubrication and pain domain scores improved numerically in all groups including placebo from baseline to 12 weeks; improvements for the 10 μg group and the 25 μg group were statistically significantly greater than with placebo (FIG. 25A). The 25 μg composition significantly improved FSFI arousal (P=0.0085) and satisfaction (P=0.0073) domain scores at 12 weeks (FIG. 25B, FIG. 25C). All three doses were comparable to placebo in their effect on the FSFI domains of desire and orgasm (FIG. 25D, FIG. 25E). The 4 μg composition and placebo provided similar levels of improvement. The compositions improved FSFI in a dose-dependent manner, with the 25 μg dose having the greatest improvement. All three doses were efficacious, and the numeric improvement in subjective symptoms was highest for subjects in the 10 and 25 μg groups. The observed placebo response could be attributed to the coconut oil (Miglyol) in the formulation for the placebo and the estradiol compositions, which may also contribute to the observed benefits

TABLE 146
Female Sexual Function Index Total and Domain Scores:

| | | 4 μg | | 10 μg | | 25 μg | | Placebo | |
|---|---|---|---|---|---|---|---|---|---|
| Category | Score | Mean | SD | Mean | SD | Mean | SD | Mean | SD |
| Total | Baseline | 14.8 | 6.13 | 15.8 | 6.24 | 14.2 | 6.21 | 14.4 | 6.61 |
| | Week 12 | 22.6 | 8.4 | 24.8 | 7.59 | 24.8 | 7.59 | 22 | 8.54 |
| | Change | 7.98 | 7.551 | 8.85 | 7.361 | 10.49 | 8.176 | 7.74 | 8.41 |
| | LS Mean | 7.909 | 0.9075 | 9.431 | 0.0492 | 10.283 | 0.0019 | 7.458 | — |
| Arousal | Baseline | 2.8 | 1.44 | 2.9 | 1.43 | 2.7 | 1.5 | 2.7 | 1.41 |
| | Week 12 | 3.6 | 1.61 | 4.1 | 1.47 | 4.1 | 1.39 | 3.6 | 1.52 |
| | Change | 0.88 | 1.615 | 1.16 | 1.632 | 1.43 | 1.646 | 1.02 | 1.607 |
| | LS Mean | 0.876 | 0.9777 | 1.288 | 0.0581 | 1.393 | 0.008 | 0.927 | — |
| Desire | Baseline | 2.6 | 1.01 | 2.7 | 1.13 | 2.6 | 1.09 | 2.7 | 1.07 |
| | Week 12 | 3.3 | 1.11 | 3.5 | 1.13 | 3.5 | 1.06 | 3.3 | 1.21 |
| | Change | 0.64 | 1.065 | 0.78 | 1.113 | 0.87 | 1.105 | 0.62 | 1.102 |
| | LS Mean | 0.626 | 1 | 0.801 | 0.2753 | 0.849 | 0.1139 | 0.628 | — |
| Lubrication | Baseline | 2.1 | 1.25 | 2.3 | 1.25 | 2 | 1.19 | 2 | 1.29 |
| | Week 12 | 3.9 | 1.84 | 4.4 | 1.56 | 4.3 | 1.65 | 3.6 | 1.77 |
| | Change | 1.84 | 1.782 | 2.12 | 1.612 | 2.36 | 1.744 | 1.64 | 1.871 |
| | LS Mean | 1.835 | 0.4023 | 2.243 | 0.0012 | 2.3 | 0.0003 | 1.591 | — |
| Orgasm | Baseline | 2.7 | 1.74 | 2.9 | 1.74 | 2.4 | 1.68 | 2.4 | 1.73 |
| | Week 12 | 3.8 | 1.89 | 4.1 | 1.75 | 4.1 | 1.66 | 3.7 | 1.97 |
| | Change | 1.12 | 1.93 | 1.09 | 1.821 | 1.68 | 1.857 | 1.31 | 1.86 |
| | LS Mean | 1.162 | 0.9978 | 1.273 | 0.9424 | 1.59 | 0.0763 | 1.189 | — |
| Satisfaction | Baseline | 2.9 | 1.37 | 3.2 | 1.43 | 2.9 | 1.37 | 2.9 | 1.49 |
| | Week 12 | 4.2 | 1.54 | 4.4 | 1.37 | 4.6 | 1.35 | 4.1 | 1.55 |
| | Change | 1.31 | 1.512 | 1.24 | 1.534 | 1.64 | 1.613 | 1.23 | 1.661 |
| | LS Mean | 1.256 | 0.8798 | 1.382 | 0.3484 | 1.628 | 0.0063 | 1.165 | — |

While the pharmaceutical compositions and methods have been described in terms of what are presently considered to be practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar embodiments. This disclosure includes any and all embodiments of the following claims.

What is claimed is:

1. A method of treating vulvovaginal atrophy (VVA), comprising vaginally administering to a subject in need thereof estradiol at a dosage of 4 mcg and increasing the dosage of estradiol if the subject does not exhibit an improvement in a clinical response for VVA.

2. The method of claim 1, wherein the estradiol is administered once daily for two weeks, then twice weekly thereafter.

3. The method of claim 1, wherein the estradiol is solubilized.

4. The method of claim 1, wherein a dosage form comprising the estradiol is manually inserted about two inches into the vagina.

5. The method of claim 4, wherein the estradiol is formulated in a soft gelatin capsule.

6. The method of claim 1, comprising increasing the dosage of estradiol to 10 mcg if the subject does not exhibit an improvement in the clinical response.

7. The method of claim 1, comprising increasing the dosage of estradiol to 25 mcg if the subject does not exhibit an improvement in the clinical response.

8. The method of claim 1, comprising maintaining the dosage of estradiol if an improvement in a clinical response is achieved.

9. The method of claim 1, comprising decreasing the dosage of estradiol if an improvement in a clinical response is achieved.

10. A method of treating vulvovaginal atrophy (VVA), comprising vaginally administering to a subject in need thereof estradiol once daily for two weeks, then twice weekly thereafter, wherein the estradiol is administered at a dosage of 4 mcg.

11. The method of claim 10, wherein the estradiol is solubilized.

12. The method of claim 10, wherein a dosage form comprising the estradiol is manually inserted about two inches into the vagina.

13. The method of claim 12, wherein the estradiol is formulated in a soft gelatin capsule.

14. A method of treating moderate to severe dyspareunia, comprising vaginally administering to a subject in need thereof estradiol at a dosage of 4 mcg and increasing the dosage of estradiol if the subject does not exhibit an improvement in the moderate to severe dyspareunia.

15. The method of claim 14, wherein the estradiol is administered once daily for two weeks, then twice weekly thereafter.

16. The method of claim 14, wherein the estradiol is solubilized.

17. The method of claim 14, wherein the estradiol is formulated in a soft gelatin capsule.

18. The method of claim 14, comprising increasing the dosage of estradiol to 10 mcg if the subject does not exhibit an improvement in the moderate to severe dyspareunia.

19. The method of claim 14, comprising increasing the dosage of estradiol to 25 mcg if the subject does not exhibit an improvement in the moderate to severe dyspareunia.
